(19) United States
(12) Patent Application Publication
(10) Pub. No.: US 2008/0064052 A1
(43) Pub. Date: Mar. 13, 2008

(54) Crystal of a Receptor-Ligand Complex and methods of use

(75) Inventors: Peter Kuhn, Solana Beach, CA (US); Anand Kolaktar, San Diego, CA (US); Alexei Brooun, San Diego, CA (US); Jill Chrencik, San Diego, CA (US); Michelle Kraus, Temecula, CA (US)

Correspondence Address: BIOTACTICA, LLC, 10733 Sunset Office Drive, Suite 261, ST. LOUIS, MO, 63127, US

(73) Assignee: The Scripps Research Institute, La Jolla, CA (US)

(21) Appl. No.: 11/622,785

(22) Filed: Jan. 12, 2007

Related U.S. Application Data
(60) Provisional application No. 60/759,167, filed on Jan. 12, 2006.

(51) Int. Cl. G01N 33/00; C07K 1/00; G06G 7/48

(52) U.S. Cl. 435/7.92; 530/350; 703/11

(57) ABSTRACT

The invention relates to the three-dimensional structure of a crystal of an EphB4 receptor complexed with a ligand. The three-dimensional structure of a Receptor-Ligand Complex is disclosed. The receptor-ligand crystal structure, wherein the ligand is an inhibitor molecule, is useful for providing structural information that may be integrated into drug screening and drug design processes. Thus, the invention also relates to methods for utilizing the crystal structure of the Receptor-Ligand Complex for identifying, designing, selecting, or testing inhibitors of the EphB4 receptor protein, such inhibitors being useful as therapeutics for the treatment or modulation of i) diseases; ii) disease symptoms; or iii) the effect of other physiological events mediated by the receptor.

### CROSS-REFERENCE TO RELATED APPLICATIONS

This application claims priority to U.S. Provisional Application 60/759,167 filed Jan. 12, 2006 which is hereby incorporated by reference in its entirety.

### STATEMENT REGARDING FEDERALLY SPONSORED RESEARCH OR DEVELOPMENT

Not Applicable.

### INCORPORATION-BY-REFERENCE OF SEQUENCE LISTING

The Sequence Listing, which is a part of the present disclosure, is a written sequence listing comprising nucleotide and amino acid sequences of the present invention. The subject matter of the Sequence Listing is incorporated herein by reference in its entirety.

### FIELD

The present invention relates to a three-dimensional structure of a receptor tyrosine kinase from the erythropoietin-producing hepatocellular carcinoma family of receptor tyrosine kinases (“Eph”), particularly an EphB complexed with an ephrin ligand (“Receptor-Ligand Complex”), for example an EphB4 or similar polypeptide complexed with an ephrin-B2 or analog, three-dimensional coordinates of a Receptor-Ligand Complex, models thereof, and uses of such structures and models.

### INTRODUCTION

The Eph receptor tyrosine kinases and their ligands, the ephrins, regulate numerous biological processes in developing and adult tissues and have been implicated in cancer progression and in pathological forms of angiogenesis. For example, the Eph receptors and their ligands, the ephrins, play critical roles in angiogenesis during embryonic development as well as in adult tissues (Brantley-Sieders and Chen, 2004; Cheng et al., 2002; Gale and Yancopoulos, 1999; Kullander and Klein, 2002). The Eph family of receptor tyrosine kinases also regulates many other biological processes, including tissue patterning, axonal guidance, and as more recently discovered, tumorigenesis (Carmeliet and Collen, 1999; Ferrara, 1999; Pasquale, 2005; Wilkinson, 2000). Both the Eph receptor and the ephrin are membrane bound, and therefore require cell-cell contact to signal a cellular response. The interaction between Eph receptors and ephrins on adjacent cell surfaces results in multimerization and clustering of the Eph-ephrin complexes, leading to forward signaling in the Eph-expressing cell and reverse signaling in the ephrin-expressing cell. EphB4 belongs to the Eph (erythropoietin-producing hepatocellular carcinoma) family of receptor tyrosine kinases, which is divided into two subclasses, A and B, based on binding preferences and sequence conservation (Gale et al., 1996). In general, EphA receptors (EphA1-EphA10) bind to glycosyl phosphatidyl in ositol-(GPI) anchored ephrin-A ligands (ephrin-A1-ephrin-A6), while EphB receptors (EphB1-EphB6) interact with transmembrane ephrin-B ligands (ephrin-B1-ephrin-B3) (Eph Nomenclature Committee, 1997). While interactions between the Eph receptors and ephrin ligands of the same subclass are quite promiscuous, interactions between subclasses are rare. A few cross-subclass exceptions include the EphA4-ephrin-B2/B3 interactions (Takemoto et al., 2002), and the EphB2-ephrinA5 interaction, which has been characterized structurally (Himanen et al., 2004). EphB4 is unique within the Eph family in that it selectively binds ephrin-B2, while demonstrating only weak binding for both ephrin-B1 and ephrin-B3.

Eph receptors have a modular structure, consisting of an N-terminal ephrin binding domain adjacent to a cysteine-rich domain and two fibronectin type III repeats in the extracellular region. The intracellular region consists of a juxtamembrane domain, a conserved tyrosine kinase domain, a C-terminal sterile α-domain (SAM), and a PDZ binding motif. The N-terminal 180 amino acid globular domain is sufficient for high-affinity ligand binding (Himanen et al., 2001).

Several 12-amino-acid peptides that selectively bind to individual Eph receptors were recently identified by phage display (Koolpe et al., 2005; Koolpe et al., 2002; Murai et al., 2003). A number of EphB4-binding peptides could be aligned with each other and the 15 amino acid segment corresponding to the ephrin-B2 G-H loop (Koolpe et al., 2005). The TNYL EphB4-binding peptide was modified based on this alignment to include a carboxy-terminal RAW sequence. The resulting TNYL-RAW (TNYLFSPNGPIARAW; SEQ ID NO: 1) peptide is a potent antagonist of ephrin-B2 binding to EphB4, with an IC₅₀ value of ˜15 nM for the murine receptor, which is comparable to the IC₅₀ of ˜10 nM measured for ephrin-B2 (Table 1A). Interestingly, the TNYL peptide (which lacks the carboxy-terminal RAW sequence) is 10,000-fold less potent than TNYL-RAW (IC₅₀ of ˜150 μM).

Despite attempts to model the structural changes of EphB4 upon ligand binding, a detailed view of conformational arrangements of an EphB4 receptor in complex with a highly-selective ligand has remained elusive. Thus, the development of useful reagents for treatment or diagnosis of disease was hindered by lack of structural information of such a Receptor-Ligand Complex. Therefore, there is a need in the art to elucidate the three-dimensional structure and models of Receptor-Ligand Complexes, and to use such structures and models in therapeutic strategies, such as drug design.

### SUMMARY

The present teachings include a method for designing a drug which interferes with an activity of an EphB4 receptor, the method comprising providing on a digital computer a three-dimensional structure of a receptor-ligand complex comprising the EphB4 receptor and at least one ligand of the EphB4 receptor, and using software comprised by the digital computer to design a chemical compound which is predicted to bind to the EphB4 receptor. The method can further comprise synthesizing the chemical compound, and evaluating the chemical compound for an ability to interfere with an activity of the EphB4 receptor.

In accordance with a further aspect, the chemical compound of the method is designed by computational interaction with reference to a three-dimensional site of the structure of the receptor-ligand complex. The three-dimensional site can include EphB4 D-E and J-K loops. The three-dimensional site can also include Leu-48, Cys-61, Leu-95, Ser-99Leu-100, Pro-101, Thr-147, Lys-149, Ala-155, and Cys-184 of human EphB4 (SEQ ID NO:26). In another aspect, the EphB4 receptor is a human EphB4 receptor.

The present teachings also include a method for determining a three-dimensional structure of a target EphB receptor-ligand complex structure comprising providing an amino acid sequence of a target EphB structure, wherein the three-dimensional structure of the target EphB structure is not known, predicting a pattern of folding of the amino acid sequence in a three-dimensional conformation using a fold recognition algorithm, and comparing the pattern of folding of the target structure amino acid sequence with the three-dimensional structure of a known EphB4 receptor-ligand complex. In certain aspects, the EphB4 receptor comprises a truncated EphB4 receptor, such as EphB4 (17-196) as set forth in SEQ ID NO: 2, and other homologs and analogs such as EphB4 (17-198) as set forth in SEQ ID NO: 3. In certain aspects, the EphB4 receptor consists essentially of an amino acid sequence as set forth in SEQ ID NO: 2 and other homologs and analogs such asEphB4 (17-198) as set forth in SEQ ID NO: 3. In certain aspects, the known receptor-ligand complex comprises a three-dimensional structure described by atomic coordinates that substantially conform to atomic coordinates set forth in Table 1. In additional aspects, the EphB4 receptor is a human EphB4 receptor.

In accordance with yet another aspect, a method is provided for generating a model of a three-dimensional structure of an EphB-ligand complex, the method comprising providing an amino acid sequence of a reference EphB4 polypeptide and an amino acid sequence of a target EphB comprised by the EphB-ligand complex, identifying structurally conserved regions shared between the reference EphB4 amino acid sequence and the target EphB amino acid sequence, and assigning atomic coordinates from the conserved regions to the target EphB-ligand complex. In certain aspects, the EphB4 polypeptide comprises a truncated EphB4 receptor, such as EphB4 (17-196) as set forth in SEQ ID NO:2, and other homologs and analogs such as EphB4 (17-198) as set forth in SEQ ID NO: 3. In certain aspects, the EphB4 polypeptide consists essentially of an amino acid sequence asset forth in SEQ ID NO: 2, and other homologs and analogs such as EphB4 (17-198) as set forth in SEQ ID NO: 3. In certain aspects, the target EphB-ligand complex comprises a three-dimensional structure described by atomic coordinates that substantially conform to atomic coordinates set forth in Table 1. In certain aspects, the reference EphB4-ligand complex comprises a three-dimensional structure described by atomic coordinates that substantially conform to atomic coordinates set forth in Table 1. In additional aspects, the EphB4 polypeptide is a human EphB4 polypeptide.

In accordance with another aspect, a method is provided for generating a model of a three-dimensional structure of an EphB receptor-ligand complex, the method comprising providing an amino acid sequence of a known EphB4 receptor in complex with at least one known ligand of the EphB4 receptor, providing an amino acid sequence of a target EphB receptor in complex with at least one target ligand of the EphB receptor, identifying structurally conserved regions shared between the known receptor-ligand complex amino acid sequence and the target receptor-ligand complex amino acid sequence, and assigning atomic coordinates of the conserved regions to the target receptor-ligand complex. In certain aspects, the EphB4 receptor comprises a truncated EphB4 receptor, such as EphB4(17-196) as set forth in SEQ ID NO: 2, and other homologs and analogs such as EphB4 (17-198) as set forth in SEQ ID NO: 3. In certain aspects, the EphB4 receptor consists essentially of an amino acid sequence as set forth in SEQ ID NO: 2 and other homologs and analogs such as EphB4 (17-198) as set forth in SEQ ID NO: 3. In certain aspects, the EphB4 receptor is a human EphB4 receptor. In additional aspects, the known receptor-ligand complex comprises a three-dimensional structure described by atomic coordinates that substantially conform to Table 1.

According to another aspect, a crystal is provided consisting essentially of an EphB4 ligand binding domain and a ligand. In certain aspects, the EphB4 ligand binding domain is a truncated EphB4 polypeptide having the sequence of SEQ ID NO: 2 and other homologs and analogs such as EphB4 (17-198) as set forth in SEQ ID NO: 3. In certain aspects, the EphB4 ligand binding domain consists essentially of EphB4 D-E and J-K loops. In certain aspects, the EphB4 ligand binding domain consists essentially of Leu-48, Cys-61, Leu-95, Ser-99 Leu-100, Pro-101, Thr-147, Lys-149, Ala-155, and Cys-184 of human EphB4(SEQ ID NO: 27). In certain aspects, the EphB4 ligand binding domain is a human EphB4 ligand binding domain. In additional aspects, the ligand is ephrin-B2. In certain aspects, the ligand comprises Phe-120, Pro-122, Leu-124, Trp-125, and Leu-127 of human ephrin-B2 (SEQ ID NO: 29). In certain aspects, the ligand comprises sequence motif NxWxL, wherein x is any amino acid. In certain aspects, the ligand is TNYL-RAW, a polypeptide having SEQ ID NO: 1. In other aspects, the ligand is a polypeptide selected from the group consisting of polypeptides having SEQ ID NO: 4 through SEQ ID NO: 26. In certain aspects, the ligand is a polypeptide having at least 50% sequence identity to a polypeptide selected from the group consisting of polypeptides having SEQ ID NO: 1 and SEQ ID NO: 4 through SEQ ID NO: 26. In certain aspects, the ligand is a polypeptide having at least 75% sequence identity to a polypeptide selected from the group consisting of polypeptides having SEQ ID NO: land SEQ ID NO: 4 through SEQ ID NO: 26. In certain other aspects, the ligand is a polypeptide having at least 90% sequence identity to a polypeptide selected from the group consisting of polypeptides having SEQ ID NO: 1 and SEQ ID NO: 4 through SEQ ID NO: 26. In additional aspects, the crystal comprises space group P41212 so as to form a unit cell of dimensions a=60.97 Å, b=60.97 Å, and c=151.7 Å.

In yet another aspect, a crystal is provided comprising a polypeptide having SEQ ID NO: 2 or 3 complexed with a ligand, wherein the crystal is sufficiently pure to determine atomic coordinates of the complex by X-ray diffraction to a resolution of about 1.65 Å. In certain aspects, the ligand comprises Phe-120, Pro-122, Leu-124, Trp-125, and Leu-127 of ephrin-B2. In certain aspects, the ligand comprises sequence motif NxWxL, wherein x is any amino acid. In certain aspects, the ligand is a polypeptide having SEQ ID NO: 1. In certain aspects, the ligand is a polypeptide selected from the group consisting of polypeptides having SEQ ID NO: 4 through SEQ ID NO: 26. In certain aspects, the ligand is a polypeptide having at least 50% sequence identity to a polypeptide selected from the group consisting of polypeptides having SEQ ID NO: 1 and SEQ ID NO: 4 through SEQ ID NO: 26. In certain aspects, the ligand is a polypeptide having at least 75% sequence identity to a polypeptide selected from the group consisting of polypeptides having SEQ ID NO: 1 and SEQ ID NO: 4 through SEQ ID NO: 26. In certain aspects, the ligand is a polypeptide having at least 90% sequence identity to a polypeptide selected from the group consisting of polypeptides having SEQ ID NO: 1 and SEQ ID NO: 4 through SEQ ID NO: 26.

In yet another aspect, a polypeptide is provided having SEQ ID NO: 2 or 3 in complex with a ligand. In certain aspects, the ligand is an ephrin. In certain aspects, the ephrin is ephrin-B2. In certain aspects, the ligand comprises Phe-120, Pro-122, Leu-124, Trp-125, and Leu-127 of ephrin-B2. In certain aspects, the ligand comprises sequence motif NxWxL, wherein x is any amino acid. In certain aspects, the ligand is a polypeptide having SEQ ID NO: 1. In certain aspects, the ligand is a polypeptide selected from the group consisting of polypeptides having SEQ ID NO: 4 through SEQ ID NO: 26. In certain aspects, the ligand is a polypeptide having at least 50% sequence identity to a polypeptide selected from the group consisting of polypeptides having SEQ ID NO: 1 and SEQ ID NO: 4 through SEQ ID NO: 26. In certain aspects, the ligand is a polypeptide having at least 75%sequence identity to a polypeptide selected from the group consisting of polypeptides having SEQ ID NO: 1 and SEQ ID NO: 4 through SEQ ID NO: 26. In certain aspects, the ligand is a polypeptide having at least 90% sequence identity to a polypeptide selected from the group consisting of polypeptides having SEQ ID NO: 1 and SEQ ID NO: 4 through SEQ ID NO: 26.

In other aspects, a therapeutic compound is provided that inhibits an activity of an EphB4 receptor, wherein the compound is selected by performing a structure based drug design using a three-dimensional structure determined for a crystal comprising an EphB4 receptor and a ligand, contacting a sample comprising the EphB4 receptor with the compound, and detecting inhibition of at least one activity of the EphB4 receptor. In certain aspects, the EphB4 is a polypeptide having SEQ ID NO: 2 or 3. In certain aspects, the EphB4 receptor is a human EphB4 receptor. In certain aspects, the ligand is a polypeptide having SEQ ID NO: 1. In certain aspects, the ligand is a polypeptide selected from the group consisting of polypeptides having SEQ ID NO: 4 through SEQ ID NO: 26. In certain aspects, the ligand is a polypeptide having at least 50% sequence identity to a polypeptide selected from the group consisting of polypeptides having SEQ ID NO: 1 and SEQ ID NO: 4 through SEQ ID NO: 26. In certain aspects, the ligand is a polypeptide having at least 75% sequence identity to a polypeptide selected from the group consisting of polypeptides having SEQ ID NO: 1 and SEQ ID NO: 4 through SEQ ID NO: 26. In certain aspects, the ligand is a polypeptide having at least 90% sequence identity to a polypeptide selected from the group consisting of polypeptides having SEQ ID NO: 1 and SEQ ID NO: 4 through SEQ ID NO: 26.

In yet another aspect, a three-dimensional computer image of the three-dimensional structure of an EphB4-ligand complex is provided wherein the structure substantially conforms to the three-dimensional coordinates listed in Table 1.

In yet another aspect, a computer-readable medium encoded with a set of three-dimensional coordinates set forth in Table 1 is provided wherein, using a graphical display software program, the three-dimensional coordinates of Table 1 create an electronic file that can be visualized on a computer capable of representing said electronic file as a three-dimensional image.

In yet another aspect, a computer-readable medium encoded with a set of three-dimensional coordinates of a three-dimensional structure which substantially conforms to the three-dimensional coordinates represented in Table 1 is provided wherein, using a graphical display software program, the set of three-dimensional coordinates create an electronic file that can be visualized on a computer capable of representing said electronic file as a three-dimensional image.

In yet another aspect, a method is provided for assaying EphB4 receptor binding to a compound, the method comprising providing an EphB4 receptor bound with a polypeptide having SEQ ID NO: 1, contacting the ligand-bound EphB4 receptor with a compound, and detecting the release of the polypeptide having SEQ ID NO: 1 from the EphB4 receptor, wherein the release of the polypeptide having SEQ ID NO: 1 is indicative of the compound binding to the EphB4 receptor. In certain aspects, the EphB4 receptor is a polypeptide having SEQ ID NO: 2 or 3. In certain aspects, the EphB4 receptor consists essentially of EphB4 D-E and J-K loops. In certain aspects, the EphB4 receptor consists essentially of Leu-48, Cys-61, Leu-95, Ser-99 Leu-100, Pro-101, Thr-147, Lys-149, Ala-155, and Cys-184 of SEQ ID NO: 27. In certain aspects, the EphB4 receptor is a human EphB4 receptor.

In another aspect, a method is provided for crystallizing an EphB4 receptor, the method comprising providing an EphB4 receptor in contact with a first polypeptide having SEQ ID NO: 1, and contacting the EphB4 receptor in contact with the first polypeptide with a second polypeptide having at least 50% sequence identity to SEQ ID NO: 1, but not identical to SEQ ID NO: 1, wherein the EphB4 receptor in contact with the first and second polypeptides forms an EphB4 receptor crystal. In certain aspects, the second polypeptide comprises at least 75% sequence identity to SEQ ID NO: 1. In certain aspects, the second polypeptide comprises at least 90% sequence identity to SEQ ID NO: 1.

In yet another aspect, a method is provided for crystallizing an EphB4 receptor, the method comprising providing an EphB4 receptor in contact with a polypeptide having SEQ ID NO: 1, and contacting the EphB4 receptor in contact with the polypeptide with a compound provided above, wherein the EphB4 receptor in contact with the polypeptide and the compound forms an EphB4 receptor crystal.

In another aspect, a composition is provided comprising EphB4 receptor, a ligand, and a compound provided above. In certain aspects, the EphB4 receptor is a polypeptide having SEQ ID NO: 2 or 3. In certain aspects, the EphB4 receptor consists essentially of EphB4 D-E and J-K loops. In certain aspects, the EphB4 receptor consists essentially of Leu-48, Cys-61, Leu-95, Ser-99 Leu-100, Pro-101, Thr-147, Lys-149, Ala-155, and Cys-184 of SEQ ID NO: 27. In certain aspects, the EphB4 receptor is a human EphB4 receptor. In certain aspects, the ligand is a polypeptide having SEQ ID NO: 1. In certain aspects, the ligand is a polypeptide selected from the group consisting of polypeptide shaving SEQ ID NO: 4 through SEQ ID NO: 26. In certain aspects, the ligand is a polypeptide having at least 50% sequence identity to a polypeptide selected from the group consisting of polypeptides having SEQ ID NO: 1 and SEQ ID NO: 4 through SEQ ID NO: 26. In certain aspects, the ligand is a polypeptide having at least 75% sequence identity to a polypeptide selected from the group consisting of polypeptides having SEQ ID NO: 1 and SEQ ID NO: 4 through SEQ ID NO: 26. In additional aspects, the ligand is a polypeptide having at least 90% sequence identity to a polypeptide selected from the group consisting of polypeptides having SEQ ID NO: 1 and SEQ ID NO: 4 through SEQ ID NO: 26.

These and other features, aspects and advantages of the present teachings will become better understood with reference to the following description, examples and appended claims.

### DRAWINGS

Those of skill in the art will understand that the drawings, described below, are for illustrative purposes only. The drawings are not intended to limit the scope of the present teachings in any way.

FIG. 1. The ephrin binding domain of the EphB4 receptor in complex with an antagonistic peptide, TNYL-RAW. Upper left panel, composite image of complex; upper right panel, β-sheets and peptide; lower left panel, loops; lower right panel, α-helices. The ephrin binding domain consists of a jellyroll folding topology with 13 anti-parallel B-sheets connected by loops of varying lengths. Peptide binding orders the D-E and J-K loops, which cannot be visualized in the apo structure of the related EphB2 receptor.

FIG. 2. Superposition of the EphB4 receptor on the EphB2 receptor from the EphB2-ephrin-B2 structure (Himanen et al., 2001; Protein Data Base Accession No. 1KGY, incorporated herein by reference in its entirety). The structures are superimposed with an overall r.m.s.d. of 1.08 Å between equivalent Cα positions. The J-K loop is displaced by as much as 20 Å in EphB4 compared to EphB2.

FIG. 3. Model of the EphB4-ephrin-B2 complex. The EphB4 receptor is predicted to form interactions similar to those previously described in the EphB2-ephrin-B2 complex. Although several interactions are likely absent in the EphB4-ephrin-B2 complex compared to the EphB2-ephrin-B2 complex, the tetramer is likely to form at high EphB4 and ephrin-B2 concentrations.

FIG. 4. Stereoview of sigma-A weighted 2|F_{obs}|-|F_{calc}| electron density at 1.65 Å resolution, contoured at 1 a for the antagonistic TNYL-RAW peptide (SEQ ID NO: 1). The peptide was placed into the density after an initial round of structure refinement. The N-terminal threonine lacks clear electron density and is therefore absent from the structure.

FIG. 5. Close-up of the binding interface of a model of the EphB4-ephrinB2 (SEQ ID NO: 27, and SEQ ID NO: 29 respectively) complex. Position of the peptide is distinct from the ephrinB2 G-H loop. Both peptide and ephrin G-H loop reside within the hydrophobic binding cleft of the EphB4 receptor.

FIG. 6. EphB4-TNYL interactions. Non-covalent interactions are indicated by dashed lines.

FIG. 7. Superposition of the TNYL-RAW peptide (SEQ ID NO: 1) on the EphB4 (surface)-ephrin-B2 model. The ligand G-H loop extends into the hydrophobic binding cleft of the EphB4 receptor such that the TNYL-RAW peptide (SEQ ID NO: 1) and the ephrin-B2 G-H loop compete for the same binding site. The peptide binds distinctly within the binding cleft, inhibiting ephrin-B2 (SEQ ID NO: 29) binding at both high affinity dimerization interfaces.

### DETAILED DESCRIPTION

The present invention relates to the discovery of the three-dimensional structure of a Receptor-Ligand Complex, models of such three-dimensional structures, a method of structure-based drug design using such structures, the compounds identified by such methods and the use of such compounds in therapeutic compositions. In particular, the present invention involves the crystal structure of the EphB4 receptor in complex with a highly specific antagonistic peptide at a resolution of 1.65 Å. The peptide is situated in a hydrophobic cleft of EphB4 corresponding to the cleft in EphB2 occupied by the ephrin-B2G-H loop. The crystal reveals structural features of EphB4 that, when in complex a ligand, provides a basis for antagonist design and modeling.

In particular, the structural and thermodynamic characterization of the EphB4 receptor in complex with a polypeptide having SEQ ID NO: 1 is described. The polypeptide is situated in the same hydrophobic cleft occupied by the ephrinB2 G-H loop, assuming a position distinct from this loop and preventing ligand binding interactions at two high-affinity dimerization interfaces. Although the peptide binds independently from the ephrin ligand, the interactions within the binding cleft are remarkably similar to previous complex structures, providing a stable network of interactions for binding. Further, structural analysis reveals the molecular determinants for the directed specificity of this antagonist for the EphB4 receptor, allowing the first insights into modulating pathways resulting in tumorigenesis and angiogenesis that rely on EphB4-ephrinB2 signaling.

One aspect of the present invention includes a model of a Receptor-Ligand Complex in which the model represents a three-dimensional structure of a Receptor-Ligand Complex. Another aspect of the present invention includes the three-dimensional structure of a Receptor-Ligand Complex. A three-dimensional structure of a Receptor-Ligand Complex substantially conforms with the atomic coordinates represented in Table 1. According to the present invention, the use of the term “substantially conforms” refers to at least a portion of a three-dimensional structure of a Receptor-Ligand Complex which is sufficiently spatially similar to at least a portion of a specified three-dimensional configuration of a particular set of atomic coordinates (e.g., those represented by Table 1) to allow the three-dimensional structure of a Receptor-Ligand Complex to be modeled or calculated using the particular set of atomic coordinates as a basis for determining the atomic coordinates defining the three-dimensional configuration of a Receptor-Ligand Complex.

More particularly, a structure that substantially conforms to a given set of atomic coordinates is a structure wherein at least about 50% of such structure has an average root-mean-square deviation (RMSD) of less than about 1.8 Å for the backbone atoms in secondary structure elements in each domain, and in various aspects, less than about 1.25 Å for the backbone atoms in secondary structure elements in each domain, and, in various aspects less than about 1.0 Å, in other aspects less than about 0.75 Å, less than about 0.5 Å, and, less than about 0.25 Å for the backbone atoms in secondary structure elements in each domain. In one aspect of the present invention, a structure that substantially conforms to a given set of atomic coordinates is a structure wherein at least about 75% of such structure has the recited average RMSD value, and in some aspects, at least about 90% of such structure has the recited average RMSD value, and in some aspects, about 100% of such structure has the recited average RMSD value. In particular, the above definition of “substantially conforms” can be extended to include atoms of amino acid side chains. As used herein, the phrase “common amino acid side chains” refers to amino acid side chains that are common to both the structure which substantially conforms to a given set of atomic coordinates and the structure that is actually represented by such atomic coordinates.

In another aspect of the present invention, a three-dimensional structure that substantially conforms to a given set of atomic coordinates is a structure wherein at least about 50% of the common amino acid side chains have an average RMSD of less than about 1.8 Å, and in various aspects, less than about 1.25 Å, and, in other aspects, less than about 1.0 Å, less than about 0.75 Å, less than about 0.5 Å, and less than about 0.25 Å. Inane aspect of the present invention, a structure that substantially conforms to a given set of atomic coordinates is a structure wherein at least about 75% of the common amino acid side chains have the recited average RMSD value, and in some aspects, at least about 90% of the common amino acid side chains have the recited average RMSD value, and in some aspects, about 100% of the common amino acid side chains have the recited average RMSD value.

A three-dimensional structure of a Receptor-Ligand Complex which substantially conforms to a specified set of atomic coordinates can be modeled by a suitable modeling computer program such as MODELER (A. Sali and T. L. Blundell, J. Mol. Biol., vol. 234:779-815, 1993 as implemented in the Insight II software package Insight II, available from Accelerys (San Diego, Calif.)) and those software packages listed in the Examples, using information, for example, derived from the following data: (1) the amino acid sequence of the Receptor-Ligand Complex; (2) the amino acid sequence of the related portion(s) of the protein represented by the specified set of atomic coordinates having a three-dimensional configuration; and, (3) the atomic coordinates of the specified three-dimensional configuration. A three-dimensional structure of a Receptor-Ligand Complex which substantially conforms to a specified set of atomic coordinates can also be calculated by a method such as molecular replacement, which is described in detail below.

A suitable three-dimensional structure of the Receptor-Ligand Complex for use in modeling or calculating the three-dimensional structure of another Receptor-Ligand Complex comprises the set of atomic coordinates represented in Table 1. The set of three-dimensional coordinates set forth in Table 1 is represented in standard Protein Data Bank format. The atomic coordinates have been deposited in the Protein Data Bank, having Accession No. 2BBA. According to the present invention, a Receptor-Ligand Complex has a three-dimensional structure which substantially conforms to the set of atomic coordinates represented by Table 1. As used herein, a three-dimensional structure can also be a most probable, or significant, fit with a set of atomic coordinates. According to the present invention, a most probable or significant fit refers to the fit that a particular Receptor-Ligand Complex has with a set of atomic coordinates derived from that particular Receptor-Ligand Complex. Such atomic coordinates can be derived, for example, from the crystal structure of the protein such as the coordinates determined for the Receptor-Ligand Complex structure provided herein, or from a model of the structure of the protein. For example, the three-dimensional structure of a dimeric protein, including a naturally occurring or recombinantly produced EphB4 receptor protein, substantially conforms to and is a most probable fit, or significant fit, with the atomic coordinates of Table 1. The three-dimensional crystal structure of the Receptor-Ligand Complex may comprise the atomic coordinates of Table 1. Also as an example, the three-dimensional structure of another Receptor-Ligand Complex would be understood by one of skill in the art to substantially conform to the atomic coordinates of Table 1. This definition can be applied to the other EphB4 receptor proteins in a similar manner.

For example, the structure of the EphB4 receptor establishes the general architecture of the EphB receptor family. Accordingly, in some configurations, EphB4 receptor protein sequence homology across eukaryotes can be used as a basis to predict the structure of such receptors, in particular the structure for such receptor-ligand binding sites and other conserved regions.

In various aspects of the present invention, a structure of a Receptor-Ligand Complex substantially conforms to the atomic coordinates represented in Table 1. Such values as listed in Table 1 can be interpreted by one of skill in the art. In other aspects, at three-dimensional structure of a Receptor-Ligand Complex substantially conforms to the three-dimensional coordinates represented in Table 1. In other aspects, a three-dimensional structure of a Receptor-Ligand Complex is a most probable fit with the three-dimensional coordinates represented in Table 1. Methods to determine a substantially conforming and probable fit are within the expertise of skill in the art and are described herein in the Examples section.

A Receptor-Ligand Complex that has a three-dimensional structure which substantially conforms to the atomic coordinates represented by Table 1 includes an EphB4 receptor protein having an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence of a human EphB4 receptor protein, in particular an amino acid sequence having SEQ ID NO:27, across the full-length of the EphB4 receptor sequence. A sequence alignment program such as BLAST (available from the National Institutes of Health Internet web site http://www.ncbi.nlm.nih.gov/BLAST) may be used by one of skill in the art to compare sequences of an EphB receptor to the EphB4 receptor.

A three-dimensional structure of any Receptor-Ligand Complex can be modeled using methods generally known in the art based on information obtained from analysis of a Receptor-Ligand Complex crystal, and from other Receptor-Ligand Complex structures which are derived from a Receptor-Ligand Complex crystal. The Examples section below discloses the production of a Receptor-Ligand Complex crystal, in particular a truncated EphB4 receptor having SEQ ID NO: 2 or 3 complexed with a polypeptide having SEQ ID NO: 1, and a model of a Receptor-Ligand Complex, in particular a truncated EphB4 receptor having SEQ ID NO: 2 or 3 complexed with a polypeptide having SEQ ID NO: 1, using methods generally known in the art based on the information obtained from analysis of a Receptor-Ligand Complex crystal.

An aspect of the present invention comprises using the three-dimensional structure of a crystalline Receptor-Ligand Complex to derive the three-dimensional structure of another Receptor-Ligand Complex. Therefore, the crystalline EphB4 receptor complexed with a ligand, particularly a ligand having a sequence of SEQ ID NO: 1 or SEQ ID NOs: 4 through 26, and the three-dimensional structure of EphB4 complexed with such ligands permits one of ordinary skill in the art to now derive the three-dimensional structure, and models thereof, of any Receptor-Ligand Complex. The derivation of the structure of any Receptor-Ligand Complex can now be achieved even in the absence of having crystal I structure data for such other Receptor-Ligand Complexes, and when the crystal structure of another Receptor-Ligand Complex is available, the modeling of the three-dimensional structure of the new Receptor-Ligand Complex can be refined using the knowledge already gained from the Receptor-Ligand Complex structure.

In some configurations of the present teachings, the absence of crystal structure data for other Receptor-Ligand Complexes, the three-dimensional structures of other Receptor-Ligand Complexes can be modeled, taking into account differences in the amino acid sequence of the other Receptor-Ligand Complex. Moreover, the present invention allows for structure-based drug design of compounds which affect the activity of virtually any EphB receptor, and particularly, of EphB4.

One aspect of the present invention includes a three-dimensional structure of a Receptor-Ligand Complex, in which the atomic coordinates of the Receptor-Ligand Complex are generated by the method comprising: (a) providing an EphB receptor complexed with a ligand in crystalline form; (b) generating an electron-density map of the crystalline EphB receptor complexed with the ligand; and (c) analyzing the electron-density map to produce the atomic coordinates. For example, the structure of human EphB4 receptor in complex with a polypeptide ligand having SEQ ID NO: 1 is provided herein.

Structural Topology of the EphB4 Receptor

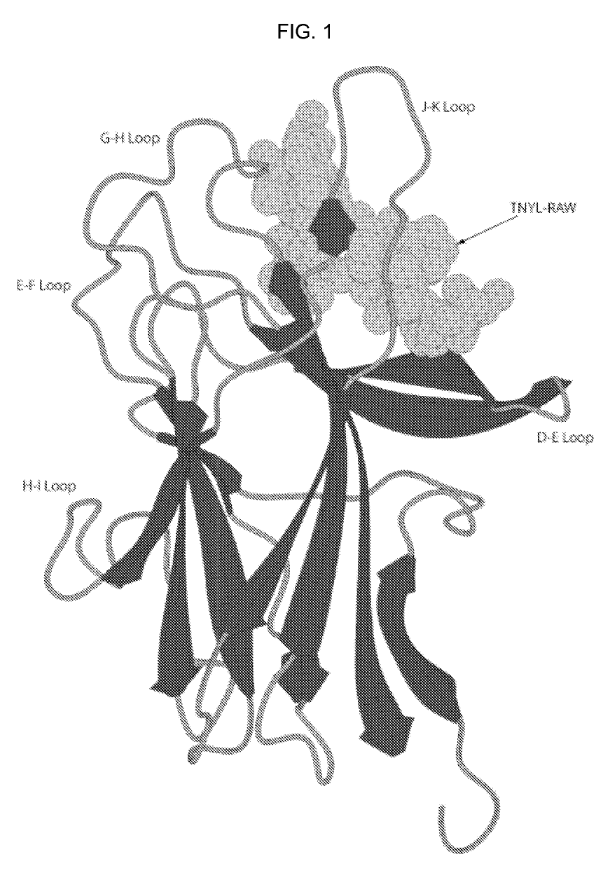

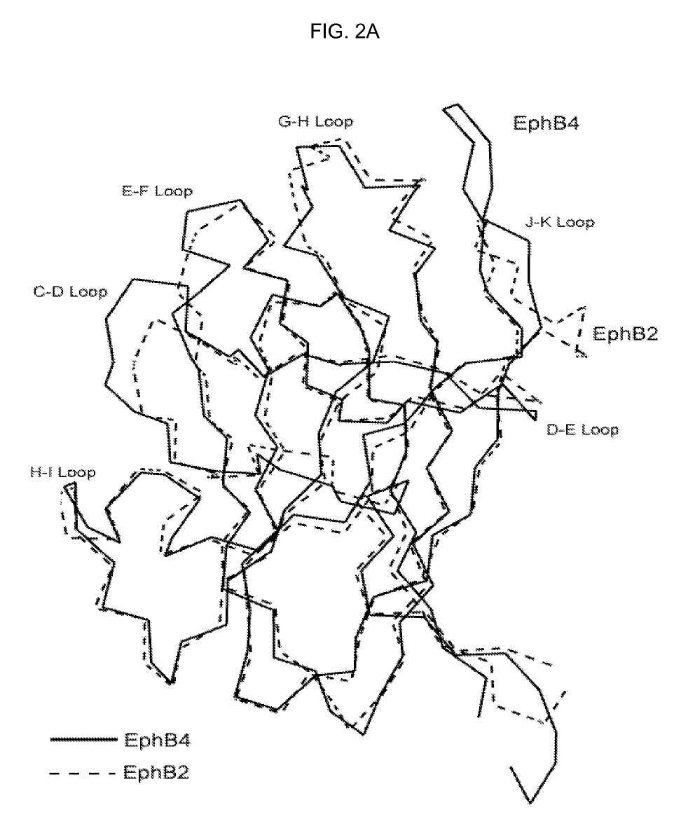

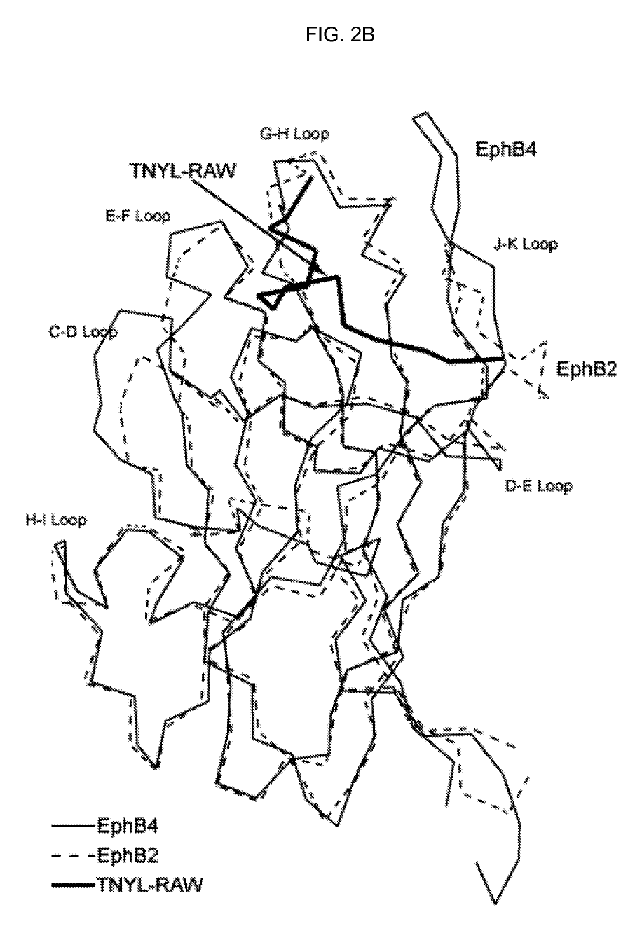

The crystal structure of the human EphB4 ligand binding domain (LBD) in complex with the antagonistic TNYL-RAW peptide (SEQ ID NO: 1) was refined to a 1.65 Å resolution. The structure of the EphB4 receptor is similar to the EphB2 receptor (Himanen et al., 1998), consisting of a jellyroll folding topology composed of 13 anti-parallel β-sheets(FIG. 1) arranged as a compact β-sandwich, with the concave sheet comprised of strands C, F, F′, L, H, and I, and the convex sheet comprised of strands D, E, A, M, G, K, and J (nomenclature according to Himanen et al. (Himanen et al., 1998)). Loops with a varying number of amino acids link each of these β-sheets. The corresponding loops in EphB2 have been shown to play essential roles in receptor-ligand dimerization (D-E, E-F, G-H, J-K) and tetramerization (H-I). Two conserved disulfide bridges that are strictly conserved across Eph receptor subclasses stabilize the G-H loop and the E-F/L-M loops at the top of the β-sandwich. The structure of the globular domain of EphB4 is similar to the apo, ephrin-B2-and ephrin-A5-bound EphB2 structures determined previously (FIG. 2), with root mean square deviations (RMSD) of 1.05, 1.08, and 0.94 Å over equivalent Cα positions (Himanenet al., 2004; Himanen et al., 1998; Himanen et al., 2001).

The ephrin-binding domain of human EphB4 (SEQ ID NO: 27) shares 45% sequence identity with that of human EphB2. Like the EphB2 crystals, the crystals of EphB4 in complex with the TNYL-RAW peptide (SEQ ID NO: 1) contain one molecule in the asymmetric unit. Unlike the apo EphB2 structure, however, the D-E and J-K loops are well ordered in EphB4 and form the peptide binding channel. These loops adopt novel conformations compared to the corresponding loops of the previously described EphB2-ephrin complex structures. Most notably, the J-K loop is significantly shifted in order to avoid steric interference with the peptide (FIG. 2). In fact, this loop is displaced by over 20 Å and17 Å from the furthest Cα positions in the structure of EphB2 in complex with ephrin-B2 orephrin-A5, respectively. The D-E loop is also shifted due to the presence of the antagonist peptide, deviating 2.3 Å and 3.2 Å from the structures of EphB2 in complex with ephrin-B2 and ephrin-A5, respectively. Less significant changes occur at adjacent loops involved in dimerization, due to the new position of the J-K loop, including the disulfide-stabilized G-H loop and the C-D loop, which contains a unique two amino acid insert not found in any other Eph receptors. This insert does not appear to play a role in peptide or ephrin binding and, therefore, presumably does not contribute to the ligand selectivity of EphB4.

EphB4-ephrin-B2 Interaction

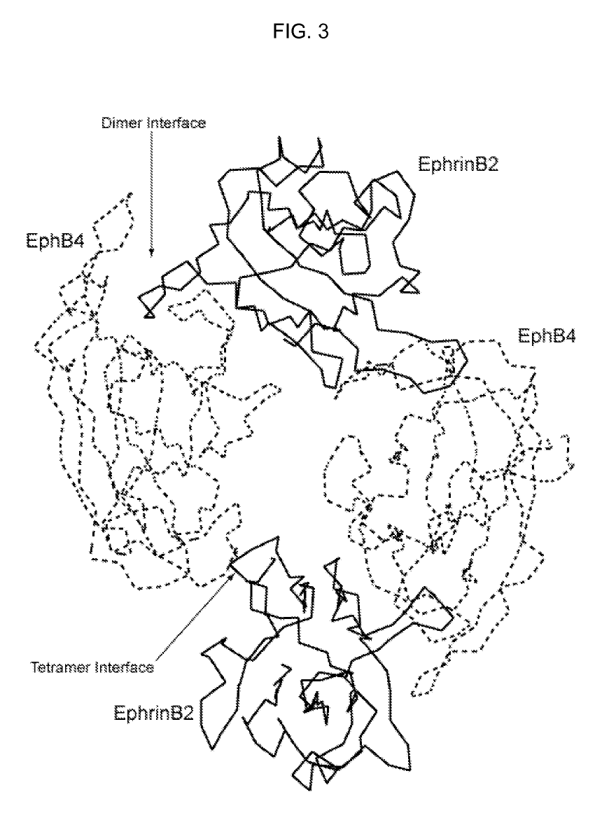

Using the overall topology of the EphB4 binding cleft for comparison, the EphB4-ephrin-B2 interaction was modeled using the EphB2-ephrin-B2 structure as a starting model (FIG. 3). The ephrin-B2 G-H loop forms contacts similar to those described in the structure of the EphB2-ephrin-B2 complex. This high affinity binding interface is highly hydrophobic, and includes residues Phe-120, Pro-122, Leu-124, Trp-125, and Leu-127 of ephrin-B2. The G-H loop of ephrin-B2 is buttressed by the G-H and J-K loops of EphB4, and forms similar main chain hydrogen bonds and numerous van der Waals interactions with EphB4 as previously described in the complex with EphB2 (Himanen et al., 2004; Himanenet al., 2001). In addition, the conserved Cys-61-Cys-184 disulfide bridge of EphB4 is stabilized by Pro-122 from the conserved FSPN segment of the ephrin-B2 G-H loop. As predicted by the Pro-122 positioning in the EphB4 G-H loop, this residue assumes a position similar to that described in the complex with EphB2. The J-K loop of EphB4 shifts towards ephrin-B2 in order to maximize the binding potential between receptor and ligand, as observed in the EphB2-ephrin crystal structures. Indeed, the Eph receptor J-K loop displays remarkable flexibility and is present in a different conformation in EphB2 bound to ephrin-B2or to ephrin-A5. In addition, the J-K loop in the apo structure of EphB2 could not be visualized probably because it is disordered in the absence of a bound ligand (Himanen et al., 2004; Himanen et al., 1998; Himanen et al., 2001).

A second, lower affinity binding interface between EphB2 and ephrin-B2 has been structurally characterized (FIG. 3). This interface, which has been implicated in tetramerization, is absent in the EphB2-ephrinA5 complex, suggesting that it confers subclass binding specificity (Himanen et al., 2004; Himanen et al., 2001). The interface is framed by the H-I subclass-specificity loop. In EphB4, this loop is similar to the EphB2 H-I loop, with a maximum displacement of 2.5 Å at conserved residue Thr-39 of EphB4. Like the EphB2-ephrin-B2 low affinity interface, the EphB4-ephrin-B2 interface is dominated by hydrophobic interactions and few weak polar contacts between the receptor H-I loop and the A-A′ β strands of the ephrin. Hydrophobic interactions similar to those observed in the EphB2-ephrin-B2 complex can also be modeled between the F-G and K-L loops of EphB4and the C-D loop of ephrin-B2.

Accordingly, the present invention provides a three-dimensional structure of the EphB4 receptor protein complexed with a ligand, particularly a polypeptide having SEQ ID NO: 1, can be used to derive a model of the three-dimensional structure of another Receptor-Ligand Complex (i.e., a structure to be modeled). As used herein, a “structure” of a protein refers to the components and the manner of arrangement of the components to constitute the protein. As used herein, the term “model” refers to a representation in a tangible medium of the three-dimensional structure of a protein, polypeptide or peptide. For example, a model can be a representation of the three-dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Accelrys, Inc. (San Diego, Calif.). The phrase “providing a picture of the model” refers to the ability to generate a “hard copy” of the model. Hard copies include both motion and still pictures. Computer screen images and pictures of the model can be visualized in a number of formats including space-filling representations, a-carbon traces, ribbon diagrams and electron density maps.

Suitable target Receptor-Ligand Complex structures to model using a method of the present invention include any EphB receptor protein, polypeptide or peptide that is substantially structurally related to an EphB4 receptor protein complexed with a ligand. In various embodiments, a target Receptor-Ligand Complex structure that is substantially structurally related to an EphB4 receptor protein includes a target Receptor-Ligand Complex structure having an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to an amino acid sequence of a human EphB4 receptor protein, in particular an amino acid sequence having SEQ ID NO: 27, across the full-length of the EphB4 receptor sequence when using, for example, a sequence alignment program such as BLAST (supra). In various aspects of the present invention, target Receptor-Ligand Complex structures to model include proteins comprising amino acid sequences that are at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to amino acid sequence of a truncated EphB4 receptor, EphB4(17-196), having SEQ ID NO: 2 or EphB4 17-198, having SEQ ID NO: 3, when comparing suitable regions of the sequence, such as the amino acid sequence for an ephrin binding site of any one of the amino acid sequences, when using an alignment program such as BLAST (supra) to align the amino acid sequences.

According to the present invention, a structure can be modeled using techniques generally described by, for example, Sali, Current Opinions in Biotechnology, vol. 6, pp. 437-451, 1995, and algorithms can be implemented in program packages such as Insight II, available from Accelerys (San Diego, Calif.). Use of Insight II HOMOLOGY requires an alignment of an amino acid sequence of a known structure having a known three-dimensional structure with an amino acid sequence of a target structure to be modeled. The alignment can be a pairwise alignment or a multiple sequence alignment including other related sequences (for example, using the method generally described by Rost, Meth. Enzymol., vol. 266, pp. 525-539, 1996) to improve accuracy. Structurally conserved regions can be identified by comparing related structural features, or by examining the degree of sequence homology between the known structure and the target structure. Certain coordinates for the target structure are assigned using known structures from the known structure. Coordinates for other regions of the target structure can be generated from fragments obtained from known structures such as those found in the Protein Data Bank. Conformation of side chains of the target structure can be assigned with reference to what is sterically allowable and using a library of rotamers and their frequency of occurrence (as generally described in Ponder and Richards, J. Mol. Biol., vol. 193, pp. 775-791, 1987). The resulting model of the target structure, can be refined by molecular mechanics to ensure that the model is chemically and conformationally reasonable.

Accordingly, one embodiment of the present invention is a method to derive a model of the three-dimensional structure of a target Receptor-Ligand Complex structure the method comprising the steps of: (a) providing an amino acid sequence of a Receptor-Ligand Complex and an amino acid sequence of a target ligand-complexed EphB receptor ;(b) identifying structurally conserved regions shared between the Receptor-Ligand Complex amino acid sequence and the target ligand-complexed EphB4 receptor amino acid sequence; (c) determining atomic coordinates for the target ligand-complexed EphB4 receptor by assigning said structurally conserved regions of the target ligand-complexed EphB4 receptor to a three-dimensional structure using a three-dimensional structure of a Receptor-Ligand Complex based on atomic coordinates that substantially conform to the atomic coordinates represented in Table 1, to derive a model of the three-dimensional structure of the target ligand-complexed EphB4 receptor amino acid sequence. A model according to the present invention has been previously described herein. In one aspect, the model comprises a computer model. The method can further comprise the step of electronically simulating the structural assignments to derive a computer model of the three-dimensional structure of the target ligand-complexed EphB4 receptor amino acid sequence.

Another embodiment of the present invention is a method to derive a computer model of the three-dimensional structure of a target ligand-complexed EphB4 receptor structure for which a crystal has been produced (referred to herein as a “crystallized target structure”). A suitable method to produce such a model includes the method comprising molecular replacement. Methods of molecular replacement are generally known by those of skill in the art and are performed in a software program including, for example, XPLOR available from Accelerys (San Diego, Calif.). In various aspects, a crystallized target ligand-complexed EphB receptor structure useful in a method of molecular replacement according to the present invention has an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the search structure (e.g., human EphB4), when the two amino acid sequences are compared using an alignment program such as BLAST (supra). A suitable search structure of the present invention includes a Receptor-Ligand Complex having a three-dimensional structure that substantially conforms with the atomic coordinates listed in Table 1.

Another aspect of the present invention is a method to determine a three-dimensional structure of a target Receptor-Ligand Complex structure, in which the three-dimensional structure of the target Receptor-Ligand Complex structure is not known. Such a method is useful for identifying structures that are related to the three-dimensional structure of a Receptor-Ligand Complex based only on the three-dimensional structure of the target structure. For example, the present method enables identification of structures that do not have high amino acid identity with an EphB4 receptor protein but which share three-dimensional structure similarities of a ligand-complexed EphB4 receptor. In various aspects of the present invention, a method to determine a three-dimensional structure of a target Receptor-Ligand Complex structure comprises: (a) providing an amino acid sequence of a target structure, wherein the three-dimensional structure of the target structure is not known; (b) analyzing the pattern of folding of the amino acid sequence in a three-dimensional conformation by fold recognition; and (c) comparing the pattern of folding of the target structure amino acid sequence with the three-dimensional structure of a Receptor-Ligand Complex to determine the three-dimensional structure of the target structure, wherein the three-dimensional structure of the Receptor-Ligand Complex substantially conforms to the atomic coordinates represented in Table 1. For example, methods of fold recognition can include the methods generally described in Jones, Curr. Opinion Struc. Biol., vol. 7, pp. 377-387, 1997. Such folding can be analyzed based on hydrophobic and/or hydrophilic properties of a target structure.

One aspect of the present invention includes a three-dimensional computer image of the three-dimensional structure of a Receptor-Ligand Complex. In one aspect, a computer image is created to a structure which substantially conforms with the three-dimensional coordinates listed in Table 1. A computer image of the present invention can be produced using any suitable software program, including, but not limited to, Pymol available from DeLano Scientific, LLC (South San Francisco, Calif.). Suitable computer hardware useful for producing an image of the present invention is known to those of skill in the art.

Another aspect of the present invention relates to a computer-readable medium encoded with a set of three-dimensional coordinates represented in Table 1, wherein, using a graphical display software program, the three-dimensional coordinates create an electronic file that can be visualized on a computer capable of representing said electronic file as a three-dimensional image. Yet another aspect of the present invention relates to a computer-readable medium encoded with a set of three-dimensional coordinates of a three-dimensional structure which substantially conforms to the three-dimensional coordinates represented in Table 1, wherein, using a graphical display software program, the set of three-dimensional coordinates create an electronic file that can be visualized on a computer capable of representing said electronic file as a three-dimensional image. The present invention also includes a three-dimensional model of the three-dimensional structure of a target structure, such a three-dimensional model being produced by the method comprising: (a) providing an amino acid sequences of an EphB4 receptor comprised by a Receptor-Ligand Complex and an amino acid sequence of a target Receptor-Ligand Complex structure; (b) identifying structurally conserved regions shared between the EphB4 receptor amino acid sequence and the amino acid sequence comprised by the target Receptor-Ligand Complex structure; (c) determining atomic coordinates for the target Receptor-Ligand Complex by assigning the structurally conserved regions of the target Receptor-Ligand Complex to a three-dimensional structure using a three-dimensional structure of the EphB4 receptor comprised by a Receptor-Ligand Complex based on atomic coordinates that substantially conform to the atomic coordinates represented in Table 1 to derive a model of the three-dimensional structure of the target Receptor-Ligand Complex. In one aspect, the model comprises a computer model.

Any isolated EphB receptor protein can be used with the methods of the present invention. An isolated EphB receptor protein can be isolated from its natural milieu or produced using recombinant DNA technology (e.g., polymerase chain reaction (PCR) amplification, cloning) or chemical synthesis. To produce recombinant EphB receptor protein, a nucleic acid molecule encoding EphB receptor protein (e.g., SEQ ID NO: 28) can be inserted into any vector capable of delivering the nucleic acid molecule into a host cell. A nucleic acid molecule of the present invention can encode any portion of an EphB receptor protein, in various aspects a full-length EphB receptor protein, and in various aspects a soluble or truncated form of EphB4 receptor protein (i.e., a form of EphB4 receptor protein capable of being secreted by a cell that produces such protein). A suitable nucleic acid molecule to include in a recombinant vector, and particularly in a recombinant molecule, includes a nucleic acid molecule encoding a protein having the amino acid sequence represented by SEQ ID NOs: 2 or 3 and SEQ ID NO: 27.

A recombinant vector can be either RNA or DNA, either prokaryotic or eukaryotic, and typically is a virus or a plasmid. In various aspects, a nucleic acid molecule encoding an EphB4 receptor protein is inserted into a vector comprising an expression vector to form a recombinant molecule. As used herein, an expression vector is a DNA or RNA vector that is capable of transforming a host cell and of affecting expression of a specified nucleic acid molecule. Expression vectors of the present invention include any vectors that function (i.e., direct gene expression) in recombinant cells of the present invention, including in bacterial, fungal, endo parasite, insect, other animal, and plant cells.

An expression vector can be transformed into any suitable host cell to form a recombinant cell. A suitable host cell includes any cell capable of expressing a nucleic acid molecule inserted into the expression vector. For example, a prokaryotic expression vector can be transformed into a bacterial host cell. One method to isolate EphB4 receptor protein useful for producing ligand-complexed EphB4 receptor crystals includes recovery of recombinant proteins from cell cultures of recombinant cells expressing such EphB4 receptor protein.

EphB4 receptor proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, chromatofocusing and differential solubilization. In various aspects of the present invention, an EphB4 receptor protein is purified in such a manner that the protein is purified sufficiently for formation of crystals useful for obtaining information related to the three-dimensional structure of a Receptor-Ligand Complex. In some aspects, a composition of EphB4 receptor protein is about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% pure.

Another embodiment of the present invention includes a composition comprising a Receptor-Ligand Complex in a crystalline form (i.e., Receptor-Ligand Complex crystals). As used herein, the terms “crystalline Receptor-Ligand Complex” and “Receptor-Ligand Complex crystal” both refer to crystallized a Receptor-Ligand Complex and are intended to be used interchangeably. In various aspects of the present invention, a crystalline Receptor-Ligand Complex is produced using the crystal formation method described in the Examples.

In particular, the present invention includes a composition comprising EphB4 receptor complexed with a ligand in a crystalline form (i.e., ligand-complexed EphB4 crystals). As used herein, the terms “crystalline ligand-complexed EphB4” and “ligand complexed EphB4 crystal” both refer to crystallized EphB4 receptor complexed with a ligand and are intended to be used interchangeably. In various aspects of the present invention, a crystal ligand-complexed EphB4 is produced using the crystal formation method described in the Examples. In some aspects, a composition of the present invention includes ligand-complexed EphB4 molecules arranged in a crystalline manner in a space group P41212 so as to form a unit cell of dimensions a=60.97 Å, b=60.97 Å, and c=151.7 Å. A suitable crystal of the present invention provides X-ray diffraction data for determination of atomic coordinates of the ligand-complexed EphB4 to a resolution of about 1.6 Å, and in some aspects about 1.0 Å, and in other aspects at about 0.8 Å.

According to an aspect of the present invention, crystalline Receptor-Ligand Complex can be used to determine the ability of a compound of the present invention to bind to an EphB4 receptor in a manner predicted by a structure based drug design method of the present invention. In various aspects of the present invention, a Receptor-Ligand Complex crystal is soaked in a solution containing a chemical compound of the present invention. Binding of the chemical compound to the crystal is then determined by methods standard in the art.

One aspect of the present invention is a therapeutic composition. A therapeutic composition of the present invention comprises one or more therapeutic compounds. In one aspect, a therapeutic composition is provided that is capable of antagonizing the EphB4 receptor. For example, a therapeutic composition of the present invention can inhibit (i.e., prevent, block) binding of an EphB4 receptor on a cell having anEphB4 receptor (e.g., human cells) to a, e.g., ephrin-B2 or ephrin-B2 analog by interfering with the ligand binding domain of an EphB4 receptor. As used herein, the term “ligand binding domain” refers to the region of a molecule to which another molecule specifically binds.

Suitable inhibitory compounds of the present invention are compounds that interact directly with an EphB receptor protein, and in various aspects an EphB4 receptor protein or truncated EphB4 receptor protein (e.g., SEQ ID NOs: 2 or 3), thereby inhibiting the binding of an EphB4 receptor ligand, e.g., ephrin-B2, to an EphB4 receptor, by blocking the ligand binding domain of an EphB4 receptor (referred to herein as substrate analogs). An EphB4 receptor substrate analog refers to a compound that interacts with (e.g., binds to, associates with, modifies) the ligand binding domain of an EphB4 receptor. An EphB4 receptor substrate analog can, for example, comprise a chemical compound that mimics a polypeptide having SEQ ID NO: 1 or one of SEQ ID NOs: 4 through 26, or that binds specifically to the ephrin binding globular domain of an EphB4 receptor. Further examples of EphB4 receptor substrates upon which an EphB4 ligand analog can be derived are found in U.S. Patent Application No. 20040180823, incorporated herein by reference in its entirety. In various aspects, an EphB4 receptor substrate analog useful in the present invention has an amino acid sequence that is at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 1 or one of SEQ ID NOs: 4 through 26.

According to the present invention, suitable therapeutic compounds of the present invention include peptides or other organic molecules, and inorganic molecules. Suitable organic molecules include small organic molecules. In various aspects, a therapeutic compound of the present invention is not harmful (e.g., toxic) to an animal when such compound is administered to an animal. Peptides refer to a class of compounds that is small in molecular weight and yields two or more amino acids upon hydrolysis. A polypeptide is comprised of two or more peptides. As used herein, a protein is comprised of one or more polypeptides. Suitable therapeutic compounds to design include peptides composed of “L” and/or “D” amino acids that are configured as normal or retro inversopeptides, peptidomimetic compounds, small organic molecules, or homo- or hetero-polymers thereof, in linear or branched configurations.

Therapeutic compounds of the present invention can be designed using structure based drug design. Structure based drug design refers to the use of computer simulation to predict a conformation of a peptide, polypeptide, protein, or conformational interaction between a peptide or polypeptide, and a therapeutic compound. In the present teachings, knowledge of the three-dimensional structure of the EphB4 ligand binding domain of an EphB4 receptor provide one of skill in the art the ability to design a therapeutic compound that binds to EphB4 receptors, is stable and results in inhibition of a biological response, such as tumorigenesis. For example, knowledge of the three-dimensional structure of the EphB4 ligand binding domain of an EphB4 receptor provides to a skilled artisan the ability to design a ligand or an analog of a ligand which can function as a substrate or ligand of an EphB4 receptor.

Suitable structures and models useful for structure-based drug design are disclosed herein. Models of target structures to use in a method of structure-based drug design include models produced by any modeling method disclosed herein, such as, for example, molecular replacement and fold recognition related methods. In some aspects of the present invention, structure based drug design can be applied to a structure of EphB4 in complex with a ligand, particularly a polypeptide having SEQ ID NO: 1, and to a model of a target EphB receptor structure.

One embodiment of the present invention is a method for designing a drug which interferes with an activity of an EphB4 receptor. In various configurations, the method comprises providing a three-dimensional structure of a Receptor-Ligand Complex comprising the EphB4 receptor and at least one ligand of the receptor; and designing a chemical compound which is predicted to bind to the EphB4 receptor. The designing can comprise using physical models, such as, for example, ball-and-stick representations of atoms and bonds, or on a digital computer equipped with molecular modeling software. In some configurations, these methods can further include synthesizing the chemical compound, and evaluating the chemical compound for ability to interfere with an activity of the EphB4 receptor.

Suitable three-dimensional structures of a Receptor-Ligand Complex and models to use with the present method are disclosed herein. According to the present invention, designing a compound can include creating a new chemical compound or searching databases of libraries of known compounds (e.g., a compound listed in a computational screening database containing three-dimensional structures of known compounds). Designing can also include simulating chemical compounds having substitute moieties at certain structural features. In some configurations, designing can include selecting a chemical compound based on a known function of the compound. In some configurations designing can comprise computational screening of one or more databases of compounds in which three-dimensional structures of the compounds are known. In these configurations, a candidate compound can be interacted virtually (e.g., docked, aligned, matched, interfaced) with the three-dimensional structure of a Receptor-Ligand Complex by computer equipped with software such as, for example, the AutoDock software package, (The Scripps Research Institute, La Jolla, Calif.) or described by Humblet and Dunbar, Animal Reports in Medicinal Chemistry, vol. 28, pp. 275-283, 1993, M Venuti, ed., Academic Press. Methods for synthesizing candidate chemical compounds are known to those of skill in the art.

Various other methods of structure-based drug design are disclosed in references such as Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three-dimensional structures and small fragment probes, followed by linking together of favorable probe sites.

In one aspect, a chemical compound of the present invention that binds to the ligand binding domain of a Receptor-Ligand Complex can be a chemical compound having chemical and/or stereochemical complementarity with an EphB receptor, e.g., an EphB4 receptor or ligand such as, for example, a polypeptide having SEQ ID NO: 1. in some configurations, a chemical compound that binds to the ligand binding domain an EphB4 receptor can associate with an affinity of at least about 10-6 M, at least about 10-7 M, or at least about 10-8 M.

Several sites of an EphB4 receptor can be targeted for structure based drug design. These sites include, in non-limiting example residues which contact ephrin-B2 or a polypeptide having SEQ ID NO: 1, e.g., EphB4 D-E and J-K loops; Leu-48, Cys-61, Leu-95, Ser-99 Leu-100, Pro-101, Thr-147, Lys-149, Ala-155, and Cys-184 of SEQ ID NO: 27. Conversely, the structure based drug design can be based upon the sites of the ligand which bind to the EphB receptor, e.g., Phe-120, Pro-122, Leu-124, Trp-125, and Leu-127 of ephrin-B2; and a ligand comprising a sequence motif NxWxL, wherein x is any amino acid.

TNYL-RAW Peptide Binding

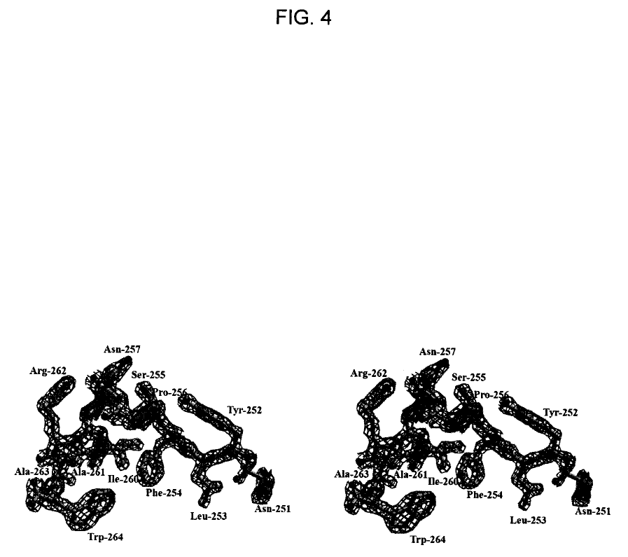

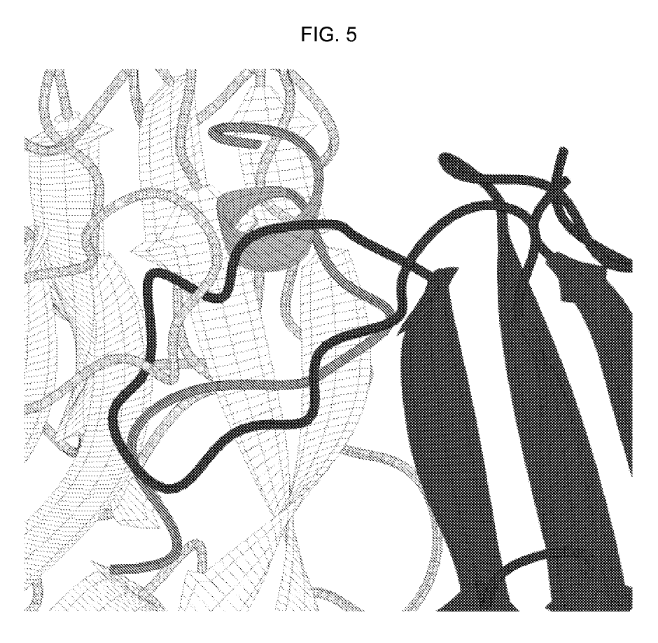

The TNYL-RAW peptide (SEQ ID NO: 1) was modeled into the electron density after initial rounds of refinement using unbiased electron density from simulated annealing omit maps and |Fobs|−|Fcalc|, φcalc maps (FIG. 4). The peptide was inserted into the same cleft occupied by ephrin-B2, along the hydrophobic upper convex portion of the EphB4 receptor, which is situated on top of a β-sheet “floor” formed by β-strands D and E. In addition, loops D-E, E-F, G-H and J-K effectively buttress the peptide in the cavity, forming numerous van der Waals interactions and main chain hydrogen bonds that stabilize binding. Although the TNYL-RAW peptide (SEQ ID NO: 1) shares the FSPN sequence motif with the G-H loop of ephrin-B2 (Koolpe et al., 2005), surprisingly it assumes a distinct conformation as compared with the ephrin-B2 G-H loop bound to EphB2 (Himanen et al., 2001) (FIG. 5). The peptide has little secondary structure at the N terminus, but forms a pseudo-helix at its C-terminal end. A glycine-proline motif in the middle of the peptide induces a sharp 90° turn that angles the peptide into the upper edge of the binding cleft adjacent to the EphB4 G-H loop, where the high affinity-conferring RAW sequence binds.

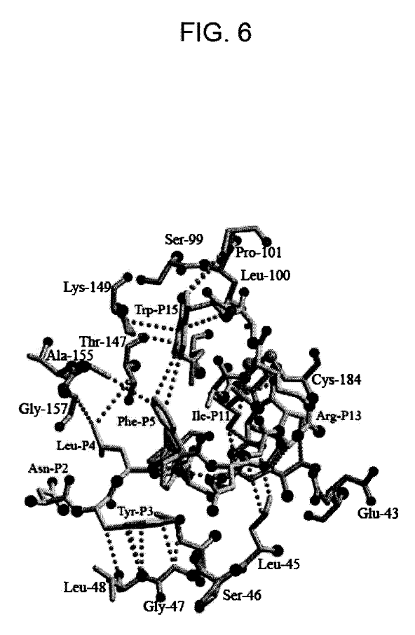

The N-terminal residue of the peptide, Thr-P1, could not be modeled into the electron density map, and therefore is not depicted in the final model of the complex. The adjacent Asn-P2 is located along the plane of β-strand D of EphB4, in between the D-E and J-K loops, and forms few interactions with the receptor (FIG. 1). The position of Asn-P2 suggests that the N-terminal Thr-P1 may be disordered because it does not bind to EphB4, likely explaining its absence in the electron density map. Tyr-P3 is positioned on top of β-sheet D, forming a pseudo-sandwich between the D-E loop of the EphB4 receptor and the N-terminal end of the peptide. The hydroxyl group of the tyrosine forms a hydrogen bond with the EphB4 main chain oxygen of Ser-39 and stacking interactions with neighboring β-sheet residues (FIG. 6). Similar interactions are observed between Trp-125 of ephrin-B2 and the EphB2 D-E loop. These interactions likely play a key role in ordering this region of the bound ephrin (Himanen et al., 2004; Himanen et al., 2001).

The G-H loop of ephrin-B2 contains a conserved FSPN sequence, which plays an essential role in receptor binding and is the only sequence within the G-H loop that is also present in the TNYL-RAW peptide (SEQ ID NO: 1). Substantial hydrophobic interactions between this sequence and the G-H loop of the EphB2 receptor essentially lockephrin-B2 into the binding cleft of the receptor (Himanen et al., 2001). In the structure of the peptide in complex with EphB4, the corresponding Phe-P5 of the peptide is completely buried by the J-K loop of the receptor and by residues of the peptide, including lie-P11 and Trp-P15. This residue is situated more than 8 Å away from the equivalent phenylalanine residue in the ephrin-B2 G-H loop, and the N- to C-terminal orientation of the FSPN sequence in the ephrin and the peptide are pointed in opposite directions. Furthermore, unlike the SPN sequence of ephrin-B2 in complex with EphB2, the SPN sequence of the peptide is not buried by the hydrophobic G-H loop of EphB4, but instead is positioned along the solvent exposed surface of the receptor. The side chain of Ser-P6 forms a hydrogen bond with the main chain nitrogen of Asn-P8, which together with the intervening Pro-P7contributes to a sharp turn in the middle of the peptide. This turn positions Ile-P11 to interact with the conserved disulfide bridge in the E-F and L-M loops of EphB4 (Cys-61-Cys-184). Ile-P11 resides in the equivalent position as the conserved Pro-122 in ephrin-B2 (Pro-125 in ephrin-A5), which interacts with the corresponding disulfide bridge (Cys-60-Cys-192) in EphB2. The side chain of Ile-P11 forms a frame similar to the ephrin-B2 Pro-122 CD, CG, and CB positions, thus providing a hydrophobic backbone that stabilizes the position of the functionally important disulfide bridge in EphB4.

Alignment of a number of the EphB4-binding peptides that were identified by phage display (e.g., SEQ ID NOs: 4 through 26) revealed a conserved glycine-proline motif corresponding to a tryptophan located at the tip of the ephrin-B2 G-H loop. Although praline and tryptophan are not structurally similar, the G-P residues in the peptides were predicted to mimic the turn of the middle of the ephrin G-H loop (Koolpe et al., 2005). Surprisingly, the bend induced by the G-P motif is instead most similar to the turn present at the beginning of the ephrin-B2 G-H loop and formed by residues Phe-117, Gln-118 and Glu-119, which angle the ephrin G-H loop into the hydrophobic cleft of the Eph receptor. The G-P turn in the TNYL-RAW peptide (SEQ ID NO: 1) positions the RAW sequence into the upper edge of the EphB4 binding cleft, where Trp-P15 is effectively stabilized between the J-K and G-H loops of the receptor. Trp-P15 forms a main chain hydrogen bond with Ser-93 and hydrophobic interactions with Leu-88, Leu-93, Pro-94, Lys-142, and Phe-P5. These interactions are similar to those formed by Phe-120 in the ephrin-B2 FSPN motif. Unlike Phe-120 of ephrin-B2, however, Trp-P15 is buried within the hydrophobic binding cleft maximizing its interactions with the receptor. Arg-P13, which is also part of the peptide sequence important for high affinity binding, forms a hydrogen bond with the sidechain of Glu-43 of the receptor, and also aids in structuring the C-terminal end of the peptide by forming a side-chain to main-chain hydrogen bond with the solvent exposed Asn-P8. Together, Arg-P13 and Trp-P15 could disrupt several hydrogen bonds in the high affinity dimerization interface between EphB4 and the ephrin-B2 ligand, consistent with the antagonistic properties of the TNYLRAW peptide (SEQ ID NO: 1). Overall, the network of interactions between EphB4 and the high affinity-conferring RAW sequence is highly stable and similar to the interactions of the conserved FSPN sequence of ephrin-B2. Taken together, these data suggest that the TNYL-RAW peptide (SEQ ID NO: 1) can inhibit ephrin binding to the high affinity dimerization interface of the EphB4 ephrin-binding domain (FIG. 6).

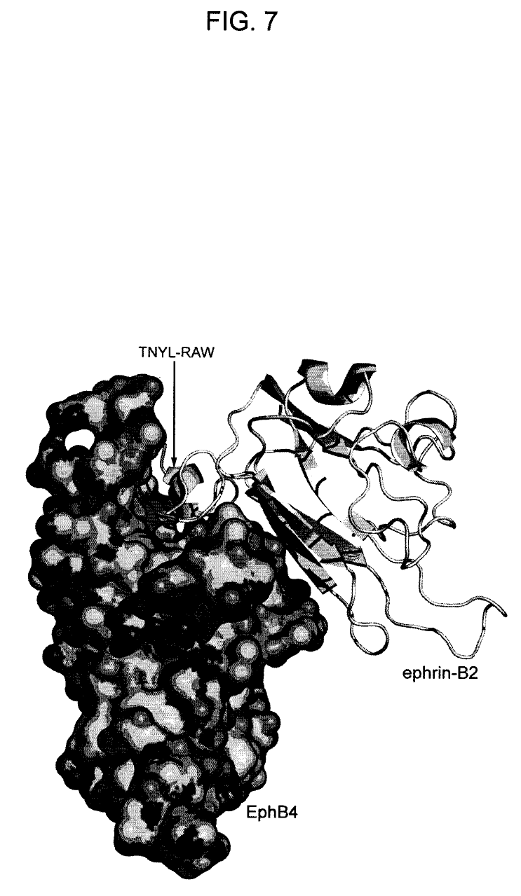

A second region of the dimerization interface has been characterized adjacent to the high affinity dimerization interface that provides significant structural integrity for complex formation (FIG. 7), with numerous hydrogen bonds formed between receptor and ephrin from backbone-backbone, backbone-sidechain, and sidechain-sidechain contacts (Himanen et al., 2001). Several of these contacts can also be mapped onto a model of the EphB4-ephrin-B2 complex, including interactions between Asp-110 (L) and Thr-38 (R), Leu-101 (L) and Ser-47 (R) and Lys-112 (L) and Ser55 (R). The residues in this second interface of EphB4 would remain accessible to ephrin-B2 in the presence of the bound TNYL-RAW peptide (SEQ ID NO: 1). However, the protruding Arg-P13 and Asn-P8 of the peptide would sterically interfere with the positioning of β-strand G of ephrin-B2. Arg-P13 in particular extends away from the body of the peptide into the space that would be occupied by β-strand G of ephrin-B2. Therefore, the presence of the bound peptide would likely reposition the ephrin such that weak hydrogen bonds would dominate this affinity interface, making the interaction much weaker. In addition, the FSPN residues of the peptide would sterically clash with several residues at the tip of the low affinity EphB4-ephrin-B2 interface, including residues Lys-116, Phe-117, and Gln-118 of the ephrin ligand.

Thermodynamic Characterization

The molecular determinants were experimentally verified for the high affinity binding of the peptide predicted based on the crystal structure, a thermodynamic characterization of TNYL-RAW and truncated forms of this peptide using isothermal titration calorimetry (ITC). The binding of TNYL-RAW (SEQ ID NO: 1) to the human EphB4 ephrin-binding domain (amino acids 17-196; SEQ ID NO: 2) at 25° C. yields a Kd of 70 nM and a ΔHo of −14.7 kcal mol-1 (Table 1 B). As an internal control, the interaction between EphB4 (17-196) and the Eph-binding domain of human ephrin-B2 yielded a Kd of 40 nM and a βHo of +3.3 kcal mol-1. This is slightly lower than the affinity reported for the interaction between the entire mouse EphB4 extracellular domain and mouse or human ephrin-B2 (Table 1A). The existence of a third low affinity Eph-ephrin interface located outside the ephrin-binding domain provides for the difference (Smith et al., 2004).

The structural information suggests that two contact areas between EphB4 and the peptide are particularly critical for their interaction. One involves the N-terminal Tyr-P3 (TNYL) and the other the C-terminal Arg-P13 and Trp-P15 (RAW). The importance of these residues was verified by determining the Kd values for binding of peptides with N- and C-terminal truncations to human EphB4 (17-196) as measured in ITC experiments (Table 1B). Deletion of the N-terminal Thr-P1 and Asn-P2 of the peptide produced negligible changes in Kd (65-80 nM) and ΔHo. However, deletion of Tyr-P3 caused a 40-fold reduction in affinity (Kd=3.5 μM), indicating that the tyrosine is the first residue from the N-terminus of the peptide that is required for high affinity binding. The RAW sequence is predicted to play an essential role in peptide binding due to its extensive interactions with EphB4 residues in the EphB4-peptide complex structure. Truncation of this sequence indeed resulted in very weak binding (Kd>140 μM), in agreement with previous results (Koople et al., 2005), indicating that this region of the peptide provides critical binding determinants. Trp-P15 in particular is highly stabilized by both polar and hydrophobic interactions with the same region of EphB4 that is modeled to interact with the conserved FSPN sequence of ephrin-B2.

Competition studies measuring the ability of truncated forms of the TNYL-RAW peptide (SEQ ID NO: 1) to antagonize murine ephrin-B2 binding to murine EphB4 are also provided in addition to the ITC results with the human proteins. Thr-P1 and Asn-P2 do not affect the ability of TNYL-RAW to inhibit ephrin-B2 binding to EphB4 (Table 1A). In contrast, Tyr-P3 was required for efficient antagonistic properties. The IC50 for inhibition of ephrin-B2 binding to the TNYL-RAW and YL-RAW (NYLFSPNGPIARAW; SEQ IN NO: 30) peptide is approximately 40 nM and that for L-RAW (YLFSPNGPIARAW; SEQ ID NO: 31) is approximately 15 μM (Table 1A).

EphB4 is the sole member of the Eph receptor family that interacts preferentially with only one ephrin ligand, ephrin-B2, whereas it is only weakly activated by ephrin-B1 and ephrin-B3, the other two ephrins of the B subclass. EphB2, on the other hand, is activated by multiple ephrins, including one from the A subclass (Himanen et al., 2004). The overall structure of the EphB4 ephrin-binding domain is similar to that previously reported for EphB2 (Himanen et al., 2004; Himanen et al., 1998; Himanen et al., 2001). Furthermore, the overall topology of the high affinity dimerization interface is remarkably similar between the EphB2 and EphB4 structures, considering that only 42% of the residues in the EphB4 binding cleft are identical to the corresponding residues of EphB2 (Koolpe et al., 2005). However, there are important differences that could explain the higher ligands electivity of EphB4.

Several amino acid residues that make important contacts with the ephrin G-H loop in the high affinity dimerization interface of EphB2 are not conserved in EphB4. For example, Ser-194 of EphB2 is conserved in other EphB receptors but not in EphB4, where an alanine is present at the corresponding position. Therefore, EphB4 cannot form the polar interaction observed between the side chain of Ser-194 of EphB2 and the ephrin-B2 main chain oxygen of Glu-128. Furthermore, all EphB receptors have an aromatic residue at the position corresponding to Tyr-57 of EphB2. In EphB4 this position is occupied by Leu (residue 48), which cannot form a hydrogen bond with the main chain oxygen of Pro-150 of ephrin-B2 or an aromatic-aromatic interaction with Phe-113 of ephrin-B2, as observed for Tyr-57 of EphB2. Rather, Leu-48 forms only weak hydrophobic interactions with ephrin-B2. Leu-95 is present in EphB4 at the corresponding Arg-103 position of EphB2, resulting in the absence of another salt bridge that is present in the dimerization interface of EphB2 with both ephrin-B2 and ephrin-A5 (Himanen et al., 2004; Himanen et al., 2001). The presence of a leucine is unique to EphB4, because an arginine is conserved at this position in all other Eph receptors across subclasses.

Some of the differences between EphB4 and the other EphB receptors also explain the ability of the TNYL-RAW peptide (SEQ ID NO: 1) to selectively bind only to EphB4. In particular, two non-conserved amino acids of EphB4 make critical contacts with the high affinity-conferring RAW motif in the peptide. Leu-95 of EphB4 forms van der Waals interactions with both Phe-P3 and Trp-P15 of the peptide, aiding in the overall positioning of the peptide. The arginine present in the corresponding position of all other Eph receptors (Arg-103 in EphB2, see above) would result in steric clashes with both Trp-P15 and Phe-P5in the EphB4-TNYL-RAW structure. Furthermore, Thr-147 of EphB4 forms hydrophobic interactions with several residues of the peptide and aids in the overall positioning of Phe-P5from the peptide. The phenylalanine present in the corresponding position of other Eph receptors (Phe-155 in EphB2) would instead result in a steric clash with Phe-P5 of the peptide. The non-conserved Leu-48 of EphB4 also contributes to peptide binding by forming a van der Waals interaction with the tyrosine in the TNYL-RAW peptide (SEQ ID NO: 1).

Additional differences in the lower affinity tetramer interface of EphB4 and other EphB receptors may further contribute to the selectivity of EphB4 for ephrin-B2. For example, EphB4 lacks several residues involved in interactions that provide stability in the EphB2-ephrin-B2 tetrameric complex. Of particular interest is the absence of the stacking interaction between Phe-128 (EphB2) and Tyr-37 (ephrin-B2), due to the presence of an alanine (Ala-120) at the equivalent position in EphB4. An alanine at this position should result in a substantial loss of stability at the tetramer interface due not only to the absence of the stacking interaction with the ephrin aromatic residue, but also to the absence of interactions with residues Ser-139, Gly-141, and Asn-142 of ephrin-B2. Interestingly, ephrin-B1 contains a serine at the position corresponding to Tyr-37 in ephrin-B2, which is also predicted to destabilize the tetramer interface (Nikolov et al., 2005). In association with the missing aromatic in EphB4 (Phe-128) at the tetramer interface, formation of an EphB4-ephrin-B1 tetramer is highly unfavorable, providing one explanation for the weak interaction between this receptor and ligand. In addition, the presence in EphB4 of Thr-127 instead of Phe-135 of EphB2 results in the absence of the hydrophobic interaction with Glu-134 of ephrin-B2, which is not replaced by other interactions with the ephrin. Despite the weaker contacts at the tetramer interface, we have found that the EphB4 receptor can form a heterotetramer with the ephrin-B2 ligand (data not shown).

An interesting feature of the Eph receptors is the flexibility of their D-E and J-K loops, which line the high affinity ephrin binding cleft (Himanen et al., 2004; Himanen et al., 2001). These loops are disordered in the apo structure of EphB2, suggesting that a ligand is required to promote their stability. EphB2 can accommodate ephrins of both the A and B subclasses by shifting the position of the J-K loop by more than 10 Å. Furthermore in the structures of EphB2 in complex with ephrin-B2 or ephrin-A5, the J-K loop is positioned adjacent to the D-E loop, forming weak hydrophobic interactions that likely aid in the ordering of these loops. In the presence of bound TNYL-RAW peptide (SEQ ID NO: 1), the J-K loop of EphB4 is shifted by as much as 20 Å compared to the J-K loop of apo EphB2, suggesting that this region can undergo marked movements in order to accommodate a ligand. Supporting the idea that a ligand stabilizes the conformation of the Eph receptor ephrin-binding domain, EphB4 readily formed well-diffracting crystals in the presence of the TNYL-RAW peptide (SEQ ID NO: 1), whereas the apo form of the receptor did not crystallize.

The topology of the high affinity binding cleft in the complex with the TNYL-RAW peptide (SEQ ID NO: 1) can also accommodate the modeled ephrin-B2 G-H loop. Thus, despite marked differences in the primary and secondary structures of the peptide and the ephrin G-H loop, the two ligands both similarly fit in the EphB4 binding cleft. It will be interesting to model the many other EphB4-specific peptides that were identified by phage display (Koolpe et al., 2005) in order to gain information on the range of residues that can be accommodated at each position, as well as additional ligand structures that can be accommodated by the ephrin binding cleft of EphB4. Two of the peptides identified by phage display are unrelated in sequence to TNYL-RAW, but share with ephrin-B2 the sequence motif NxWxL (where x is any amino acid). Several other peptides with different sequences also appear to target the ephrin binding cleft of EphB4.

Although the precise roles of Eph receptor-ephrin bi-directional signaling in angiogenesis are incompletely understood, it is clear that the EphB4 receptor has a critical function because it is required for normal vascular development in the embryo (Gerety et al., 1999). The ability to modulate EphB4-ephrin-B2 binding will be critical to dissect the roles of these molecules in tumorigenesis and angiogenesis. Furthermore, antagonizing EphB4-ephrin-B2 binding will undoubtedly be of high therapeutic value. High affinity selective antagonists of this interaction could be used to inhibit tumor angiogenesis (Martiny-Baron et al., 2004; Noren et al., 2004) and pathological forms of angiogenesis, including inflammatory angiogenesis and the excessive retinal neovascularization that plays an important role in retinopathy of prematurity, macular degeneration, and diabetic retinopathy (Yuan et al., 2004; Zamora et al., 2005). The high resolution structure of the ephrin-binding domain of EphB4 in complex with a highly selective and potent peptide antagonist, which we report here, will allow the design of novel compounds that recapitulate the critical contacts of the peptide with EphB4 while having good pharmacokinetic properties.

Drug design strategies as specifically described above with regard to residues and regions of the ligand-complexed EphB4 receptor crystal can be similarly applied to the other EphB structures, including other EphB receptors disclosed herein. One of ordinary skill in the art, using the art recognized modeling programs and drug design methods, many of which are described herein, can modify the EphB4 design strategy according to differences in amino acid sequence. For example, this strategy can be used to design compounds which regulate a function of the EphB4 receptor in EphB receptors. In addition, one of skill in the art can use lead compound structures derived from one Eph-B receptor, such as the EphB4 receptor, and take into account differences in amino acid residues in other EphB4 receptors.

In the present method of structure-based drug design, it is not necessary to align a candidate chemical compound (i.e., a chemical compound being analyzed in, for example, a computational screening method of the present invention) to each residue in a target site. Suitable candidate chemical compounds can align to a subset of residues described for a target site. In some configurations of the present invention, a candidate chemical compound can comprise a conformation that promotes the formation of covalent or non-covalent crosslinking between the target site and the candidate chemical compound. In certain aspects, a candidate chemical compound can bind to a surface adjacent to a target site to provide an additional site of interaction in a complex. For example, when designing an antagonist (i.e., a chemical compound that inhibits the binding of a ligand to an EphB4 receptor by blocking a ligand binding domain or interface), the antagonist can be designed to bind with sufficient affinity to the binding site or to substantially prohibit a ligand from binding to a target area. It will be appreciated by one of skill in the art that it is not necessary that the complementarity between a candidate chemical compound and a target site extend overall residues specified here.

In various aspects, the design of a chemical compound possessing stereochemical complementarity can be accomplished by means of techniques that optimize, chemically or geometrically, the “fit” between a chemical compound and a target site. Such techniques are disclosed by, for example, Sheridan and Venkataraghavan, Acc. Chem Res., vol. 20, p. 322, 1987: Goodford, J. Med. Chem., vol. 27, p. 557, 1984; Beddell, Chem. Soc. Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

Some embodiments of the present invention for structure-based drug design comprise methods of identifying a chemical compound that complements the shape of an EphB4 receptor, particularly one that substantially conforms to the atomic coordinates of Table 1, or a structure that is related to an EphB4 receptor. Such method is referred to herein as a “geometric approach”. In a geometric approach of the present invention, the number of internal degrees of freedom (and the corresponding local minima in the molecular conformation space) can be reduced by considering only the geometric (hard-sphere) interactions of two rigid bodies, where one body (the active site) contains “pockets” or “grooves” that form binding sites for the second body (the complementing molecule, such as a ligand).

The geometric approach is described by Kuntz et al., J. Mol. Biol., vol. 161, p. 269, 1982, which is incorporated by this reference herein in its entirety. The algorithm for chemical compound design can be implemented using a software program such as AutoDock, available from The Scripps Research Institute (La Jolla, Calif.). One or more extant databases of crystallographic data (e.g., the Cambridge Structural Database System maintained by University Chemical Laboratory, Cambridge University, Lensfield Road, Cambridge CB2 IEW, U.K. or the Protein Data Bank maintained by Rutgers University) can then be searched for chemical compounds that approximate the shape thus defined. Chemical compounds identified by the geometric approach can be modified to satisfy criteria associated with chemical complementarity, such as hydrogen bonding, ionic interactions or Van der Waals interactions.

In some embodiments, a therapeutic composition of the present invention can comprise one or more therapeutic compounds. A therapeutic composition can further comprise other compounds capable of inhibiting an EphB4 receptor. A therapeutic composition of the present invention can be used to treat disease in an animal such as, for example, a human in need of treatment by administering such composition to the human. Non-limiting examples of animals to treat include mammals, reptiles and birds, companion animals, food animals, zoo animals and other economically relevant animals (e.g., racehorses and animals valued for their coats, such as minks). Additional animals to treat include dogs, cats, horses, cattle, sheep, swine, chickens, turkeys. Accordingly, in some aspects, animals to treat include humans.

A therapeutic composition of the present invention can also include an excipient, an adjuvant and/or carrier. Suitable excipients include compounds that the animal to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal, o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, a therapeutic composition can include a carrier. Carriers include compounds that increase the half-life of a therapeutic composition in the treated animal. Suitable carriers include, but are not limited to, polymeric controlled release vehicles, biodegradable implants, liposomes, bacteria, viruses, other cells, oils, esters, and glycols.

Acceptable protocols to administer therapeutic compositions of the present invention in an effective manner include individual dose size, number of doses, frequency of dose administration, and mode of administration. Determination of such protocols can be accomplished by those skilled in the art. Modes of administration can include, but are not limited to, subcutaneous, intradermal, intravenous, intranasal, oral, transdermal, intraocular and intramuscular routes.

In yet another embodiment, a method is provided for assaying EphB4 receptor binding to a compound. The method can comprise providing an EphB4 receptor bound with a polypeptide, e.g., having SEQ ID NO: 1, followed by contacting the ligand bound EphB4 receptor with a compound. The release can be detected indicating that the compound binds to the EphB4 receptor. The EphB4 receptor can be a polypeptide having SEQ ID NO: 2 or 3. In certain embodiments, the EphB4 receptor can consist essentially of EphB4 D-E and J-K loops. The EphB4 receptor can also consist essentially of Leu-48, Cys-61, Leu-95, Ser-99 Leu-100, Pro-101, Thr-147, Lys-149, Ala-155, and Cys-184 of EphB4 (SEQ ID NO: 27). The EphB4 receptor can be a human EphB4 receptor.

In another embodiment, a method is provided for crystallizing an EphB4 receptor which includes providing an EphB4 receptor in contact with a first polypeptide having SEQ ID NO: 1, followed by contacting the EphB4 receptor in contact with the first polypeptide with a second polypeptide having at least 50% sequence identity to SEQ ID NO:1, but not identical to SEQ ID NO: 1, wherein the EphB4 receptor in contact with the first and second polypeptides forms an EphB4 receptor crystal. The second polypeptide can comprise at least 75% sequence identity to SEQ ID NO: 1, and in certain embodiments, at least 90% sequence identity to SEQ ID NO: 1.

In yet another embodiment, a method is provided for crystallizing an EphB4 receptor which includes providing an EphB4 receptor in contact with a polypeptide having SEQ ID NO: 1, followed by contacting the EphB4 receptor in contact with the polypeptide with a therapeutic compound as provided above, wherein the EphB4 receptor in contact with the polypeptide and the compound forms an EphB4 receptor crystal.

In another embodiment, a composition is provided comprising EphB4 receptor, a ligand, and a therapeutic compound as provided above. The EphB4 receptor can be a polypeptide having SEQ ID NO: 2 or 3. The EphB4 receptor can also consist essentially of EphB4 D-E and J-K loops or Leu-48, Cys-61, Leu-95, Ser-99 Leu-100, Pro-101, Thr-147, Lys-149, Ala-155, and Cys-184 of SEQ ID NO: 27. In certain embodiments, the EphB4 receptor can be a human EphB4 receptor.

In certain embodiments, the ligand can be a polypeptide having SEQ ID NO: 1 or polypeptides having SEQ ID NO: 4 through SEQ ID NO: 26. In other embodiments, the ligand can be a polypeptide having at least 50%, 75% or 90% sequence identity to a polypeptide selected from the group consisting of polypeptides having SEQ ID NO: 1 and SEQ ID NO: 4 through SEQ ID NO: 26.

TABLE 1
Protein Databank Coordinates
of Eph4 Receptor-Complexed TNYL-RAW

HEADER ----- XX-XXX-XX xxxx
COMPND ---
REMARK 3
REMARK 3 REFINEMENT.
REMARK 3 PROGRAM: REFMAC 5.2.0005
REMARK 3 AUTHORS: MURSHUDOV, VAGIN, DODSON
REMARK 3
REMARK 3 REFINEMENT TARGET: MAXIMUM LIKELIHOOD
REMARK 3
REMARK 3 DATA USED IN REFINEMENT.
REMARK 3 RESOLUTION RANGE HIGH (ANGSTROMS): 1.65
REMARK 3 RESOLUTION RANGE LOW (ANGSTROMS): 56.61
REMARK 3 DATA CUTOFF (SIGMA(F)): NONE
REMARK 3 COMPLETENESS FOR RANGE (%): 99.92
REMARK 3 NUMBER OF REFLECTIONS: 31786
REMARK 3
REMARK 3 FIT TO DATA USED IN REFINEMENT.
REMARK 3 CROSS-VALIDATION METHOD: THROUGHOUT
REMARK 3 FREE R VALUE TEST SET SELECTION: RANDOM
REMARK 3 R VALUE (WORKING + TEST SET): 0.16824
REMARK 3 R VALUE (WORKING SET): 0.16622
REMARK 3 FREE R VALUE: 0.18595
REMARK 3 FREE R VALUE TEST SET SIZE (%): 10.0
REMARK 3 FREE R VALUE TEST SET COUNT: 3533
REMARK 3
REMARK 3 FIT IN THE HIGHEST RESOLUTION BIN.
REMARK 3 TOTAL NUMBER OF BINS USED: 20
REMARK 3 BIN RESOLUTION RANGE HIGH: 1.650
REMARK 3 BIN RESOLUTION RANGE LOW: 1.693
REMARK 3 REFLECTION IN BIN (WORKING SET): 2286
REMARK 3 BIN COMPLETENESS (WORKING + TEST) (%): 98.94
REMARK 3 BIN R VALUE (WORKING SET): 0.156
REMARK 3 BIN FREE R VALUE SET COUNT: 243
REMARK 3 BIN FREE R VALUE: 0.195
REMARK 3
REMARK 3 NUMBER OF NON-HYDROGEN ATOMS USED IN
REFINEMENT.
REMARK 3 ALL ATOMS: 1843
REMARK 3
REMARK 3 B VALUES.
REMARK 3 FROM WILSON PLOT (A**2): NULL
REMARK 3 MEAN B VALUE (OVERALL, A**2): 11.757
REMARK 3 OVERALL ANISOTROPIC B VALUE.
REMARK 3 B11 (A**2): 0.21
REMARK 3 B22 (A**2): 0.21
REMARK 3 B33 (A**2): −0.42
REMARK 3 B12 (A**2): 0.00
REMARK 3 B13 (A**2): 0.00
REMARK 3 B23 (A**2): 0.00
REMARK 3
REMARK 3 ESTIMATED OVERALL COORDINATE ERROR.
REMARK 3 ESU BASED ON R VALUE (A): 0.078
REMARK 3 ESU BASED ON FREE R VALUE (A): 0.077
REMARK 3 ESU BASED ON MAXIMUM LIKELIHOOD (A): 0.041
REMARK 3 ESU FOR B VALUES BASED ON MAXIMUM LIKELIHOOD
(A**2): 2.276
REMARK 3
REMARK 3 CORRELATION COEFFICIENTS.
REMARK 3 CORRELATION COEFFICIENTS FO-FC: 0.958
REMARK 3 CORRELATION COEFFICIENTS FO-FC FREE: 0.952
REMARK 3
REMARK 3 RMS DEVIATIONS FROM IDEAL VALUES COUNT RMS
WEIGHT
REMARK 3 BOND LENGTHS REFINED ATOMS (A): 1667; 0.017;
0.021
REMARK 3 BOND LENGTHS OTHERS (A): 1438; 0.002; 0.020
REMARK 3 BOND ANGLES REFINED ATOMS (DEGREES): 2282;
1.801; 1.959
REMARK 3 BOND ANGLES OTHERS (DEGREES): 3327; 0.894;
3.000
REMARK 3 TORSION ANGLES, PERIOD 1 (DEGREES): 201;
7.448; 5.000
REMARK 3 TORSION ANGLES, PERIOD 2 (DEGREES): 72;
31.759; 23.056
REMARK 3 TORSION ANGLES, PERIOD 3 (DEGREES): 242;
10.377; 15.000
REMARK 3 TORSION ANGLES, PERIOD 4 (DEGREES): 9; 16.603;
15.000
REMARK 3 CHIRAL-CENTER RESTRAINTS (A**3): 246; 0.117;
0.200
REMARK 3 GENERAL PLANES REFINED ATOMS (A): 1823;
0.010; 0.020
REMARK 3 GENERAL PLANES OTHERS (A): 348; 0.001;
0.020
REMARK 3 NON-BONDED CONTACTS REFINED ATOMS (A): 244;
0.277; 0.200
REMARK 3 NON-BONDED CONTACTS OTHERS (A): 1343; 0.212;
0.200
REMARK 3 NON-BONDED TORSION REFINED ATOMS (A): 744;
0.182; 0.200
REMARK 3 NON-BONDED TORSION OTHERS (A): 881; 0.88;
0.200
REMARK 3 H-BOND (X . . . Y) REFINED ATOMS (A): 144; 0.156;
0.200
REMARK 3 H-BOND (X . . . Y) OTHERS (A): 1; 0.026; 0.200
REMARK 3 SYMMETRY VDW REFINED ATOMS (A): 23; 0.705;
0.200
REMARK 3 SYMMETRY VDW OTHERS (A): 86; 0.356; 0.200
REMARK 3 SYMMETRY H-BOND REFINED ATOMS (A): 16; 0.630;
0.200
REMARK 3
REMARK 3 ISOTROPIC THERMAL FACTOR RESTRAINTS. COUNT
RMS WEIGHT
REMARK 3 MAIN-CHAIN BOND REFINED ATOMS (A**2): 1080;
1.274; 1.500
REMARK 3 MAIN-CHAIN BOND OTHER ATOMS (A**2): 407;
0.336; 1.500
REMARK 3 MAIN-CHAIN ANGLE REFINED ATOMS (A**2): 1609;
1.704; 2.000
REMARK 3 SIDE-CHAIN BOND REFINED ATOMS (A**2): 750;
2.842; 3.000
REMARK 3 SIDE-CHAIN ANGLE REFINED ATOMS (A**2): 672;
4.040; 4.500;
REMARK 3
REMARK 3 NCS RESTRAINTS STATISTICS
REMARK 3 NUMBER OF NCS GROUPS: NULL
REMARK 3
REMARK 3
REMARK 3 TLS DETAILS
REMARK 3 NUMBER OF TLS GROUPS: 2
REMARK 3 ATOM RECORD CONTAINS RESIDUAL B FACTORS ONLY
REMARK 3
REMARK 3 TLS GROUP: 1
REMARK 3 NUMBER OF COMPONENTS GROUP: 1
REMARK 3 COMPONENTS C SSSEQI TO C SSSEQI
REMARK 3 RESIDUE RANGE: A 12 A 196
REMARK 3 ORIGIN FOR THE GROUP (A): 24.6062 11.8279
51.1982
REMARK 3 T TENSOR
REMARK 3 T11: −0.0492 T22: −0.0141
REMARK 3 T33: −0.0334 T12: −0.0003
REMARK 3 T13: 0.0030 T23: −0.0021
REMARK 3 L TENSOR
REMARK 3 L11: 1.2428 L22: 0.2699
REMARK 3 L33: 0.4593 L12: −0.0754
REMARK 3 L13: −0.3122 L23: 0.0783
REMARK 3 S TENSOR
REMARK 3 S11: 0.0019 S12: 0.0337 S13: −0.0331
REMARK 3 S21: −0.0179 S22: −0.0002 S23: −0.0058
REMARK 3 S31: −0.0101 S32: −0.325 S33: −0.0018
REMARK 3
REMARK 3 TLS GROUP: 2
REMARK 3 NUMBER OF COMPONENTS GROUP: 1
REMARK 3 COMPONENTS C SSSEQI TO C SSSEQI
REMARK 3 RESIDUE RANGE: P 251 P 264
REMARK 3 ORIGIN FOR THE GROUP (A): 31.4082 0.9166
39.8199
REMARK 3 T TENSOR
REMARK 3 T11: −0.439 T22: −0.0267
REMARK 3 T33: −0.0328 T12: 0.0096
REMARK 3 T13: 0.0511 T23: −0.0513
REMARK 3 L TENSOR
REMARK 3 L11: 5.1441 L22: 2.1674
REMARK 3 L33: 7.0541 L12: 1.1502
REMARK 3 L13: 3.4336 L23: −0.5897
REMARK 3 S TENSOR
REMARK 3 S11: −0.1929 S12: 0.1435 S13: −0.2481
REMARK 3 S21: −0.1624 S22: 0.0877 S23: −0.1382
REMARK 3 S31: 0.2811 S32: 0.1920 S33: 0.1053
REMARK 3
REMARK 3
REMARK 3 BULK SOLVENT MODELLING.
REMARK 3 METHOD USED: MASK
REMARK 3 PARAMETERS FOR MASK CALCUALTION
REMARK 3 VDW PROBE RADIUS: 1.20
REMARK 3 ION PROBE RADIUS: 0.80
REMARK 3 SHRINKAGE RADIUS: 0.80
REMARK 3
REMARK 3 OTHER REFINEMENT REMARKS:
REMARK 3 HYDROGENS HAVE BEEN ADDED IN THE RIDING
POSITIONS
REMARK 3
SSBOND 1 CYS A 61 CYS A 184
SSBOND 2 CYS A 97 CYS A 107
CISPEP 1 PHE A 35 PRO A 36 0.00
CISPEP 2 THR A 127 PRO A 128 0.00
CISPEP 3 ASN A 133 PRO A 134 0.00
CISPEP 4 GLY A 167 PRO A 168 0.00
CRYST1 60.972 60.972 151.681 90.00 90.00 90.00 P 41 21
2
SCALE1 0.016401 0.000000 0.000000 0.00000
SCALE2 0.000000 0.016401 0.000000 0.00000
SCALE3 0.000000 0.000000 0.006593 0.00000

| | | | | | | | | | | | |
|---|---|---|---|---|---|---|---|---|---|---|---|
| ATOM | 1 | N | HIS | A | 12 | 33.704 | 21.776 | 75.467 | 1.00 | 23.03 | N |
| ATOM | 2 | CA | HIS | A | 12 | 34.487 | 22.989 | 75.312 | 1.00 | 20.91 | C |
| ATOM | 4 | CB | HIS | A | 12 | 33.719 | 24.090 | 74.745 | 1.00 | 20.12 | C |
| ATOM | 7 | CG | HIS | A | 12 | 34.117 | 24.583 | 73.396 | 1.00 | 2.00 | C |
| ATOM | 8 | ND1 | HIS | A | 12 | 33.142 | 24.401 | 72.432 | 1.00 | 18.31 | N |
| ATOM | 10 | CE1 | HIS | A | 12 | 33.487 | 25.097 | 71.361 | 1.00 | 22.05 | C |
| ATOM | 12 | NE2 | HIS | A | 12 | 34.473 | 25.876 | 71.733 | 1.00 | 17.59 | N |
| ATOM | 14 | CD2 | HIS | A | 12 | 34.710 | 25.639 | 73.059 | 1.00 | 2.00 | C |
| ATOM | 16 | C | HIS | A | 12 | 34.821 | 23.565 | 76.638 | 1.00 | 23.03 | C |
| ATOM | 17 | O | HIS | A | 12 | 35.059 | 24.763 | 76.648 | 1.00 | 26.11 | O |
| ATOM | 21 | N | HIS | A | 13 | 34.796 | 22.824 | 77.760 | 1.00 | 24.22 | N |
| ATOM | 22 | CA | HIS | A | 13 | 35.792 | 23.137 | 78.752 | 1.00 | 21.79 | C |
| ATOM | 24 | CB | HIS | A | 13 | 35.635 | 22.485 | 80.143 | 1.00 | 24.26 | C |
| ATOM | 27 | CG | HIS | A | 13 | 36.377 | 21.203 | 80.304 | 1.00 | 27.78 | C |
| ATOM | 28 | ND1 | HIS | A | 13 | 35.792 | 19.975 | 80.068 | 1.00 | 33.44 | N |
| ATOM | 30 | CE1 | HIS | A | 13 | 36.686 | 19.023 | 80.270 | 1.00 | 32.20 | C |
| ATOM | 32 | NE2 | HIS | A | 13 | 37.833 | 19.593 | 80.595 | 1.00 | 31.56 | N |
| ATOM | 34 | CD2 | HIS | A | 13 | 37.666 | 20.956 | 80.618 | 1.00 | 28.51 | C |
| ATOM | 36 | C | HIS | A | 13 | 37.116 | 22.806 | 78.029 | 1.00 | 19.23 | C |
| ATOM | 37 | O | HIS | A | 13 | 38.060 | 23.526 | 78.235 | 1.00 | 18.18 | O |
| ATOM | 39 | N | HIS | A | 14 | 37.153 | 21.811 | 77.114 | 1.00 | 16.79 | N |
| ATOM | 40 | CA | HIS | A | 14 | 38.201 | 21.799 | 76.068 | 1.00 | 16.44 | C |
| ATOM | 42 | CB | HIS | A | 14 | 39.054 | 20.546 | 76.115 | 1.00 | 16.82 | C |
| ATOM | 45 | CG | HIS | A | 14 | 39.769 | 20.380 | 77.409 | 1.00 | 18.04 | C |
| ATOM | 46 | ND1 | HIS | A | 14 | 39.709 | 19.220 | 78.150 | 1.00 | 22.52 | N |
| ATOM | 48 | CE1 | HIS | A | 14 | 40.403 | 19.384 | 79.261 | 1.00 | 20.48 | C |
| ATOM | 50 | NE2 | HIS | A | 14 | 40.900 | 20.609 | 79.272 | 1.00 | 22.11 | N |
| ATOM | 52 | CD2 | HIS | A | 14 | 40.530 | 21.245 | 78.113 | 1.00 | 19.51 | C |
| ATOM | 54 | C | HIS | A | 14 | 37.569 | 21.900 | 74.708 | 1.00 | 13.95 | C |
| ATOM | 55 | O | HIS | A | 14 | 36.698 | 21.131 | 74.396 | 1.00 | 13.43 | O |
| ATOM | 57 | N | HIS | A | 15 | 38.008 | 22.870 | 73.924 | 1.00 | 11.83 | N |
| ATOM | 58 | CA | HIS | A | 15 | 37.403 | 23.182 | 72.623 | 1.00 | 11.43 | C |
| ATOM | 60 | CB | HIS | A | 15 | 37.811 | 24.582 | 72.208 | 1.00 | 12.51 | C |
| ATOM | 63 | CG | HIS | A | 15 | 37.613 | 25.585 | 73.298 | 1.00 | 15.38 | C |
| ATOM | 64 | ND1 | HIS | A | 15 | 36.373 | 26.065 | 73.622 | 1.00 | 18.20 | N |
| ATOM | 66 | CE1 | HIS | A | 15 | 36.475 | 26.923 | 74.627 | 1.00 | 15.56 | C |
| ATOM | 68 | NE2 | HIS | A | 15 | 37.744 | 26.993 | 74.977 | 1.00 | 16.84 | N |
| ATOM | 70 | CD2 | HIS | A | 15 | 38.471 | 26.148 | 74.173 | 1.00 | 14.49 | C |
| ATOM | 72 | C | HIS | A | 15 | 37.823 | 22.178 | 71.572 | 1.00 | 10.00 | C |
| ATOM | 73 | O | HIS | A | 15 | 39.029 | 21.913 | 71.367 | 1.00 | 9.89 | O |
| ATOM | 75 | N | HIS | A | 16 | 36.829 | 21.605 | 70.889 | 1.00 | 9.16 | N |
| ATOM | 76 | CA | HIS | A | 16 | 37.126 | 20.594 | 69.895 | 1.00 | 8.66 | C |
| ATOM | 78 | CB | HIS | A | 16 | 37.485 | 19.278 | 70.572 | 1.00 | 9.36 | C |
| ATOM | 81 | CG | HIS | A | 16 | 36.378 | 18.666 | 71.384 | 1.00 | 12.06 | C |
| ATOM | 82 | ND1 | HIS | A | 16 | 36.052 | 19.115 | 72.645 | 1.00 | 13.77 | N |
| ATOM | 84 | CE1 | HIS | A | 16 | 35.077 | 18.366 | 73.134 | 1.00 | 16.21 | C |
| ATOM | 86 | NE2 | HIS | A | 16 | 34.785 | 17.427 | 72.245 | 1.00 | 14.78 | N |
| ATOM | 88 | CD2 | HIS | A | 16 | 35.606 | 17.579 | 71.155 | 1.00 | 13.75 | C |
| ATOM | 90 | C | HIS | A | 16 | 35.957 | 20.379 | 68.944 | 1.00 | 8.83 | C |
| ATOM | 91 | O | HIS | A | 16 | 34.834 | 20.813 | 69.251 | 1.00 | 9.76 | O |
| ATOM | 93 | N | GLU | A | 17 | 36.189 | 19.627 | 67.866 | 1.00 | 7.24 | N |
| ATOM | 94 | CA | GLU | A | 17 | 35.066 | 19.171 | 67.022 | 1.00 | 7.03 | C |
| ATOM | 96 | CB | GLU | A | 17 | 35.446 | 19.014 | 65.549 | 1.00 | 7.82 | C |
| ATOM | 99 | CG | GLU | A | 17 | 35.649 | 20.369 | 64.832 | 1.00 | 6.61 | C |
| ATOM | 102 | CD | GLU | A | 17 | 36.201 | 20.232 | 63.446 | 1.00 | 8.29 | C |
| ATOM | 103 | OE1 | GLU | A | 17 | 36.658 | 19.131 | 63.080 | 1.00 | 8.96 | O |
| ATOM | 104 | OE2 | GLU | A | 17 | 36.147 | 21.259 | 62.691 | 1.00 | 8.78 | O |
| ATOM | 105 | C | GLU | A | 17 | 34.638 | 17.804 | 67.554 | 1.00 | 7.32 | C |
| ATOM | 106 | O | GLU | A | 17 | 35.474 | 16.991 | 67.936 | 1.00 | 8.43 | O |
| ATOM | 108 | N | GLU | A | 18 | 33.330 | 17.584 | 67.549 | 1.00 | 7.30 | N |
| ATOM | 109 | CA | GLU | A | 18 | 32.706 | 16.372 | 68.110 | 1.00 | 9.43 | C |
| ATOM | 111 | CB | GLU | A | 18 | 31.807 | 16.800 | 69.239 | 1.00 | 11.29 | C |
| ATOM | 114 | CG | GLU | A | 18 | 31.129 | 15.663 | 69.960 | 1.00 | 13.90 | C |
| ATOM | 117 | CD | GLU | A | 18 | 32.079 | 14.901 | 70.885 | 1.00 | 22.21 | C |
| ATOM | 118 | OE1 | GLU | A | 18 | 33.301 | 15.167 | 70.928 | 1.00 | 22.48 | O |
| ATOM | 119 | OE2 | GLU | A | 18 | 31.574 | 13.976 | 71.533 | 1.00 | 24.63 | O |
| ATOM | 120 | C | GLU | A | 18 | 31.873 | 15.714 | 66.996 | 1.00 | 8.52 | C |
| ATOM | 121 | O | GLU | A | 18 | 31.031 | 16.361 | 66.335 | 1.00 | 7.95 | O |
| ATOM | 123 | N | THR | A | 19 | 32.122 | 14.426 | 66.790 | 1.00 | 7.26 | N |
| ATOM | 124 | CA | THR | A | 19 | 31.423 | 13.651 | 65.774 | 1.00 | 8.12 | C |
| ATOM | 126 | CB | THR | A | 19 | 32.216 | 12.399 | 65.428 | 1.00 | 7.71 | C |
| ATOM | 128 | OG1 | THR | A | 19 | 33.491 | 12.739 | 64.803 | 1.00 | 8.16 | O |
| ATOM | 130 | CG2 | THR | A | 19 | 31.450 | 11.491 | 64.534 | 1.00 | 7.98 | C |
| ATOM | 134 | C | THR | A | 19 | 29.990 | 13.249 | 66.195 | 1.00 | 6.76 | C |
| ATOM | 135 | O | THR | A | 19 | 29.800 | 12.787 | 67.352 | 1.00 | 8.39 | O |
| ATOM | 137 | N | LEU | A | 20 | 29.037 | 13.430 | 65.273 | 1.00 | 7.50 | N |
| ATOM | 138 | CA | LEU | A | 20 | 27.680 | 12.910 | 65.412 | 1.00 | 8.69 | C |
| ATOM | 140 | CB | LEU | A | 20 | 26.663 | 13.989 | 65.076 | 1.00 | 7.93 | C |
| ATOM | 143 | CG | LEU | A | 20 | 26.630 | 15.185 | 66.010 | 1.00 | 8.84 | C |
| ATOM | 145 | CD1 | LEU | A | 20 | 25.765 | 16.318 | 65.438 | 1.00 | 11.10 | C |
| ATOM | 149 | CD2 | LEU | A | 20 | 26.188 | 14.811 | 67.481 | 1.00 | 9.30 | C |
| ATOM | 153 | C | LEU | A | 20 | 27.444 | 11.629 | 64.566 | 1.00 | 9.13 | C |
| ATOM | 154 | O | LEU | A | 20 | 26.607 | 10.792 | 64.953 | 1.00 | 10.80 | O |
| ATOM | 156 | N | LEU | A | 21 | 28.130 | 11.502 | 63.445 | 1.00 | 8.78 | N |
| ATOM | 157 | CA | LEU | A | 21 | 28.000 | 10.372 | 62.568 | 1.00 | 8.95 | C |
| ATOM | 159 | CB | LEU | A | 21 | 26.719 | 10.483 | 61.749 | 1.00 | 8.65 | C |
| ATOM | 162 | CG | LEU | A | 21 | 26.479 | 9.488 | 60.622 | 1.00 | 11.72 | C |
| ATOM | 164 | CD1 | LEU | A | 21 | 26.560 | 8.089 | 61.045 | 1.00 | 18.08 | C |
| ATOM | 168 | CD2 | LEU | A | 21 | 25.080 | 9.799 | 60.008 | 1.00 | 14.08 | C |
| ATOM | 172 | C | LEU | A | 21 | 29.220 | 10.319 | 61.680 | 1.00 | 8.86 | C |
| ATOM | 173 | O | LEU | A | 21 | 29.599 | 11.317 | 61.146 | 1.00 | 9.99 | O |
| ATOM | 175 | N | ASN | A | 22 | 29.816 | 9.139 | 61.512 | 1.00 | 8.68 | N |
| ATOM | 176 | CA | ASN | A | 22 | 30.886 | 8.943 | 60.530 | 1.00 | 7.18 | C |
| ATOM | 178 | CB | ASN | A | 22 | 32.262 | 9.048 | 61.208 | 1.00 | 7.76 | C |
| ATOM | 181 | CG | ASN | A | 22 | 33.411 | 8.909 | 60.207 | 1.00 | 7.95 | C |
| ATOM | 182 | OD1 | ASN | A | 22 | 33.209 | 8.470 | 59.053 | 1.00 | 9.79 | O |
| ATOM | 183 | ND2 | ASN | A | 22 | 34.620 | 9.287 | 60.638 | 1.00 | 11.49 | N |
| ATOM | 186 | C | ASN | A | 22 | 30.694 | 7.591 | 59.869 | 1.00 | 8.44 | C |
| ATOM | 187 | O | ASN | A | 22 | 30.911 | 6.555 | 60.494 | 1.00 | 8.66 | O |
| ATOM | 189 | N | THR | A | 23 | 30.196 | 7.619 | 58.647 | 1.00 | 8.29 | N |
| ATOM | 190 | CA | THR | A | 23 | 29.883 | 6.375 | 57.972 | 1.00 | 8.29 | C |
| ATOM | 192 | CB | THR | A | 23 | 29.277 | 6.589 | 56.556 | 1.00 | 9.39 | C |
| ATOM | 194 | OG1 | THR | A | 23 | 30.222 | 7.277 | 55.734 | 1.00 | 9.28 | O |
| ATOM | 196 | CG2 | THR | A | 23 | 27.976 | 7.304 | 56.639 | 1.00 | 9.26 | C |
| ATOM | 200 | C | THR | A | 23 | 31.078 | 5.392 | 57.853 | 1.00 | 8.69 | C |
| ATOM | 201 | O | THR | A | 23 | 30.879 | 4.188 | 57.814 | 1.00 | 9.41 | O |
| ATOM | 203 | N | LYS | A | 24 | 32.300 | 5.912 | 57.767 | 1.00 | 8.61 | N |
| ATOM | 204 | CA | LYS | A | 24 | 33.504 | 5.081 | 57.613 | 1.00 | 9.10 | C |
| ATOM | 206 | CB | LYS | A | 24 | 34.728 | 5.936 | 57.267 | 1.00 | 8.84 | C |
| ATOM | 209 | CG | LYS | A | 24 | 34.635 | 6.631 | 55.911 | 1.00 | 10.31 | C |
| ATOM | 212 | CD | LYS | A | 24 | 35.793 | 7.539 | 55.642 | 1.00 | 12.72 | C |
| ATOM | 215 | CE | LYS | A | 24 | 35.586 | 8.923 | 56.230 | 1.00 | 19.96 | C |
| ATOM | 218 | NZ | LYS | A | 24 | 36.870 | 9.639 | 56.390 | 1.00 | 21.45 | N |
| ATOM | 222 | C | LYS | A | 24 | 33.767 | 4.202 | 58.849 | 1.00 | 9.30 | C |
| ATOM | 223 | O | LYS | A | 24 | 34.583 | 3.279 | 58.791 | 1.00 | 10.78 | O |
| ATOM | 225 | N | LEU | A | 25 | 33.118 | 4.548 | 59.973 | 1.00 | 7.98 | N |
| ATOM | 226 | CA | LEU | A | 25 | 33.261 | 3.773 | 61.196 | 1.00 | 9.56 | C |
| ATOM | 228 | CB | LEU | A | 25 | 33.431 | 4.698 | 62.394 | 1.00 | 8.59 | C |
| ATOM | 231 | CG | LEU | A | 25 | 34.666 | 5.591 | 62.340 | 1.00 | 11.50 | C |
| ATOM | 233 | CD1 | LEU | A | 25 | 34.608 | 6.552 | 63.511 | 1.00 | 11.62 | C |
| ATOM | 237 | CD2 | LEU | A | 25 | 35.915 | 4.801 | 62.362 | 1.00 | 14.34 | C |
| ATOM | 241 | C | LEU | A | 25 | 32.158 | 2.717 | 61.406 | 1.00 | 9.15 | C |
| ATOM | 242 | O | LEU | A | 25 | 32.218 | 1.955 | 62.379 | 1.00 | 12.15 | O |
| ATOM | 244 | N | GLU | A | 26 | 31.167 | 2.664 | 60.531 | 1.00 | 8.87 | N |
| ATOM | 245 | CA | GLU | A | 26 | 30.142 | 1.641 | 60.585 | 1.00 | 10.20 | C |
| ATOM | 247 | CB | GLU | A | 26 | 29.022 | 1.940 | 59.577 | 1.00 | 10.65 | C |
| ATOM | 250 | CG | GLU | A | 26 | 27.963 | 0.852 | 59.519 | 1.00 | 11.96 | C |
| ATOM | 253 | CD | GLU | A | 26 | 27.288 | 0.604 | 60.839 | 1.00 | 17.08 | C |
| ATOM | 254 | OE1 | GLU | A | 26 | 26.798 | 1.600 | 61.451 | 1.00 | 20.71 | O |
| ATOM | 255 | OE2 | GLU | A | 26 | 27.230 | −0.595 | 61.279 | 1.00 | 16.36 | O |
| ATOM | 256 | C | GLU | A | 26 | 30.749 | 0.284 | 60.259 | 1.00 | 11.21 | C |
| ATOM | 257 | O | GLU | A | 26 | 31.342 | 0.123 | 59.217 | 1.00 | 13.21 | O |
| ATOM | 259 | N | THR | A | 27 | 30.579 | −0.679 | 61.156 | 1.00 | 14.03 | N |
| ATOM | 260 | CA | THR | A | 27 | 31.199 | −1.991 | 60.979 | 1.00 | 16.39 | C |
| ATOM | 262 | CB | THR | A | 27 | 31.779 | −2.520 | 62.316 | 1.00 | 17.17 | C |
| ATOM | 264 | OG1 | THR | A | 27 | 30.725 | −2.729 | 63.251 | 1.00 | 18.71 | O |
| ATOM | 266 | CG2 | THR | A | 27 | 32.793 | −1.537 | 62.898 | 1.00 | 18.99 | C |
| ATOM | 270 | C | THR | A | 27 | 30.254 | −3.045 | 60.378 | 1.00 | 17.58 | C |
| ATOM | 271 | O | THR | A | 27 | 30.728 | −4.107 | 59.928 | 1.00 | 21.24 | O |
| ATOM | 273 | N | ALA | A | 28 | 28.960 | −2.766 | 60.383 | 1.00 | 15.57 | N |
| ATOM | 274 | CA | ALA | A | 28 | 27.977 | −3.671 | 59.830 | 1.00 | 16.89 | C |
| ATOM | 276 | CB | ALA | A | 28 | 26.981 | −4.129 | 60.889 | 1.00 | 16.73 | C |
| ATOM | 280 | C | ALA | A | 28 | 27.369 | −2.859 | 58.721 | 1.00 | 17.49 | C |
| ATOM | 281 | O | ALA | A | 28 | 28.145 | −2.160 | 58.022 | 1.00 | 20.58 | O |
| ATOM | 283 | N | ASP | A | 29 | 26.063 | −2.917 | 58.523 | 1.00 | 15.97 | N |
| ATOM | 284 | CA | ASP | A | 29 | 25.446 | −2.197 | 57.420 | 1.00 | 17.34 | C |
| ATOM | 286 | CB | ASP | A | 29 | 24.522 | −3.136 | 56.640 | 1.00 | 19.00 | C |
| ATOM | 289 | CG | ASP | A | 29 | 25.301 | −4.257 | 55.860 | 1.00 | 22.70 | C |
| ATOM | 290 | OD1 | ASP | A | 29 | 26.556 | −4.303 | 55.845 | 1.00 | 25.94 | O |
| ATOM | 291 | OD2 | ASP | A | 29 | 24.626 | −5.102 | 55.234 | 1.00 | 33.57 | O |
| ATOM | 292 | C | ASP | A | 29 | 24.739 | −0.927 | 57.917 | 1.00 | 15.71 | C |
| ATOM | 293 | O | ASP | A | 29 | 24.199 | −0.841 | 59.071 | 1.00 | 16.45 | O |
| ATOM | 295 | N | LEU | A | 30 | 24.729 | 0.099 | 57.067 | 1.00 | 13.49 | N |
| ATOM | 296 | CA | LEU | A | 30 | 24.132 | 1.369 | 57.449 | 1.00 | 13.28 | C |
| ATOM | 298 | CB | LEU | A | 30 | 24.507 | 2.446 | 56.436 | 1.00 | 12.20 | C |
| ATOM | 301 | CG | LEU | A | 30 | 25.968 | 2.938 | 56.482 | 1.00 | 11.60 | C |
| ATOM | 303 | CD1 | LEU | A | 30 | 26.311 | 3.826 | 55.253 | 1.00 | 10.66 | C |
| ATOM | 307 | CD2 | LEU | A | 30 | 26.230 | 3.709 | 57.775 | 1.00 | 13.79 | C |
| ATOM | 311 | C | LEU | A | 30 | 22.606 | 1.398 | 57.639 | 1.00 | 15.47 | C |
| ATOM | 312 | O | LEU | A | 30 | 22.095 | 2.178 | 58.420 | 1.00 | 19.16 | O |
| ATOM | 314 | N | LYS | A | 31 | 21.916 | 0.608 | 56.880 | 1.00 | 16.56 | N |
| ATOM | 315 | CA | LYS | A | 31 | 20.435 | 0.542 | 57.010 | 1.00 | 16.90 | C |
| ATOM | 317 | CB | LYS | A | 31 | 19.967 | 0.052 | 58.412 | 1.00 | 17.83 | C |
| ATOM | 324 | C | LYS | A | 31 | 19.707 | 1.845 | 56.679 | 1.00 | 16.92 | C |
| ATOM | 325 | O | LYS | A | 31 | 18.606 | 2.161 | 57.233 | 1.00 | 18.10 | O |
| ATOM | 327 | N | TRP | A | 32 | 20.280 | 2.654 | 55.794 | 1.00 | 14.03 | N |
| ATOM | 328 | CA | TRP | A | 32 | 19.619 | 3.894 | 55.463 | 1.00 | 12.43 | C |
| ATOM | 330 | CB | TRP | A | 32 | 20.553 | 4.837 | 54.691 | 1.00 | 11.17 | C |
| ATOM | 333 | CG | TRP | A | 32 | 21.612 | 5.473 | 55.539 | 1.00 | 10.85 | C |
| ATOM | 334 | CD1 | TRP | A | 32 | 21.845 | 5.269 | 56.865 | 1.00 | 11.79 | C |
| ATOM | 336 | NE1 | TRP | A | 32 | 22.923 | 6.044 | 57.280 | 1.00 | 12.05 | N |
| ATOM | 338 | CE2 | TRP | A | 32 | 23.415 | 6.724 | 56.214 | 1.00 | 11.63 | C |
| ATOM | 339 | CD2 | TRP | A | 32 | 22.618 | 6.389 | 55.092 | 1.00 | 10.21 | C |
| ATOM | 340 | CE3 | TRP | A | 32 | 22.904 | 6.982 | 53.869 | 1.00 | 10.29 | C |
| ATOM | 342 | CZ3 | TRP | A | 32 | 23.969 | 7.858 | 53.782 | 1.00 | 12.53 | C |
| ATOM | 344 | CH2 | TRP | A | 32 | 24.726 | 8.154 | 54.907 | 1.00 | 11.69 | C |
| ATOM | 346 | CZ2 | TRP | A | 32 | 24.448 | 7.626 | 56.126 | 1.00 | 10.84 | C |
| ATOM | 348 | C | TRP | A | 32 | 18.371 | 3.523 | 54.650 | 1.00 | 11.50 | C |
| ATOM | 349 | O | TRP | A | 32 | 18.311 | 2.522 | 53.944 | 1.00 | 12.52 | O |
| ATOM | 351 | N | VAL | A | 33 | 17.375 | 4.377 | 54.719 | 1.00 | 11.31 | N |
| ATOM | 352 | CA | VAL | A | 33 | 16.065 | 4.099 | 54.102 | 1.00 | 10.50 | C |
| ATOM | 354 | CB | VAL | A | 33 | 14.960 | 4.610 | 55.034 | 1.00 | 10.68 | C |
| ATOM | 356 | CG1 | VAL | A | 33 | 13.594 | 4.610 | 54.343 | 1.00 | 10.00 | C |
| ATOM | 360 | CG2 | VAL | A | 33 | 14.951 | 3.783 | 56.320 | 1.00 | 11.80 | C |
| ATOM | 364 | C | VAL | A | 33 | 16.043 | 4.779 | 52.728 | 1.00 | 10.58 | C |
| ATOM | 365 | O | VAL | A | 33 | 16.485 | 5.929 | 52.557 | 1.00 | 11.61 | O |
| ATOM | 367 | N | THR | A | 34 | 15.540 | 4.048 | 51.738 | 1.00 | 9.11 | N |
| ATOM | 368 | CA | THR | A | 34 | 15.419 | 4.579 | 50.386 | 1.00 | 9.33 | C |
| ATOM | 370 | CB | THR | A | 34 | 16.195 | 3.711 | 49.392 | 1.00 | 10.58 | C |
| ATOM | 372 | OG1 | THR | A | 34 | 15.703 | 2.367 | 49.443 | 1.00 | 12.23 | O |
| ATOM | 374 | CG2 | THR | A | 34 | 17.684 | 3.722 | 49.746 | 1.00 | 11.70 | C |
| ATOM | 378 | C | THR | A | 34 | 13.937 | 4.638 | 49.958 | 1.00 | 9.28 | C |
| ATOM | 379 | O | THR | A | 34 | 13.072 | 3.984 | 50.540 | 1.00 | 10.10 | O |
| ATOM | 381 | N | PHE | A | 35 | 13.676 | 5.457 | 48.934 | 1.00 | 8.75 | N |
| ATOM | 382 | CA | PHE | A | 35 | 12.338 | 5.626 | 48.370 | 1.00 | 8.92 | C |
| ATOM | 384 | CB | PHE | A | 35 | 11.574 | 6.664 | 49.204 | 1.00 | 9.31 | C |
| ATOM | 387 | CG | PHE | A | 35 | 10.237 | 7.021 | 48.650 | 1.00 | 10.11 | C |
| ATOM | 388 | CD1 | PHE | A | 35 | 9.107 | 6.312 | 49.017 | 1.00 | 10.65 | C |
| ATOM | 390 | CE1 | PHE | A | 35 | 7.868 | 6.655 | 48.510 | 1.00 | 12.03 | C |
| ATOM | 392 | CZ | PHE | A | 35 | 7.761 | 7.700 | 47.584 | 1.00 | 10.81 | C |
| ATOM | 394 | CE2 | PHE | A | 35 | 8.859 | 8.421 | 47.249 | 1.00 | 11.68 | C |
| ATOM | 396 | CD2 | PHE | A | 35 | 10.098 | 8.088 | 47.764 | 1.00 | 10.45 | C |
| ATOM | 398 | C | PHE | A | 35 | 12.483 | 6.122 | 46.940 | 1.00 | 9.83 | C |
| ATOM | 399 | O | PHE | A | 35 | 13.330 | 6.968 | 46.666 | 1.00 | 10.00 | O |
| ATOM | 401 | N | PRO | A | 36 | 11.600 | 5.685 | 46.025 | 1.00 | 9.75 | N |
| ATOM | 402 | CA | PRO | A | 36 | 10.481 | 4.741 | 46.162 | 1.00 | 10.89 | C |
| ATOM | 404 | CB | PRO | A | 36 | 9.557 | 5.200 | 45.048 | 1.00 | 10.66 | C |
| ATOM | 407 | CG | PRO | A | 36 | 10.437 | 5.623 | 44.008 | 1.00 | 10.68 | C |
| ATOM | 410 | CD | PRO | A | 36 | 11.597 | 6.320 | 44.697 | 1.00 | 10.68 | C |
| ATOM | 413 | C | PRO | A | 36 | 10.812 | 3.244 | 45.986 | 1.00 | 10.85 | C |
| ATOM | 414 | O | PRO | A | 36 | 9.860 | 2.383 | 45.907 | 1.00 | 12.56 | O |
| ATOM | 415 | N | GLN | A | 37 | 12.093 | 2.887 | 45.961 | 1.00 | 11.74 | N |
| ATOM | 416 | CA | GLN | A | 37 | 12.490 | 1.460 | 45.869 | 1.00 | 12.44 | C |
| ATOM | 418 | CB | GLN | A | 37 | 12.088 | 0.679 | 47.099 | 1.00 | 12.50 | C |
| ATOM | 421 | CG | GLN | A | 37 | 12.730 | 1.215 | 48.358 | 1.00 | 14.22 | C |
| ATOM | 424 | CD | GLN | A | 37 | 12.498 | 0.370 | 49.582 | 1.00 | 16.16 | C |
| ATOM | 425 | OE1 | GLN | A | 37 | 11.862 | −0.674 | 49.520 | 1.00 | 21.49 | O |
| ATOM | 426 | NE2 | GLN | A | 37 | 12.953 | 0.843 | 50.710 | 1.00 | 22.36 | N |
| ATOM | 429 | C | GLN | A | 37 | 12.015 | 0.793 | 44.586 | 1.00 | 11.98 | C |
| ATOM | 430 | O | GLN | A | 37 | 11.424 | −0.304 | 44.594 | 1.00 | 15.13 | O |
| ATOM | 432 | N | VAL | A | 38 | 12.253 | 1.474 | 43.488 | 1.00 | 12.06 | N |
| ATOM | 433 | CA | VAL | A | 38 | 11.939 | 0.961 | 42.159 | 1.00 | 12.33 | C |
| ATOM | 435 | CB | VAL | A | 38 | 11.084 | 1.982 | 41.362 | 1.00 | 12.47 | C |
| ATOM | 437 | CG1 | VAL | A | 38 | 9.796 | 2.290 | 42.117 | 1.00 | 13.77 | C |
| ATOM | 441 | CG2 | VAL | A | 38 | 11.830 | 3.262 | 41.037 | 1.00 | 13.76 | C |
| ATOM | 445 | C | VAL | A | 38 | 13.248 | 0.578 | 41.452 | 1.00 | 13.01 | C |
| ATOM | 446 | O | VAL | A | 38 | 14.332 | 0.723 | 42.011 | 1.00 | 12.39 | O |
| ATOM | 448 | N | ASP | A | 39 | 13.131 | 0.043 | 40.242 | 1.00 | 13.10 | N |
| ATOM | 449 | CA | ASP | A | 39 | 14.318 | −0.237 | 39.441 | 1.00 | 14.56 | C |
| ATOM | 451 | CB | ASP | A | 39 | 13.911 | −0.652 | 38.020 | 1.00 | 15.71 | C |
| ATOM | 454 | CG | ASP | A | 39 | 13.239 | −2.028 | 37.949 | 1.00 | 20.14 | C |
| ATOM | 455 | OD1 | ASP | A | 39 | 13.261 | −2.810 | 38.933 | 1.00 | 27.15 | O |
| ATOM | 456 | OD2 | ASP | A | 39 | 12.740 | −2.346 | 36.852 | 1.00 | 25.39 | O |
| ATOM | 457 | C | ASP | A | 39 | 15.199 | 0.996 | 39.390 | 1.00 | 12.97 | C |
| ATOM | 458 | O | ASP | A | 39 | 14.705 | 2.129 | 39.202 | 1.00 | 13.90 | O |
| ATOM | 460 | N | GLY | A | 40 | 16.503 | 0.789 | 39.588 | 1.00 | 12.14 | N |
| ATOM | 461 | CA | GLY | A | 40 | 17.447 | 1.873 | 39.553 | 1.00 | 11.59 | C |
| ATOM | 464 | C | GLY | A | 40 | 17.750 | 2.479 | 40.907 | 1.00 | 10.89 | C |
| ATOM | 465 | O | GLY | A | 40 | 18.521 | 3.419 | 41.007 | 1.00 | 11.12 | O |
| ATOM | 467 | N | GLN | A | 41 | 17.097 | 1.967 | 41.946 | 1.00 | 11.44 | N |
| ATOM | 468 | CA | GLN | A | 41 | 17.245 | 2.470 | 43.296 | 1.00 | 10.98 | C |
| ATOM | 470 | CB | GLN | A | 41 | 16.397 | 1.644 | 44.282 | 1.00 | 12.13 | C |
| ATOM | 473 | CG | GLN | A | 41 | 16.743 | 0.139 | 44.297 | 1.00 | 12.95 | C |
| ATOM | 476 | CD | GLN | A | 41 | 15.707 | −0.648 | 45.075 | 1.00 | 15.14 | C |
| ATOM | 477 | OE1 | GLN | A | 41 | 15.550 | −0.435 | 46.256 | 1.00 | 18.59 | O |
| ATOM | 478 | NE2 | GLN | A | 41 | 15.003 | −1.543 | 44.411 | 1.00 | 22.18 | N |
| ATOM | 481 | C | GLN | A | 41 | 18.683 | 2.545 | 43.803 | 1.00 | 10.18 | C |
| ATOM | 482 | O | GLN | A | 41 | 19.559 | 1.787 | 43.358 | 1.00 | 10.73 | O |
| ATOM | 484 | N | TRP | A | 42 | 18.913 | 3.449 | 44.758 | 1.00 | 9.29 | N |
| ATOM | 485 | CA | TRP | A | 42 | 20.175 | 3.475 | 45.500 | 1.00 | 8.62 | C |
| ATOM | 487 | CB | TRP | A | 42 | 20.067 | 4.399 | 46.695 | 1.00 | 9.35 | C |
| ATOM | 490 | CG | TRP | A | 42 | 20.068 | 5.871 | 46.455 | 1.00 | 8.24 | C |
| ATOM | 491 | CD1 | TRP | A | 42 | 18.985 | 6.677 | 46.392 | 1.00 | 9.84 | C |
| ATOM | 493 | NE1 | TRP | A | 42 | 19.362 | 7.987 | 46.236 | 1.00 | 9.90 | N |
| ATOM | 495 | CE2 | TRP | A | 42 | 20.736 | 8.048 | 46.247 | 1.00 | 8.26 | C |
| ATOM | 496 | CD2 | TRP | A | 42 | 21.209 | 6.727 | 46.362 | 1.00 | 8.34 | C |
| ATOM | 497 | CE3 | TRP | A | 42 | 22.600 | 6.506 | 46.347 | 1.00 | 9.51 | C |
| ATOM | 499 | CZ3 | TRP | A | 42 | 23.436 | 7.588 | 46.260 | 1.00 | 9.12 | C |
| ATOM | 501 | CH2 | TRP | A | 42 | 22.956 | 8.883 | 46.090 | 1.00 | 11.10 | C |
| ATOM | 503 | CZ2 | TRP | A | 42 | 21.601 | 9.144 | 46.088 | 1.00 | 9.47 | C |
| ATOM | 505 | C | TRP | A | 42 | 20.501 | 2.061 | 46.079 | 1.00 | 9.65 | C |
| ATOM | 506 | O | TRP | A | 42 | 16.585 | 1.348 | 46.537 | 1.00 | 11.45 | O |
| ATOM | 508 | N | GLU | A | 43 | 21.772 | 1.681 | 46.012 | 1.00 | 9.40 | N |
| ATOM | 509 | CA | GLU | A | 43 | 22.284 | 0.387 | 46.512 | 1.00 | 10.45 | C |
| ATOM | 511 | CB | AGLU | A | 43 | 22.746 | −0.490 | 45.364 | 0.50 | 10.06 | C |
| ATOM | 512 | CB | BGLU | A | 43 | 22.784 | −0.537 | 45.338 | 0.50 | 10.16 | C |
| ATOM | 517 | CG | AGLU | A | 43 | 21.634 | −0.910 | 44.427 | 0.50 | 12.10 | C |
| ATOM | 518 | CG | BGLU | A | 43 | 23.036 | −2.043 | 45.750 | 0.50 | 10.29 | C |
| ATOM | 523 | CD | AGLU | A | 43 | 22.142 | −1.894 | 43.380 | 0.50 | 14.51 | C |
| ATOM | 524 | CD | BGLU | A | 43 | 23.688 | −3.052 | 44.695 | 0.50 | 13.19 | C |
| ATOM | 525 | OE1 | AGLU | A | 43 | 23.302 | −2.357 | 43.490 | 0.50 | 20.44 | O |
| ATOM | 526 | OE1 | BGLU | A | 43 | 23.946 | −2.788 | 43.499 | 0.50 | 12.08 | O |
| ATOM | 527 | OE2 | AGLU | A | 43 | 21.367 | −2.226 | 42.455 | 0.50 | 22.84 | O |
| ATOM | 528 | OE2 | BGLU | A | 43 | 23.962 | −4.202 | 45.106 | 0.50 | 15.03 | O |
| ATOM | 529 | C | GLU | A | 43 | 23.411 | 0.602 | 47.474 | 1.00 | 9.78 | C |
| ATOM | 530 | O | GLU | A | 43 | 24.319 | 1.419 | 47.216 | 1.00 | 9.79 | O |
| ATOM | 532 | N | GLU | A | 44 | 23.423 | −0.158 | 48.567 | 1.00 | 8.15 | N |
| ATOM | 533 | CA | GLU | A | 44 | 24.572 | −0.124 | 49.511 | 1.00 | 9.00 | C |
| ATOM | 535 | CB | GLU | A | 44 | 24.095 | −0.475 | 50.912 | 1.00 | 10.13 | C |
| ATOM | 538 | CG | GLU | A | 44 | 25.149 | −0.187 | 51.988 | 1.00 | 10.06 | C |
| ATOM | 541 | CD | GLU | A | 44 | 24.692 | −0.489 | 53.391 | 1.00 | 11.49 | C |
| ATOM | 542 | OE1 | GLU | A | 44 | 23.500 | −0.851 | 53.554 | 1.00 | 12.88 | O |
| ATOM | 543 | OE2 | GLU | A | 44 | 25.529 | −0.343 | 54.332 | 1.00 | 12.69 | O |
| ATOM | 544 | C | GLU | A | 44 | 25.636 | −1.122 | 49.065 | 1.00 | 9.85 | C |
| ATOM | 545 | O | GLU | A | 44 | 25.290 | −2.321 | 48.823 | 1.00 | 10.25 | O |
| ATOM | 547 | N | LEU | A | 45 | 26.879 | −0.659 | 48.967 | 1.00 | 10.70 | N |
| ATOM | 548 | CA | LEU | A | 45 | 28.020 | −1.454 | 48.498 | 1.00 | 12.02 | C |
| ATOM | 550 | CB | LEU | A | 45 | 28.459 | −0.992 | 47.102 | 1.00 | 12.79 | C |
| ATOM | 553 | CG | LEU | A | 45 | 27.360 | −1.452 | 46.135 | 1.00 | 15.99 | C |
| ATOM | 555 | CD1 | LEU | A | 45 | 26.815 | −0.440 | 45.202 | 1.00 | 20.04 | C |
| ATOM | 559 | CD2 | LEU | A | 45 | 27.663 | −2.822 | 45.541 | 1.00 | 15.67 | C |
| ATOM | 563 | C | LEU | A | 45 | 29.217 | −1.302 | 49.411 | 1.00 | 12.37 | C |
| ATOM | 564 | O | LEU | A | 45 | 29.462 | −0.213 | 49.957 | 1.00 | 12.81 | O |
| ATOM | 566 | N | ASER | A | 46 | 29.991 | −2.366 | 49.589 | 0.50 | 13.04 | N |
| ATOM | 567 | N | BSER | A | 46 | 29.981 | −2.386 | 49.551 | 0.50 | 12.82 | N |
| ATOM | 568 | CA | ASER | A | 46 | 31.244 | −2.192 | 50.328 | 0.50 | 13.68 | C |
| ATOM | 569 | CA | BSER | A | 46 | 31.300 | −2.357 | 50.215 | 0.50 | 13.35 | C |
| ATOM | 572 | CB | ASER | A | 46 | 31.713 | −3.484 | 50.957 | 0.50 | 14.34 | C |
| ATOM | 573 | CB | BSER | A | 46 | 31.770 | −3.818 | 50.405 | 0.50 | 13.78 | C |
| ATOM | 578 | OG | ASER | A | 46 | 32.852 | −3.193 | 51.749 | 0.50 | 17.56 | O |
| ATOM | 579 | OG | BSER | A | 46 | 32.829 | −3.971 | 51.338 | 0.50 | 16.91 | O |
| ATOM | 582 | C | ASER | A | 46 | 32.362 | −1.625 | 49.451 | 0.50 | 13.16 | C |
| ATOM | 583 | C | BSER | A | 46 | 32.319 | −1.559 | 49.362 | 0.50 | 12.99 | C |
| ATOM | 584 | O | ASER | A | 46 | 32.724 | −2.219 | 48.426 | 0.50 | 14.44 | O |
| ATOM | 585 | O | BSER | A | 46 | 32.524 | −1.885 | 48.203 | 0.50 | 14.64 | O |
| ATOM | 588 | N | GLY | A | 47 | 32.931 | −0.516 | 49.911 | 1.00 | 12.98 | N |
| ATOM | 589 | CA | GLY | A | 47 | 34.067 | 0.174 | 49.264 | 1.00 | 12.65 | C |
| ATOM | 592 | C | GLY | A | 47 | 35.295 | 0.132 | 50.139 | 1.00 | 12.62 | C |
| ATOM | 593 | O | GLY | A | 47 | 35.322 | −0.585 | 51.123 | 1.00 | 11.81 | O |
| ATOM | 595 | N | LEU | A | 48 | 36.324 | 0.859 | 49.719 | 1.00 | 12.18 | N |
| ATOM | 596 | CA | LEU | A | 48 | 37.609 | 0.936 | 50.440 | 1.00 | 13.20 | C |
| ATOM | 598 | CB | LEU | A | 48 | 38.703 | 0.319 | 49.567 | 1.00 | 14.06 | C |
| ATOM | 601 | CG | LEU | A | 48 | 38.539 | −1.139 | 49.254 | 1.00 | 16.14 | C |
| ATOM | 603 | CD1 | LEU | A | 48 | 39.564 | −1.533 | 48.222 | 1.00 | 17.56 | C |
| ATOM | 607 | CD2 | LEU | A | 48 | 38.722 | −1.970 | 50.503 | 1.00 | 15.62 | C |
| ATOM | 611 | C | LEU | A | 48 | 37.989 | 2.347 | 50.759 | 1.00 | 14.96 | C |
| ATOM | 612 | O | LEU | A | 48 | 37.853 | 3.224 | 49.886 | 1.00 | 17.67 | O |
| ATOM | 614 | N | ASP | A | 49 | 38.457 | 2.587 | 51.982 | 1.00 | 14.25 | N |
| ATOM | 615 | CA | ASP | A | 49 | 38.898 | 3.925 | 52.362 | 1.00 | 15.81 | C |
| ATOM | 617 | CB | ASP | A | 49 | 38.508 | 4.250 | 53.792 | 1.00 | 15.75 | C |
| ATOM | 620 | CG | ASP | A | 49 | 39.232 | 3.414 | 54.842 | 1.00 | 16.73 | C |
| ATOM | 621 | OD1 | ASP | A | 49 | 40.250 | 2.750 | 54.552 | 1.00 | 16.95 | O |
| ATOM | 622 | OD2 | ASP | A | 49 | 38.742 | 3.421 | 56.007 | 1.00 | 23.27 | O |
| ATOM | 623 | C | ASP | A | 49 | 40.407 | 4.086 | 52.098 | 1.00 | 15.71 | C |
| ATOM | 624 | O | ASP | A | 49 | 40.991 | 3.232 | 51.452 | 1.00 | 15.44 | O |
| ATOM | 626 | N | GLU | A | 50 | 40.984 | 5.201 | 52.574 | 1.00 | 16.80 | N |
| ATOM | 627 | CA | GLU | A | 50 | 42.447 | 5.505 | 52.380 | 1.00 | 16.83 | C |
| ATOM | 629 | CB | GLU | A | 50 | 42.754 | 6.946 | 52.825 | 1.00 | 16.31 | C |
| ATOM | 632 | CG | GLU | A | 50 | 42.096 | 7.950 | 52.002 | 1.00 | 17.42 | C |
| ATOM | 635 | CD | GLU | A | 50 | 42.520 | 9.388 | 52.332 | 1.00 | 15.16 | C |
| ATOM | 636 | OE1 | GLU | A | 50 | 43.455 | 9.619 | 53.192 | 1.00 | 14.33 | O |
| ATOM | 637 | OE2 | GLU | A | 50 | 41.849 | 10.255 | 51.735 | 1.00 | 15.53 | O |
| ATOM | 638 | C | GLU | A | 50 | 43.427 | 4.558 | 53.046 | 1.00 | 19.00 | C |
| ATOM | 639 | O | GLU | A | 50 | 44.650 | 4.553 | 52.703 | 1.00 | 19.95 | O |
| ATOM | 641 | N | GLU | A | 51 | 42.934 | 3.766 | 53.982 | 1.00 | 19.32 | N |
| ATOM | 642 | CA | GLU | A | 51 | 43.741 | 2.769 | 54.672 | 1.00 | 20.38 | C |
| ATOM | 644 | CB | GLU | A | 51 | 43.417 | 2.808 | 56.159 | 1.00 | 21.41 | C |
| ATOM | 647 | CG | GLU | A | 51 | 43.711 | 4.160 | 56.784 | 1.00 | 23.95 | C |
| ATOM | 650 | CD | GLU | A | 51 | 43.500 | 4.152 | 58.257 | 1.00 | 24.68 | C |
| ATOM | 651 | OE1 | GLU | A | 51 | 44.373 | 3.593 | 58.950 | 1.00 | 30.31 | O |
| ATOM | 652 | OE2 | GLU | A | 51 | 42.488 | 4.733 | 58.706 | 1.00 | 32.21 | O |
| ATOM | 653 | C | GLU | A | 51 | 43.466 | 1.384 | 54.124 | 1.00 | 19.92 | C |
| ATOM | 654 | O | GLU | A | 51 | 43.931 | 0.378 | 54.697 | 1.00 | 19.56 | O |
| ATOM | 656 | N | GLN | A | 52 | 42.717 | 1.327 | 53.020 | 1.00 | 18.89 | N |
| ATOM | 657 | CA | GLN | A | 52 | 42.328 | 0.054 | 52.400 | 1.00 | 18.69 | C |
| ATOM | 659 | CB | GLN | A | 52 | 43.549 | −0.779 | 52.000 | 1.00 | 19.90 | C |
| ATOM | 666 | C | GLN | A | 52 | 41.423 | −0.789 | 53.285 | 1.00 | 18.80 | C |
| ATOM | 667 | O | GLN | A | 52 | 41.434 | −2.040 | 53.172 | 1.00 | 19.32 | O |
| ATOM | 669 | N | HIS | A | 53 | 40.663 | −0.138 | 54.166 | 1.00 | 17.19 | N |
| ATOM | 670 | CA | HIS | A | 53 | 39.715 | −0.828 | 55.003 | 1.00 | 15.92 | C |
| ATOM | 672 | CB | HIS | A | 53 | 39.600 | −0.162 | 56.376 | 1.00 | 17.51 | C |
| ATOM | 675 | CG | HIS | A | 53 | 40.864 | −0.204 | 57.199 | 1.00 | 20.93 | C |
| ATOM | 676 | ND1 | HIS | A | 53 | 41.949 | −0.994 | 56.884 | 1.00 | 27.78 | N |
| ATOM | 678 | CE1 | HIS | A | 53 | 42.906 | −0.804 | 57.780 | 1.00 | 26.65 | C |
| ATOM | 680 | NE2 | HIS | A | 53 | 42.474 | 0.073 | 58.669 | 1.00 | 27.47 | N |
| ATOM | 682 | CD2 | HIS | A | 53 | 41.199 | 0.457 | 58.334 | 1.00 | 27.24 | C |
| ATOM | 684 | C | HIS | A | 53 | 38.357 | −0.770 | 54.326 | 1.00 | 14.19 | C |
| ATOM | 685 | O | HIS | A | 53 | 38.030 | 0.211 | 53.651 | 1.00 | 13.34 | O |
| ATOM | 687 | N | SER | A | 54 | 37.560 | −1.810 | 54.539 | 1.00 | 13.06 | N |
| ATOM | 688 | CA | ASER | A | 54 | 36.206 | −1.870 | 53.974 | 0.50 | 12.48 | C |
| ATOM | 689 | CA | BSER | A | 54 | 36.210 | −1.865 | 53.965 | 0.50 | 12.34 | C |
| ATOM | 692 | CB | ASER | A | 54 | 35.624 | −3.262 | 54.156 | 0.50 | 12.71 | C |
| ATOM | 693 | CB | BSER | A | 54 | 35.628 | −3.265 | 54.097 | 0.50 | 12.50 | C |
| ATOM | 698 | OG | ASER | A | 54 | 34.435 | −3.423 | 53.403 | 0.50 | 14.69 | O |
| ATOM | 699 | OG | BSER | A | 54 | 36.326 | −4.201 | 53.280 | 0.50 | 13.56 | O |
| ATOM | 702 | C | SER | A | 54 | 35.297 | −0.859 | 54.655 | 1.00 | 12.10 | C |
| ATOM | 703 | O | SER | A | 54 | 35.269 | −0.787 | 55.896 | 1.00 | 13.90 | O |
| ATOM | 705 | N | VAL | A | 55 | 34.535 | −0.100 | 53.854 | 1.00 | 11.09 | N |
| ATOM | 706 | CA | VAL | A | 55 | 33.566 | 0.891 | 54.371 | 1.00 | 10.54 | C |
| ATOM | 708 | CB | VAL | A | 55 | 34.114 | 2.330 | 54.277 | 1.00 | 10.19 | C |
| ATOM | 710 | CG1 | VAL | A | 55 | 35.310 | 2.461 | 55.167 | 1.00 | 13.09 | C |
| ATOM | 714 | CG2 | VAL | A | 55 | 34.460 | 2.665 | 52.856 | 1.00 | 11.17 | C |
| ATOM | 718 | C | VAL | A | 55 | 32.238 | 0.771 | 53.602 | 1.00 | 10.18 | C |
| ATOM | 719 | O | VAL | A | 55 | 32.214 | 0.333 | 52.462 | 1.00 | 11.24 | O |
| ATOM | 721 | N | ARG | A | 56 | 31.135 | 1.140 | 54.233 | 1.00 | 9.01 | N |
| ATOM | 722 | CA | ARG | A | 56 | 29.826 | 1.113 | 53.595 | 1.00 | 9.91 | C |
| ATOM | 724 | CB | ARG | A | 56 | 28.716 | 1.048 | 54.615 | 1.00 | 10.97 | C |
| ATOM | 727 | CG | ARG | A | 56 | 28.801 | −0.147 | 55.563 | 1.00 | 11.58 | C |
| ATOM | 730 | CD | ARG | A | 56 | 29.104 | −1.447 | 54.865 | 1.00 | 15.21 | C |
| ATOM | 733 | NE | ARG | A | 56 | 28.040 | −1.854 | 53.969 | 1.00 | 17.47 | N |
| ATOM | 735 | CZ | ARG | A | 56 | 28.133 | −2.887 | 53.129 | 1.00 | 20.77 | C |
| ATOM | 736 | NH1 | ARG | A | 56 | 27.092 | −3.224 | 52.358 | 1.00 | 22.08 | N |
| ATOM | 739 | NH2 | ARG | A | 56 | 29.247 | −3.592 | 53.071 | 1.00 | 21.45 | N |
| ATOM | 742 | C | ARG | A | 56 | 29.663 | 2.399 | 52.762 | 1.00 | 8.97 | C |
| ATOM | 743 | O | ARG | A | 56 | 29.967 | 3.514 | 53.222 | 1.00 | 9.94 | O |
| ATOM | 745 | N | THR | A | 57 | 29.169 | 2.213 | 51.535 | 1.00 | 9.12 | N |
| ATOM | 746 | CA | THR | A | 57 | 28.921 | 3.316 | 50.591 | 1.00 | 8.61 | C |
| ATOM | 748 | CB | THR | A | 57 | 29.972 | 3.361 | 49.481 | 1.00 | 9.01 | C |
| ATOM | 750 | OG1 | THR | A | 57 | 29.798 | 2.254 | 48.563 | 1.00 | 9.10 | O |
| ATOM | 752 | CG2 | THR | A | 57 | 31.412 | 3.330 | 50.074 | 1.00 | 9.29 | C |
| ATOM | 756 | C | THR | A | 57 | 27.549 | 3.129 | 49.941 | 1.00 | 9.15 | C |
| ATOM | 757 | O | THR | A | 57 | 26.953 | 2.050 | 50.011 | 1.00 | 8.65 | O |
| ATOM | 759 | N | TYR | A | 58 | 27.067 | 4.170 | 49.276 | 1.00 | 8.82 | N |
| ATOM | 760 | CA | TYR | A | 58 | 25.812 | 4.073 | 48.489 | 1.00 | 9.14 | C |
| ATOM | 762 | CB | TYR | A | 58 | 24.694 | 4.925 | 49.121 | 1.00 | 10.04 | C |
| ATOM | 765 | CG | TYR | A | 58 | 24.006 | 4.219 | 50.253 | 1.00 | 9.55 | C |
| ATOM | 766 | CD1 | TYR | A | 58 | 23.002 | 3.253 | 50.008 | 1.00 | 9.24 | C |
| ATOM | 768 | CE1 | TYR | A | 58 | 22.357 | 2.622 | 51.048 | 1.00 | 9.04 | C |
| ATOM | 770 | CZ | TYR | A | 58 | 22.793 | 2.824 | 52.344 | 1.00 | 10.33 | C |
| ATOM | 771 | OH | TYR | A | 58 | 22.181 | 2.115 | 53.417 | 1.00 | 13.23 | O |
| ATOM | 773 | CE2 | TYR | A | 58 | 23.759 | 3.767 | 52.620 | 1.00 | 9.75 | C |
| ATOM | 775 | CD2 | TYR | A | 58 | 24.382 | 4.456 | 51.562 | 1.00 | 10.04 | C |
| ATOM | 777 | C | TYR | A | 58 | 26.066 | 4.544 | 47.058 | 1.00 | 9.09 | C |
| ATOM | 778 | O | TYR | A | 58 | 26.751 | 5.552 | 46.857 | 1.00 | 9.85 | O |
| ATOM | 780 | N | GLU | A | 59 | 25.509 | 3.823 | 46.085 | 1.00 | 7.72 | N |
| ATOM | 781 | CA | GLU | A | 59 | 25.622 | 4.186 | 44.660 | 1.00 | 8.04 | C |
| ATOM | 783 | CB | GLU | A | 59 | 26.491 | 3.177 | 43.912 | 1.00 | 9.06 | C |
| ATOM | 786 | CG | GLU | A | 59 | 27.910 | 3.032 | 44.412 | 1.00 | 8.60 | C |
| ATOM | 789 | CD | GLU | A | 59 | 28.738 | 2.070 | 43.535 | 1.00 | 9.81 | C |
| ATOM | 790 | OE1 | GLU | A | 59 | 28.170 | 1.590 | 42.538 | 1.00 | 9.82 | O |
| ATOM | 791 | OE2 | GLU | A | 59 | 29.925 | 1.884 | 43.849 | 1.00 | 12.02 | O |
| ATOM | 792 | C | GLU | A | 59 | 24.248 | 4.212 | 44.000 | 1.00 | 9.20 | C |
| ATOM | 793 | O | GLU | A | 59 | 23.337 | 3.488 | 44.368 | 1.00 | 8.68 | O |
| ATOM | 795 | N | VAL | A | 60 | 24.140 | 5.064 | 42.988 | 1.00 | 8.44 | N |
| ATOM | 796 | CA | VAL | A | 60 | 22.956 | 5.146 | 42.127 | 1.00 | 7.85 | C |
| ATOM | 798 | CB | VAL | A | 60 | 21.914 | 6.133 | 42.665 | 1.00 | 8.42 | C |
| ATOM | 800 | CG1 | VAL | A | 60 | 22.382 | 7.596 | 42.623 | 1.00 | 8.14 | C |
| ATOM | 804 | CG2 | VAL | A | 60 | 20.563 | 5.969 | 41.977 | 1.00 | 10.07 | C |
| ATOM | 808 | C | VAL | A | 60 | 23.429 | 5.545 | 40.724 | 1.00 | 8.37 | C |
| ATOM | 809 | O | VAL | A | 60 | 24.353 | 6.371 | 40.582 | 1.00 | 8.58 | O |
| ATOM | 811 | N | CYS | A | 61 | 22.861 | 4.928 | 39.697 | 1.00 | 8.88 | N |
| ATOM | 812 | CA | CYS | A | 61 | 23.260 | 5.245 | 38.330 | 1.00 | 9.45 | C |
| ATOM | 814 | CB | CYS | A | 61 | 24.553 | 4.536 | 37.981 | 1.00 | 9.31 | C |
| ATOM | 817 | SG | CYS | A | 61 | 25.313 | 5.075 | 36.382 | 1.00 | 9.25 | S |
| ATOM | 819 | C | CYS | A | 61 | 22.136 | 4.912 | 37.310 | 1.00 | 10.12 | C |
| ATOM | 820 | O | CYS | A | 61 | 22.342 | 4.133 | 36.386 | 1.00 | 10.79 | O |
| ATOM | 822 | N | ASP | A | 62 | 20.988 | 5.558 | 37.497 | 1.00 | 9.85 | N |
| ATOM | 823 | CA | ASP | A | 62 | 19.793 | 5.376 | 36.660 | 1.00 | 9.28 | C |
| ATOM | 825 | CB | ASP | A | 62 | 18.546 | 5.341 | 37.553 | 1.00 | 10.45 | C |
| ATOM | 828 | CG | ASP | A | 62 | 17.292 | 5.094 | 36.806 | 1.00 | 10.86 | C |
| ATOM | 829 | OD1 | ASP | A | 62 | 17.381 | 4.800 | 35.577 | 1.00 | 12.22 | O |
| ATOM | 830 | OD2 | ASP | A | 62 | 16.194 | 5.144 | 37.448 | 1.00 | 11.23 | O |
| ATOM | 831 | C | ASP | A | 62 | 19.788 | 6.503 | 35.646 | 1.00 | 11.31 | C |
| ATOM | 832 | O | ASP | A | 62 | 19.204 | 7.568 | 35.876 | 1.00 | 12.18 | O |
| ATOM | 834 | N | VAL | A | 63 | 20.458 | 6.261 | 34.514 | 1.00 | 11.89 | N |
| ATOM | 835 | CA | VAL | A | 63 | 20.638 | 7.279 | 33.478 | 1.00 | 13.16 | C |
| ATOM | 837 | CB | VAL | A | 63 | 22.122 | 7.642 | 33.304 | 1.00 | 13.22 | C |
| ATOM | 839 | CG1 | VAL | A | 63 | 22.649 | 8.312 | 34.536 | 1.00 | 12.96 | C |
| ATOM | 843 | CG2 | VAL | A | 63 | 22.963 | 6.406 | 32.886 | 1.00 | 14.83 | C |
| ATOM | 847 | C | VAL | A | 63 | 20.081 | 6.837 | 32.138 | 1.00 | 14.23 | C |
| ATOM | 848 | O | VAL | A | 63 | 19.822 | 7.649 | 31.266 | 1.00 | 16.46 | O |
| ATOM | 850 | N | GLN | A | 64 | 19.887 | 5.542 | 31.985 | 1.00 | 15.84 | N |
| ATOM | 851 | CA | GLN | A | 64 | 19.426 | 4.983 | 30.720 | 1.00 | 18.13 | C |
| ATOM | 853 | CB | GLN | A | 64 | 20.275 | 3.754 | 30.377 | 1.00 | 18.23 | C |
| ATOM | 860 | C | GLN | A | 64 | 17.948 | 4.668 | 30.812 | 1.00 | 19.94 | C |
| ATOM | 861 | O | GLN | A | 64 | 17.161 | 5.116 | 29.956 | 1.00 | 22.90 | O |
| ATOM | 863 | N | ARG | A | 65 | 17.549 | 3.933 | 31.849 | 1.00 | 20.14 | N |
| ATOM | 864 | CA | ARG | A | 65 | 16.182 | 3.488 | 32.024 | 1.00 | 19.89 | C |
| ATOM | 866 | CB | ARG | A | 65 | 16.116 | 2.460 | 33.151 | 1.00 | 21.74 | C |
| ATOM | 875 | C | ARG | A | 65 | 15.242 | 4.657 | 32.342 | 1.00 | 19.41 | C |
| ATOM | 876 | O | ARG | A | 65 | 14.266 | 4.904 | 31.598 | 1.00 | 18.18 | O |
| ATOM | 878 | N | ALA | A | 66 | 15.523 | 5.374 | 33.434 | 1.00 | 17.78 | N |
| ATOM | 879 | CA | ALA | A | 66 | 14.647 | 6.477 | 33.834 | 1.00 | 15.90 | C |
| ATOM | 881 | CB | ALA | A | 66 | 13.568 | 6.005 | 34.787 | 1.00 | 17.23 | C |
| ATOM | 885 | C | ALA | A | 66 | 15.425 | 7.631 | 34.441 | 1.00 | 14.82 | C |
| ATOM | 886 | O | ALA | A | 66 | 15.431 | 7.807 | 35.652 | 1.00 | 12.98 | O |
| ATOM | 888 | N | PRO | A | 67 | 16.086 | 8.439 | 33.602 | 1.00 | 13.82 | N |
| ATOM | 889 | CA | PRO | A | 67 | 16.815 | 9.560 | 34.155 | 1.00 | 14.16 | C |
| ATOM | 891 | CB | PRO | A | 67 | 17.626 | 10.096 | 32.966 | 1.00 | 13.92 | C |
| ATOM | 894 | CG | PRO | A | 67 | 16.887 | 9.608 | 31.762 | 1.00 | 13.64 | C |
| ATOM | 897 | CD | PRO | A | 67 | 16.213 | 8.342 | 32.130 | 1.00 | 13.56 | C |
| ATOM | 900 | C | PRO | A | 67 | 15.933 | 10.643 | 34.787 | 1.00 | 13.32 | C |
| ATOM | 901 | O | PRO | A | 67 | 16.461 | 11.477 | 35.529 | 1.00 | 14.01 | O |
| ATOM | 902 | N | GLY | A | 68 | 14.608 | 10.628 | 34.558 | 1.00 | 12.54 | N |
| ATOM | 903 | CA | GLY | A | 68 | 13.755 | 11.553 | 35.252 | 1.00 | 12.21 | C |
| ATOM | 906 | C | GLY | A | 68 | 13.158 | 10.971 | 36.523 | 1.00 | 11.44 | C |
| ATOM | 907 | O | GLY | A | 68 | 12.387 | 11.656 | 37.202 | 1.00 | 12.78 | O |
| ATOM | 909 | N | GLN | A | 69 | 13.483 | 9.724 | 36.869 | 1.00 | 10.14 | N |
| ATOM | 910 | CA | GLN | A | 69 | 12.998 | 9.159 | 38.153 | 1.00 | 10.41 | C |
| ATOM | 912 | CB | GLN | A | 69 | 13.012 | 7.621 | 38.111 | 1.00 | 10.00 | C |
| ATOM | 915 | CG | GLN | A | 69 | 12.675 | 6.939 | 39.427 | 1.00 | 9.92 | C |
| ATOM | 918 | CD | GLN | A | 69 | 11.232 | 7.122 | 39.827 | 1.00 | 10.80 | C |
| ATOM | 919 | OE1 | GLN | A | 69 | 10.307 | 6.749 | 39.064 | 1.00 | 13.37 | O |
| ATOM | 920 | NE2 | GLN | A | 69 | 11.030 | 7.676 | 41.007 | 1.00 | 9.15 | N |
| ATOM | 923 | C | GLN | A | 69 | 13.846 | 9.645 | 39.331 | 1.00 | 10.33 | C |
| ATOM | 924 | O | GLN | A | 69 | 15.071 | 9.470 | 39.355 | 1.00 | 9.39 | O |
| ATOM | 926 | N | ALA | A | 70 | 13.228 | 10.239 | 40.332 | 1.00 | 9.62 | N |
| ATOM | 927 | CA | ALA | A | 70 | 13.970 | 10.585 | 41.559 | 1.00 | 9.05 | C |
| ATOM | 929 | CB | ALA | A | 70 | 13.269 | 11.657 | 42.362 | 1.00 | 10.32 | C |
| ATOM | 933 | C | ALA | A | 70 | 14.159 | 9.350 | 42.419 | 1.00 | 9.58 | C |
| ATOM | 934 | O | ALA | A | 70 | 13.252 | 8.543 | 42.597 | 1.00 | 11.06 | O |
| ATOM | 936 | N | HIS | A | 71 | 15.345 | 9.286 | 43.028 | 1.00 | 7.99 | N |
| ATOM | 937 | CA | HIS | A | 71 | 15.731 | 8.234 | 43.969 | 1.00 | 9.09 | C |
| ATOM | 939 | CB | HIS | A | 71 | 16.817 | 7.328 | 43.364 | 1.00 | 9.38 | C |
| ATOM | 942 | CG | HIS | A | 71 | 16.411 | 6.620 | 42.113 | 1.00 | 9.04 | C |
| ATOM | 943 | ND1 | HIS | A | 71 | 15.569 | 5.527 | 42.122 | 1.00 | 10.50 | N |
| ATOM | 945 | CE1 | HIS | A | 71 | 15.444 | 5.073 | 40.884 | 1.00 | 9.85 | C |
| ATOM | 947 | NE2 | HIS | A | 71 | 16.158 | 5.834 | 40.073 | 1.00 | 8.23 | N |
| ATOM | 949 | CD2 | HIS | A | 71 | 16.779 | 6.806 | 40.817 | 1.00 | 10.81 | C |
| ATOM | 951 | C | HIS | A | 71 | 16.249 | 8.919 | 45.224 | 1.00 | 8.98 | C |
| ATOM | 952 | O | HIS | A | 71 | 17.191 | 9.718 | 45.170 | 1.00 | 9.27 | O |
| ATOM | 954 | N | TRP | A | 72 | 15.621 | 8.599 | 46.357 | 1.00 | 8.10 | N |
| ATOM | 955 | CA | TRP | A | 72 | 15.913 | 9.199 | 47.655 | 1.00 | 8.30 | C |
| ATOM | 957 | CB | TRP | A | 72 | 14.601 | 9.697 | 48.329 | 1.00 | 8.49 | C |
| ATOM | 960 | CG | TRP | A | 72 | 13.929 | 10.870 | 47.685 | 1.00 | 8.08 | C |
| ATOM | 961 | CD1 | TRP | A | 72 | 13.113 | 10.859 | 46.603 | 1.00 | 9.35 | C |
| ATOM | 963 | NE1 | TRP | A | 72 | 12.671 | 12.153 | 46.316 | 1.00 | 10.16 | N |
| ATOM | 965 | CE2 | TRP | A | 72 | 13.200 | 13.002 | 47.244 | 1.00 | 10.46 | C |
| ATOM | 966 | CD2 | TRP | A | 72 | 14.006 | 12.234 | 48.118 | 1.00 | 8.67 | C |
| ATOM | 967 | CE3 | TRP | A | 72 | 14.663 | 12.862 | 49.162 | 1.00 | 10.76 | C |
| ATOM | 969 | CZ3 | TRP | A | 72 | 14.533 | 14.248 | 49.299 | 1.00 | 9.32 | C |
| ATOM | 971 | CH2 | TRP | A | 72 | 13.704 | 14.953 | 48.451 | 1.00 | 8.41 | C |
| ATOM | 973 | CZ2 | TRP | A | 72 | 13.057 | 14.361 | 47.395 | 1.00 | 10.47 | C |
| ATOM | 975 | C | TRP | A | 72 | 16.587 | 8.259 | 48.621 | 1.00 | 8.56 | C |
| ATOM | 976 | O | TRP | A | 72 | 16.277 | 7.069 | 48.685 | 1.00 | 8.28 | O |
| ATOM | 978 | N | LEU | A | 73 | 17.454 | 8.841 | 49.457 | 1.00 | 8.33 | N |
| ATOM | 979 | CA | LEU | A | 73 | 18.276 | 8.127 | 50.455 | 1.00 | 8.49 | C |
| ATOM | 981 | CB | LEU | A | 73 | 19.702 | 8.013 | 49.922 | 1.00 | 8.82 | C |
| ATOM | 984 | CG | LEU | A | 73 | 20.739 | 7.348 | 50.793 | 1.00 | 8.48 | C |
| ATOM | 986 | CD1 | LEU | A | 73 | 20.455 | 5.895 | 51.007 | 1.00 | 9.79 | C |
| ATOM | 990 | CD2 | LEU | A | 73 | 22.131 | 7.516 | 50.167 | 1.00 | 9.77 | C |
| ATOM | 994 | C | LEU | A | 73 | 18.274 | 8.950 | 51.732 | 1.00 | 8.70 | C |
| ATOM | 995 | O | LEU | A | 73 | 18.618 | 10.119 | 51.712 | 1.00 | 9.55 | O |
| ATOM | 997 | N | ARG | A | 74 | 17.906 | 8.347 | 52.844 | 1.00 | 8.25 | N |
| ATOM | 998 | CA | ARG | A | 74 | 17.804 | 9.028 | 54.143 | 1.00 | 8.92 | C |
| ATOM | 1000 | CB | ARG | A | 74 | 16.337 | 9.019 | 54.599 | 1.00 | 8.19 | C |
| ATOM | 1003 | CG | ARG | A | 74 | 16.044 | 9.851 | 55.867 | 1.00 | 9.06 | C |
| ATOM | 1006 | CD | ARG | A | 74 | 14.587 | 9.693 | 56.300 | 1.00 | 8.70 | C |
| ATOM | 1009 | NE | ARG | A | 74 | 14.322 | 8.348 | 56.834 | 1.00 | 9.15 | N |
| ATOM | 1011 | CZ | ARG | A | 74 | 13.146 | 7.711 | 56.777 | 1.00 | 10.02 | C |
| ATOM | 1012 | NH1 | ARG | A | 74 | 12.107 | 8.206 | 56.136 | 1.00 | 8.19 | N |
| ATOM | 1015 | NH2 | ARG | A | 74 | 13.043 | 6.513 | 57.364 | 1.00 | 10.25 | N |
| ATOM | 1018 | C | ARG | A | 74 | 18.640 | 8.368 | 55.231 | 1.00 | 8.94 | C |
| ATOM | 1019 | O | ARG | A | 74 | 18.580 | 7.140 | 55.431 | 1.00 | 8.17 | O |
| ATOM | 1021 | N | THR | A | 75 | 19.362 | 9.187 | 55.991 | 1.00 | 8.60 | N |
| ATOM | 1022 | CA | THR | A | 75 | 20.146 | 8.679 | 57.112 | 1.00 | 8.62 | C |
| ATOM | 1024 | CB | THR | A | 75 | 21.096 | 9.767 | 57.658 | 1.00 | 8.78 | C |
| ATOM | 1026 | OG1 | THR | A | 75 | 20.367 | 10.829 | 58.287 | 1.00 | 8.90 | O |
| ATOM | 1028 | CG2 | THR | A | 75 | 21.959 | 10.364 | 56.585 | 1.00 | 10.28 | C |
| ATOM | 1032 | C | THR | A | 75 | 19.244 | 8.253 | 58.264 | 1.00 | 8.43 | C |
| ATOM | 1033 | O | THR | A | 75 | 18.035 | 8.532 | 58.278 | 1.00 | 8.19 | O |
| ATOM | 1035 | N | GLY | A | 76 | 19.817 | 7.593 | 59.259 | 1.00 | 9.46 | N |
| ATOM | 1036 | CA | GLY | A | 76 | 19.152 | 7.453 | 60.520 | 1.00 | 8.62 | C |
| ATOM | 1039 | C | GLY | A | 76 | 18.957 | 8.781 | 61.212 | 1.00 | 8.90 | C |
| ATOM | 1040 | O | GLY | A | 76 | 19.496 | 9.813 | 60.773 | 1.00 | 8.59 | O |
| ATOM | 1042 | N | TRP | A | 77 | 18.196 | 8.750 | 62.312 | 1.00 | 9.17 | N |
| ATOM | 1043 | CA | TRP | A | 77 | 17.969 | 9.943 | 63.121 | 1.00 | 8.14 | C |
| ATOM | 1045 | CB | TRP | A | 77 | 16.779 | 9.726 | 64.058 | 1.00 | 8.44 | C |
| ATOM | 1048 | CG | TRP | A | 77 | 16.293 | 10.907 | 64.819 | 1.00 | 7.96 | C |
| ATOM | 1049 | CD1 | TRP | A | 77 | 15.678 | 12.024 | 64.318 | 1.00 | 9.29 | C |
| ATOM | 1051 | NE1 | TRP | A | 77 | 15.356 | 12.889 | 65.323 | 1.00 | 8.84 | N |
| ATOM | 1053 | CE2 | TRP | A | 77 | 15.705 | 12.319 | 66.525 | 1.00 | 11.12 | C |
| ATOM | 1054 | CD2 | TRP | A | 77 | 16.302 | 11.073 | 66.245 | 1.00 | 9.62 | C |
| ATOM | 1055 | CE3 | TRP | A | 77 | 16.786 | 10.300 | 67.306 | 1.00 | 10.69 | C |
| ATOM | 1057 | CZ3 | TRP | A | 77 | 16.600 | 10.783 | 68.622 | 1.00 | 10.71 | C |
| ATOM | 1059 | CH2 | TRP | A | 77 | 15.994 | 12.028 | 68.843 | 1.00 | 9.85 | C |
| ATOM | 1061 | CZ2 | TRP | A | 77 | 15.526 | 12.791 | 67.825 | 1.00 | 10.22 | C |
| ATOM | 1063 | C | TRP | A | 77 | 19.232 | 10.253 | 63.938 | 1.00 | 9.50 | C |
| ATOM | 1064 | O | TRP | A | 77 | 19.766 | 9.410 | 64.641 | 1.00 | 10.66 | O |
| ATOM | 1066 | N | VAL | A | 78 | 19.701 | 11.502 | 63.839 | 1.00 | 7.99 | N |
| ATOM | 1067 | CA | VAL | A | 78 | 20.933 | 11.956 | 64.510 | 1.00 | 8.77 | C |
| ATOM | 1069 | CB | VAL | A | 78 | 21.868 | 12.650 | 63.480 | 1.00 | 8.76 | C |
| ATOM | 1071 | CG1 | VAL | A | 78 | 23.149 | 13.128 | 64.160 | 1.00 | 9.73 | C |
| ATOM | 1075 | CG2 | VAL | A | 78 | 22.140 | 11.723 | 62.286 | 1.00 | 10.01 | C |
| ATOM | 1079 | C | VAL | A | 78 | 20.588 | 12.924 | 65.636 | 1.00 | 8.96 | C |
| ATOM | 1080 | O | VAL | A | 78 | 20.182 | 14.033 | 65.385 | 1.00 | 9.98 | O |
| ATOM | 1082 | N | PRO | A | 79 | 20.712 | 12.491 | 66.891 | 1.00 | 10.47 | N |
| ATOM | 1083 | CA | PRO | A | 79 | 20.522 | 13.409 | 68.016 | 1.00 | 11.43 | C |
| ATOM | 1085 | CB | PRO | A | 79 | 20.668 | 12.495 | 69.242 | 1.00 | 12.63 | C |
| ATOM | 1088 | CG | PRO | A | 79 | 20.418 | 11.101 | 68.726 | 1.00 | 12.60 | C |
| ATOM | 1091 | CD | PRO | A | 79 | 20.958 | 11.100 | 67.327 | 1.00 | 12.00 | C |
| ATOM | 1094 | C | PRO | A | 79 | 21.555 | 14.517 | 67.986 | 1.00 | 12.22 | C |
| ATOM | 1095 | O | PRO | A | 79 | 22.738 | 14.224 | 67.784 | 1.00 | 12.62 | O |
| ATOM | 1096 | N | ARG | A | 80 | 21.148 | 15.777 | 68.116 | 1.00 | 12.32 | N |
| ATOM | 1097 | CA | ARG | A | 80 | 22.063 | 16.891 | 67.894 | 1.00 | 15.12 | C |
| ATOM | 1099 | CB | ARG | A | 80 | 21.354 | 18.133 | 67.362 | 1.00 | 15.85 | C |
| ATOM | 1102 | CG | ARG | A | 80 | 20.509 | 18.823 | 68.365 | 1.00 | 15.55 | C |
| ATOM | 1105 | CD | ARG | A | 80 | 20.040 | 20.171 | 67.859 | 1.00 | 16.53 | C |
| ATOM | 1108 | NE | ARG | A | 80 | 19.079 | 20.802 | 68.756 | 1.00 | 16.98 | N |
| ATOM | 1110 | CZ | ARG | A | 80 | 19.358 | 21.768 | 69.639 | 1.00 | 19.02 | C |
| ATOM | 1111 | NH1 | ARG | A | 80 | 20.592 | 22.265 | 69.771 | 1.00 | 18.43 | N |
| ATOM | 1114 | NH2 | ARG | A | 80 | 18.399 | 22.260 | 70.413 | 1.00 | 20.74 | N |
| ATOM | 1117 | C | ARG | A | 80 | 22.889 | 17.306 | 69.114 | 1.00 | 15.78 | C |
| ATOM | 1118 | O | ARG | A | 80 | 23.793 | 18.125 | 68.960 | 1.00 | 15.13 | O |
| ATOM | 1120 | N | ARG | A | 81 | 22.553 | 16.792 | 70.301 | 1.00 | 17.14 | N |
| ATOM | 1121 | CA | ARG | A | 81 | 23.315 | 17.155 | 71.515 | 1.00 | 17.63 | C |
| ATOM | 1123 | CB | ARG | A | 81 | 24.713 | 16.575 | 71.447 | 1.00 | 18.84 | C |
| ATOM | 1126 | CG | ARG | A | 81 | 24.662 | 15.058 | 71.474 | 1.00 | 22.20 | C |
| ATOM | 1129 | CD | ARG | A | 81 | 26.000 | 14.448 | 71.540 | 1.00 | 26.32 | C |
| ATOM | 1132 | NE | ARG | A | 81 | 26.700 | 14.783 | 72.776 | 1.00 | 25.33 | N |
| ATOM | 1134 | CZ | ARG | A | 81 | 28.013 | 14.678 | 72.960 | 1.00 | 28.71 | C |
| ATOM | 1135 | NH1 | ARG | A | 81 | 28.806 | 14.246 | 71.988 | 1.00 | 30.89 | N |
| ATOM | 1138 | NH2 | ARG | A | 81 | 28.543 | 15.029 | 74.121 | 1.00 | 30.52 | N |
| ATOM | 1141 | C | ARG | A | 81 | 23.257 | 18.689 | 71.673 | 1.00 | 18.22 | C |
| ATOM | 1142 | O | ARG | A | 81 | 22.206 | 19.255 | 71.469 | 1.00 | 19.54 | O |
| ATOM | 1144 | N | GLY | A | 82 | 24.336 | 19.375 | 71.993 | 1.00 | 19.11 | N |
| ATOM | 1145 | CA | GLY | A | 82 | 24.162 | 20.830 | 72.148 | 1.00 | 19.74 | C |
| ATOM | 1148 | C | GLY | A | 82 | 24.141 | 21.627 | 70.843 | 1.00 | 18.66 | C |
| ATOM | 1149 | O | GLY | A | 82 | 23.956 | 22.852 | 70.894 | 1.00 | 20.16 | O |
| ATOM | 1151 | N | ALA | A | 83 | 24.300 | 20.958 | 69.699 | 1.00 | 16.48 | N |
| ATOM | 1152 | CA | ALA | A | 83 | 24.749 | 21.647 | 68.486 | 1.00 | 14.41 | C |
| ATOM | 1154 | CB | ALA | A | 83 | 25.109 | 20.650 | 67.433 | 1.00 | 14.82 | C |
| ATOM | 1158 | C | ALA | A | 83 | 23.717 | 22.602 | 67.927 | 1.00 | 15.13 | C |
| ATOM | 1159 | O | ALA | A | 83 | 22.590 | 22.179 | 67.717 | 1.00 | 15.30 | O |
| ATOM | 1161 | N | VAL | A | 84 | 24.099 | 23.840 | 67.619 | 1.00 | 13.70 | N |
| ATOM | 1162 | CA | VAL | A | 84 | 23.208 | 24.783 | 66.949 | 1.00 | 13.96 | C |
| ATOM | 1164 | CB | VAL | A | 84 | 23.285 | 26.201 | 67.506 | 1.00 | 15.82 | C |
| ATOM | 1166 | CG1 | VAL | A | 84 | 22.345 | 27.139 | 66.755 | 1.00 | 16.71 | C |
| ATOM | 1170 | CG2 | VAL | A | 84 | 22.898 | 26.204 | 68.979 | 1.00 | 17.60 | C |
| ATOM | 1174 | C | VAL | A | 84 | 23.456 | 24.739 | 65.425 | 1.00 | 11.78 | C |
| ATOM | 1175 | O | VAL | A | 84 | 22.524 | 24.853 | 64.652 | 1.00 | 11.60 | O |
| ATOM | 1177 | N | HIS | A | 85 | 24.702 | 24.618 | 65.002 | 1.00 | 10.16 | N |
| ATOM | 1178 | CA | HIS | A | 85 | 25.008 | 24.300 | 63.609 | 1.00 | 10.37 | C |
| ATOM | 1180 | CB | HIS | A | 85 | 25.807 | 25.405 | 62.924 | 1.00 | 11.74 | C |
| ATOM | 1183 | CG | HIS | A | 85 | 25.082 | 26.705 | 62.892 | 1.00 | 15.32 | C |
| ATOM | 1184 | ND1 | HIS | A | 85 | 24.291 | 27.100 | 61.828 | 1.00 | 18.93 | N |
| ATOM | 1186 | CE1 | HIS | A | 85 | 23.746 | 28.273 | 62.116 | 1.00 | 13.57 | C |
| ATOM | 1188 | NE2 | HIS | A | 85 | 24.160 | 28.644 | 63.312 | 1.00 | 18.04 | N |
| ATOM | 1190 | CD2 | HIS | A | 85 | 24.979 | 27.670 | 63.827 | 1.00 | 15.62 | C |
| ATOM | 1192 | C | HIS | A | 85 | 25.797 | 23.011 | 63.531 | 1.00 | 9.98 | C |
| ATOM | 1193 | O | HIS | A | 85 | 26.639 | 22.773 | 64.417 | 1.00 | 11.68 | O |
| ATOM | 1195 | N | VAL | A | 86 | 25.484 | 22.197 | 62.533 | 1.00 | 9.78 | N |
| ATOM | 1196 | CA | VAL | A | 86 | 26.186 | 20.955 | 62.280 | 1.00 | 9.44 | C |
| ATOM | 1198 | CB | VAL | A | 86 | 25.225 | 19.738 | 62.406 | 1.00 | 10.60 | C |
| ATOM | 1200 | CG1 | VAL | A | 86 | 25.866 | 18.460 | 61.842 | 1.00 | 12.94 | C |
| ATOM | 1204 | CG2 | VAL | A | 86 | 24.831 | 19.560 | 63.871 | 1.00 | 10.81 | C |
| ATOM | 1208 | C | VAL | A | 86 | 26.823 | 21.029 | 60.908 | 1.00 | 10.03 | C |
| ATOM | 1209 | O | VAL | A | 86 | 26.222 | 21.547 | 59.941 | 1.00 | 11.17 | O |
| ATOM | 1211 | N | TYR | A | 87 | 28.043 | 20.519 | 60.790 | 1.00 | 7.91 | N |
| ATOM | 1212 | CA | TYR | A | 87 | 28.716 | 20.415 | 59.536 | 1.00 | 7.81 | C |
| ATOM | 1214 | CB | TYR | A | 87 | 30.196 | 20.779 | 59.709 | 1.00 | 8.49 | C |
| ATOM | 1217 | CG | TYR | A | 87 | 30.356 | 22.226 | 60.090 | 1.00 | 8.70 | C |
| ATOM | 1218 | CD1 | TYR | A | 87 | 30.439 | 23.212 | 59.131 | 1.00 | 10.52 | C |
| ATOM | 1220 | CE1 | TYR | A | 87 | 30.548 | 24.542 | 59.496 | 1.00 | 11.11 | C |
| ATOM | 1222 | CZ | TYR | A | 87 | 30.511 | 24.876 | 60.820 | 1.00 | 12.13 | C |
| ATOM | 1223 | OH | TYR | A | 87 | 30.616 | 26.203 | 61.247 | 1.00 | 14.33 | O |
| ATOM | 1225 | CE2 | TYR | A | 87 | 30.414 | 23.923 | 61.774 | 1.00 | 10.66 | C |
| ATOM | 1227 | CD2 | TYR | A | 87 | 30.299 | 22.607 | 61.404 | 1.00 | 10.17 | C |
| ATOM | 1229 | C | TYR | A | 87 | 28.605 | 19.024 | 58.963 | 1.00 | 8.80 | C |
| ATOM | 1230 | O | TYR | A | 87 | 28.736 | 18.040 | 59.702 | 1.00 | 9.37 | O |
| ATOM | 1232 | N | ALA | A | 88 | 28.354 | 18.955 | 57.655 | 1.00 | 7.91 | N |
| ATOM | 1233 | CA | ALA | A | 88 | 28.258 | 17.688 | 56.937 | 1.00 | 7.02 | C |
| ATOM | 1235 | CB | ALA | A | 88 | 26.853 | 17.549 | 56.298 | 1.00 | 7.55 | C |
| ATOM | 1239 | C | ALA | A | 88 | 29.270 | 17.584 | 55.830 | 1.00 | 8.97 | C |
| ATOM | 1240 | O | ALA | A | 88 | 29.123 | 18.283 | 54.818 | 1.00 | 10.24 | O |
| ATOM | 1242 | N | THR | A | 89 | 30.290 | 16.765 | 55.996 | 1.00 | 8.71 | N |
| ATOM | 1243 | CA | THR | A | 89 | 31.290 | 16.569 | 54.944 | 1.00 | 7.77 | C |
| ATOM | 1245 | CB | THR | A | 89 | 32.686 | 16.393 | 55.511 | 1.00 | 8.61 | C |
| ATOM | 1247 | OG1 | THR | A | 89 | 33.092 | 17.561 | 56.259 | 1.00 | 9.57 | O |
| ATOM | 1249 | CG2 | THR | A | 89 | 33.675 | 16.151 | 54.393 | 1.00 | 8.17 | C |
| ATOM | 1253 | C | THR | A | 89 | 30.899 | 15.346 | 54.149 | 1.00 | 8.06 | C |
| ATOM | 1254 | O | THR | A | 89 | 30.762 | 14.265 | 54.682 | 1.00 | 8.00 | O |
| ATOM | 1256 | N | LEU | A | 90 | 30.700 | 15.571 | 52.842 | 1.00 | 8.21 | N |
| ATOM | 1257 | CA | LEU | A | 90 | 30.340 | 14.538 | 51.869 | 1.00 | 8.69 | C |
| ATOM | 1259 | CB | LEU | A | 90 | 29.139 | 15.023 | 51.019 | 1.00 | 9.11 | C |
| ATOM | 1262 | CG | LEU | A | 90 | 27.861 | 15.331 | 51.822 | 1.00 | 15.79 | C |
| ATOM | 1264 | CD1 | LEU | A | 90 | 26.896 | 16.070 | 50.900 | 1.00 | 20.74 | C |
| ATOM | 1268 | CD2 | LEU | A | 90 | 27.283 | 14.096 | 52.289 | 1.00 | 19.77 | C |
| ATOM | 1272 | C | LEU | A | 90 | 31.508 | 14.291 | 50.920 | 1.00 | 8.92 | C |
| ATOM | 1273 | O | LEU | A | 90 | 32.029 | 15.245 | 50.367 | 1.00 | 10.39 | O |
| ATOM | 1275 | N | ARG | A | 91 | 31.888 | 13.034 | 50.722 | 1.00 | 8.77 | N |
| ATOM | 1276 | CA | ARG | A | 91 | 32.911 | 12.662 | 49.732 | 1.00 | 8.68 | C |
| ATOM | 1278 | CB | ARG | A | 91 | 34.142 | 12.014 | 50.403 | 1.00 | 9.59 | C |
| ATOM | 1281 | CG | ARG | A | 91 | 34.773 | 12.963 | 51.396 | 1.00 | 9.66 | C |
| ATOM | 1284 | CD | ARG | A | 91 | 36.013 | 12.436 | 52.061 | 1.00 | 11.36 | C |
| ATOM | 1287 | NE | ARG | A | 91 | 36.469 | 13.307 | 53.142 | 1.00 | 11.87 | N |
| ATOM | 1289 | CZ | ARG | A | 91 | 37.207 | 14.409 | 52.998 | 1.00 | 10.22 | C |
| ATOM | 1290 | NH1 | ARG | A | 91 | 37.516 | 15.118 | 54.108 | 1.00 | 12.10 | N |
| ATOM | 1293 | NH2 | ARG | A | 91 | 37.708 | 14.777 | 51.815 | 1.00 | 12.98 | N |
| ATOM | 1296 | C | ARG | A | 91 | 32.241 | 11.759 | 48.729 | 1.00 | 9.26 | C |
| ATOM | 1297 | O | ARG | A | 91 | 31.544 | 10.796 | 49.105 | 1.00 | 8.93 | O |
| ATOM | 1299 | N | PHE | A | 92 | 32.415 | 12.050 | 47.438 | 1.00 | 7.97 | N |
| ATOM | 1300 | CA | PHE | A | 92 | 31.661 | 11.373 | 46.418 | 1.00 | 6.88 | C |
| ATOM | 1302 | CB | PHE | A | 92 | 30.239 | 12.000 | 46.308 | 1.00 | 7.83 | C |
| ATOM | 1305 | CG | PHE | A | 92 | 30.221 | 13.431 | 45.837 | 1.00 | 7.27 | C |
| ATOM | 1306 | CD1 | PHE | A | 92 | 30.056 | 13.745 | 44.471 | 1.00 | 8.11 | C |
| ATOM | 1308 | CE1 | PHE | A | 92 | 30.042 | 15.066 | 44.056 | 1.00 | 8.18 | C |
| ATOM | 1310 | CZ | PHE | A | 92 | 30.219 | 16.063 | 44.939 | 1.00 | 8.65 | C |
| ATOM | 1312 | CE2 | PHE | A | 92 | 30.397 | 15.799 | 46.280 | 1.00 | 9.28 | C |
| ATOM | 1314 | CD2 | PHE | A | 92 | 30.359 | 14.489 | 46.736 | 1.00 | 8.23 | C |
| ATOM | 1316 | C | PHE | A | 92 | 32.347 | 11.364 | 45.069 | 1.00 | 7.47 | C |
| ATOM | 1317 | O | PHE | A | 92 | 33.136 | 12.245 | 44.721 | 1.00 | 8.15 | O |
| ATOM | 1319 | N | THR | A | 93 | 32.029 | 10.327 | 44.315 | 1.00 | 7.11 | N |
| ATOM | 1320 | CA | THR | A | 93 | 32.417 | 10.218 | 42.886 | 1.00 | 7.40 | C |
| ATOM | 1322 | CB | ATHR | A | 93 | 33.136 | 8.982 | 42.504 | 0.50 | 8.62 | C |
| ATOM | 1323 | CB | BTHR | A | 93 | 32.705 | 8.657 | 42.619 | 0.50 | 7.27 | C |
| ATOM | 1326 | OG1 | ATHR | A | 93 | 32.308 | 7.887 | 42.774 | 0.50 | 10.04 | O |
| ATOM | 1327 | OG1 | BTHR | A | 93 | 33.752 | 8.147 | 43.485 | 0.50 | 7.05 | O |
| ATOM | 1330 | CG2 | ATHR | A | 93 | 34.452 | 8.867 | 43.335 | 0.50 | 6.07 | C |
| ATOM | 1331 | CG2 | BTHR | A | 93 | 33.038 | 8.353 | 41.171 | 0.50 | 6.94 | C |
| ATOM | 1338 | C | THR | A | 93 | 31.212 | 10.559 | 42.044 | 1.00 | 7.68 | C |
| ATOM | 1339 | O | THR | A | 93 | 30.073 | 10.205 | 42.401 | 1.00 | 7.10 | O |
| ATOM | 1341 | N | MET | A | 94 | 31.442 | 11.283 | 40.954 | 1.00 | 7.62 | N |
| ATOM | 1342 | CA | MET | A | 94 | 30.404 | 11.649 | 39.987 | 1.00 | 6.97 | C |
| ATOM | 1344 | CB | MET | A | 94 | 30.119 | 13.136 | 40.070 | 1.00 | 7.97 | C |
| ATOM | 1347 | CG | MET | A | 94 | 28.937 | 13.593 | 39.231 | 1.00 | 9.97 | C |
| ATOM | 1350 | SD | MET | A | 94 | 27.301 | 12.983 | 39.741 | 1.00 | 9.34 | S |
| ATOM | 1351 | CE | MET | A | 94 | 27.147 | 13.874 | 41.283 | 1.00 | 10.18 | C |
| ATOM | 1355 | C | MET | A | 94 | 30.877 | 11.291 | 38.588 | 1.00 | 7.52 | C |
| ATOM | 1356 | O | MET | A | 94 | 31.958 | 11.760 | 38.167 | 1.00 | 7.31 | O |
| ATOM | 1358 | N | LEU | A | 95 | 30.152 | 10.399 | 37.907 | 1.00 | 7.87 | N |
| ATOM | 1359 | CA | LEU | A | 95 | 30.511 | 10.025 | 36.542 | 1.00 | 7.80 | C |
| ATOM | 1361 | CB | LEU | A | 95 | 29.836 | 8.714 | 36.120 | 1.00 | 7.80 | C |
| ATOM | 1364 | CG | LEU | A | 95 | 30.586 | 7.423 | 36.437 | 1.00 | 9.86 | C |
| ATOM | 1366 | CD1 | LEU | A | 95 | 31.820 | 7.207 | 35.574 | 1.00 | 11.31 | C |
| ATOM | 1370 | CD2 | LEU | A | 95 | 30.907 | 7.368 | 37.943 | 1.00 | 10.48 | C |
| ATOM | 1374 | C | LEU | A | 95 | 30.176 | 11.134 | 35.520 | 1.00 | 9.01 | C |
| ATOM | 1375 | O | LEU | A | 95 | 29.127 | 11.817 | 35.573 | 1.00 | 9.51 | O |
| ATOM | 1377 | N | GLU | A | 96 | 31.105 | 11.287 | 34.588 | 1.00 | 7.72 | N |
| ATOM | 1378 | CA | GLU | A | 96 | 30.885 | 12.081 | 33.378 | 1.00 | 9.25 | C |
| ATOM | 1380 | CB | GLU | A | 96 | 32.194 | 12.109 | 32.576 | 1.00 | 9.27 | C |
| ATOM | 1383 | CG | GLU | A | 96 | 32.102 | 12.995 | 31.340 | 1.00 | 11.60 | C |
| ATOM | 1386 | CD | GLU | A | 96 | 33.104 | 12.676 | 30.232 | 1.00 | 12.47 | C |
| ATOM | 1387 | OE1 | GLU | A | 96 | 33.622 | 11.523 | 30.087 | 1.00 | 15.75 | O |
| ATOM | 1388 | OE2 | GLU | A | 96 | 33.299 | 13.608 | 29.422 | 1.00 | 19.07 | O |
| ATOM | 1389 | C | GLU | A | 96 | 29.757 | 11.441 | 32.528 | 1.00 | 8.51 | C |
| ATOM | 1390 | O | GLU | A | 96 | 29.860 | 10.294 | 32.115 | 1.00 | 8.64 | O |
| ATOM | 1392 | N | CYS | A | 97 | 28.667 | 12.175 | 32.302 | 1.00 | 9.62 | N |
| ATOM | 1393 | CA | CYS | A | 97 | 27.535 | 11.669 | 31.529 | 1.00 | 9.42 | C |
| ATOM | 1395 | CB | CYS | A | 97 | 26.483 | 12.790 | 31.349 | 1.00 | 8.87 | C |
| ATOM | 1398 | SG | CYS | A | 97 | 25.542 | 13.065 | 32.851 | 1.00 | 12.10 | S |
| ATOM | 1400 | C | CYS | A | 97 | 27.963 | 11.127 | 30.161 | 1.00 | 9.98 | C |
| ATOM | 1401 | O | CYS | A | 97 | 27.520 | 10.056 | 29.716 | 1.00 | 9.98 | O |
| ATOM | 1403 | N | LEU | A | 98 | 28.855 | 11.850 | 29.511 | 1.00 | 9.65 | N |
| ATOM | 1404 | CA | LEU | A | 98 | 29.404 | 11.373 | 28.223 | 1.00 | 11.52 | C |
| ATOM | 1406 | CB | LEU | A | 98 | 30.241 | 12.452 | 27.597 | 1.00 | 12.26 | C |
| ATOM | 1409 | CG | LEU | A | 98 | 29.420 | 13.622 | 27.039 | 1.00 | 15.94 | C |
| ATOM | 1411 | CD1 | LEU | A | 98 | 30.349 | 14.683 | 26.518 | 1.00 | 19.86 | C |
| ATOM | 1415 | CD2 | LEU | A | 98 | 28.428 | 13.204 | 25.939 | 1.00 | 19.10 | C |
| ATOM | 1419 | C | LEU | A | 98 | 30.160 | 10.023 | 28.230 | 1.00 | 10.56 | C |
| ATOM | 1420 | O | LEU | A | 98 | 30.307 | 9.407 | 27.174 | 1.00 | 10.78 | O |
| ATOM | 1422 | N | SER | A | 99 | 30.649 | 9.586 | 29.387 | 1.00 | 9.83 | N |
| ATOM | 1423 | CA | SER | A | 99 | 31.226 | 8.259 | 29.520 | 1.00 | 9.91 | C |
| ATOM | 1425 | CB | SER | A | 99 | 32.175 | 8.171 | 30.729 | 1.00 | 10.76 | C |
| ATOM | 1428 | OG | SER | A | 99 | 33.364 | 8.855 | 30.520 | 1.00 | 11.45 | O |
| ATOM | 1430 | C | SER | A | 99 | 30.230 | 7.132 | 29.658 | 1.00 | 11.32 | C |
| ATOM | 1431 | O | SER | A | 99 | 30.641 | 5.970 | 29.671 | 1.00 | 12.39 | O |
| ATOM | 1433 | N | LEU | A | 100 | 28.943 | 7.438 | 29.831 | 1.00 | 9.82 | N |
| ATOM | 1434 | CA | LEU | A | 100 | 27.958 | 6.400 | 30.119 | 1.00 | 10.93 | C |
| ATOM | 1436 | CB | LEU | A | 100 | 26.996 | 6.915 | 31.204 | 1.00 | 10.49 | C |
| ATOM | 1439 | CG | LEU | A | 100 | 27.629 | 7.403 | 32.517 | 1.00 | 11.90 | C |
| ATOM | 1441 | CD1 | LEU | A | 100 | 26.615 | 8.112 | 33.387 | 1.00 | 12.68 | C |
| ATOM | 1445 | CD2 | LEU | A | 100 | 28.298 | 6.256 | 33.253 | 1.00 | 12.67 | C |
| ATOM | 1449 | C | LEU | A | 100 | 27.199 | 6.043 | 28.858 | 1.00 | 10.94 | C |
| ATOM | 1450 | O | LEU | A | 100 | 26.527 | 6.895 | 28.300 | 1.00 | 11.49 | O |
| ATOM | 1452 | N | PRO | A | 101 | 27.328 | 4.802 | 28.376 | 1.00 | 12.49 | N |
| ATOM | 1453 | CA | PRO | A | 101 | 26.705 | 4.491 | 27.079 | 1.00 | 14.38 | C |
| ATOM | 1455 | CB | PRO | A | 101 | 27.164 | 3.049 | 26.780 | 1.00 | 14.56 | C |
| ATOM | 1458 | CG | PRO | A | 101 | 27.671 | 2.504 | 28.043 | 1.00 | 13.39 | C |
| ATOM | 1461 | CD | PRO | A | 101 | 28.072 | 3.664 | 28.932 | 1.00 | 12.86 | C |
| ATOM | 1464 | C | PRO | A | 101 | 25.179 | 4.618 | 27.170 | 1.00 | 16.17 | C |
| ATOM | 1465 | O | PRO | A | 101 | 24.586 | 4.219 | 28.169 | 1.00 | 17.28 | O |
| ATOM | 1466 | N | ARG | A | 102 | 24.542 | 5.236 | 26.180 | 1.00 | 18.27 | N |
| ATOM | 1467 | CA | ARG | A | 102 | 23.071 | 5.467 | 26.252 | 1.00 | 18.80 | C |
| ATOM | 1469 | CB | ARG | A | 102 | 22.288 | 4.167 | 26.058 | 1.00 | 20.13 | C |
| ATOM | 1478 | C | ARG | A | 102 | 22.501 | 6.186 | 27.484 | 1.00 | 20.78 | C |
| ATOM | 1479 | O | ARG | A | 102 | 21.292 | 6.102 | 27.722 | 1.00 | 20.36 | O |
| ATOM | 1481 | N | ALA | A | 103 | 23.344 | 6.848 | 28.278 | 1.00 | 21.29 | N |
| ATOM | 1482 | CA | ALA | A | 103 | 22.902 | 8.075 | 28.944 | 1.00 | 22.02 | C |
| ATOM | 1484 | CB | ALA | A | 103 | 24.018 | 8.713 | 29.749 | 1.00 | 21.04 | C |
| ATOM | 1488 | C | ALA | A | 103 | 22.504 | 8.998 | 27.806 | 1.00 | 23.53 | C |
| ATOM | 1489 | O | ALA | A | 103 | 23.080 | 8.919 | 26.705 | 1.00 | 26.11 | O |
| ATOM | 1491 | N | GLY | A | 104 | 21.536 | 9.860 | 28.046 | 1.00 | 24.21 | N |
| ATOM | 1492 | CA | GLY | A | 104 | 21.126 | 10.821 | 27.038 | 1.00 | 24.18 | C |
| ATOM | 1495 | C | GLY | A | 104 | 21.141 | 12.226 | 27.582 | 1.00 | 24.08 | C |
| ATOM | 1496 | O | GLY | A | 104 | 21.794 | 12.520 | 28.615 | 1.00 | 24.73 | O |
| ATOM | 1498 | N | ARG | A | 105 | 20.363 | 13.077 | 26.925 | 1.00 | 23.02 | N |
| ATOM | 1499 | CA | ARG | A | 105 | 20.319 | 14.505 | 27.210 | 1.00 | 21.51 | C |
| ATOM | 1501 | CB | ARG | A | 105 | 19.374 | 15.210 | 26.220 | 1.00 | 22.65 | C |
| ATOM | 1510 | C | ARG | A | 105 | 19.897 | 14.834 | 28.654 | 1.00 | 20.55 | C |
| ATOM | 1511 | O | ARG | A | 105 | 20.291 | 15.867 | 29.153 | 1.00 | 21.64 | O |
| ATOM | 1513 | N | SER | A | 106 | 19.133 | 13.946 | 29.300 | 1.00 | 18.47 | N |
| ATOM | 1514 | CA | SER | A | 106 | 18.572 | 14.125 | 30.627 | 1.00 | 18.10 | C |
| ATOM | 1516 | CB | SER | A | 106 | 17.404 | 13.135 | 30.738 | 1.00 | 17.99 | C |
| ATOM | 1519 | OG | SER | A | 106 | 16.607 | 13.369 | 31.835 | 1.00 | 25.30 | O |
| ATOM | 1521 | C | SER | A | 106 | 19.563 | 13.852 | 31.795 | 1.00 | 14.68 | C |
| ATOM | 1522 | O | SER | A | 106 | 19.290 | 14.115 | 32.987 | 1.00 | 15.45 | O |
| ATOM | 1524 | N | CYS | A | 107 | 20.708 | 13.282 | 31.443 | 1.00 | 12.87 | N |
| ATOM | 1525 | CA | CYS | A | 107 | 21.700 | 12.891 | 32.455 | 1.00 | 11.12 | C |
| ATOM | 1527 | CB | CYS | A | 107 | 22.817 | 12.123 | 31.753 | 1.00 | 11.59 | C |
| ATOM | 1530 | SG | CYS | A | 107 | 24.162 | 11.512 | 32.802 | 1.00 | 12.27 | S |
| ATOM | 1532 | C | CYS | A | 107 | 22.221 | 14.143 | 33.175 | 1.00 | 10.83 | C |
| ATOM | 1533 | O | CYS | A | 107 | 22.444 | 15.198 | 32.548 | 1.00 | 11.33 | O |
| ATOM | 1535 | N | LYS | A | 108 | 22.466 | 14.000 | 34.485 | 1.00 | 10.91 | N |
| ATOM | 1536 | CA | LYS | A | 108 | 22.880 | 15.103 | 35.357 | 1.00 | 10.39 | C |
| ATOM | 1538 | CB | LYS | A | 108 | 21.810 | 15.383 | 36.426 | 1.00 | 11.64 | C |
| ATOM | 1541 | CG | LYS | A | 108 | 20.441 | 15.745 | 35.847 | 1.00 | 14.77 | C |
| ATOM | 1544 | CD | LYS | A | 108 | 20.525 | 16.934 | 34.961 | 1.00 | 17.33 | C |
| ATOM | 1547 | CE | LYS | A | 108 | 19.159 | 17.265 | 34.350 | 1.00 | 19.39 | C |
| ATOM | 1550 | NZ | LYS | A | 108 | 19.274 | 18.369 | 33.346 | 1.00 | 25.41 | N |
| ATOM | 1554 | C | LYS | A | 108 | 24.195 | 14.732 | 36.042 | 1.00 | 9.99 | C |
| ATOM | 1555 | O | LYS | A | 108 | 24.544 | 13.535 | 36.099 | 1.00 | 9.47 | O |
| ATOM | 1557 | N | GLU | A | 109 | 24.893 | 15.755 | 36.537 | 1.00 | 9.09 | N |
| ATOM | 1558 | CA | GLU | A | 109 | 26.160 | 15.592 | 37.286 | 1.00 | 8.94 | C |
| ATOM | 1560 | CB | GLU | A | 109 | 27.343 | 16.058 | 36.435 | 1.00 | 9.45 | C |
| ATOM | 1563 | CG | GLU | A | 109 | 27.565 | 15.126 | 35.214 | 1.00 | 9.12 | C |
| ATOM | 1566 | CD | GLU | A | 109 | 28.416 | 15.737 | 34.096 | 1.00 | 11.55 | C |
| ATOM | 1567 | OE1 | GLU | A | 109 | 28.725 | 16.969 | 34.146 | 1.00 | 12.46 | O |
| ATOM | 1568 | OE2 | GLU | A | 109 | 28.695 | 15.006 | 33.114 | 1.00 | 11.28 | O |
| ATOM | 1569 | C | GLU | A | 109 | 26.107 | 16.264 | 38.660 | 1.00 | 9.52 | C |
| ATOM | 1570 | O | GLU | A | 109 | 27.103 | 16.795 | 39.154 | 1.00 | 9.16 | O |
| ATOM | 1572 | N | THR | A | 110 | 24.926 | 16.208 | 39.265 | 1.00 | 9.72 | N |
| ATOM | 1573 | CA | THR | A | 110 | 24.634 | 16.848 | 40.560 | 1.00 | 10.44 | C |
| ATOM | 1575 | CB | THR | A | 110 | 23.915 | 18.228 | 40.417 | 1.00 | 11.43 | C |
| ATOM | 1577 | OG1 | THR | A | 110 | 22.567 | 18.038 | 39.910 | 1.00 | 11.72 | O |
| ATOM | 1579 | CG2 | THR | A | 110 | 24.671 | 19.176 | 39.515 | 1.00 | 12.59 | C |
| ATOM | 1583 | C | THR | A | 110 | 23.711 | 15.949 | 41.378 | 1.00 | 10.11 | C |
| ATOM | 1584 | O | THR | A | 110 | 23.154 | 14.985 | 40.854 | 1.00 | 9.27 | O |
| ATOM | 1586 | N | PHE | A | 111 | 23.621 | 16.248 | 42.666 | 1.00 | 9.89 | N |
| ATOM | 1587 | CA | PHE | A | 111 | 22.630 | 15.628 | 43.565 | 1.00 | 9.35 | C |
| ATOM | 1589 | CB | PHE | A | 111 | 23.124 | 14.385 | 44.258 | 1.00 | 9.32 | C |
| ATOM | 1592 | CG | PHE | A | 111 | 24.261 | 14.625 | 45.235 | 1.00 | 9.36 | C |
| ATOM | 1593 | CD1 | PHE | A | 111 | 24.050 | 14.561 | 46.582 | 1.00 | 11.62 | C |
| ATOM | 1595 | CE1 | PHE | A | 111 | 25.090 | 14.724 | 47.466 | 1.00 | 11.92 | C |
| ATOM | 1597 | CZ | PHE | A | 111 | 26.381 | 14.950 | 47.011 | 1.00 | 11.03 | C |
| ATOM | 1599 | CE2 | PHE | A | 111 | 26.618 | 15.013 | 45.701 | 1.00 | 10.10 | C |
| ATOM | 1601 | CD2 | PHE | A | 111 | 25.569 | 14.828 | 44.782 | 1.00 | 9.30 | C |
| ATOM | 1603 | C | PHE | A | 111 | 22.240 | 16.729 | 44.561 | 1.00 | 10.26 | C |
| ATOM | 1604 | O | PHE | A | 111 | 22.954 | 17.724 | 44.726 | 1.00 | 9.99 | O |
| ATOM | 1606 | N | THR | A | 112 | 21.090 | 16.576 | 45.199 | 1.00 | 8.76 | N |
| ATOM | 1607 | CA | THR | A | 112 | 20.624 | 17.601 | 46.133 | 1.00 | 8.93 | C |
| ATOM | 1609 | CB | THR | A | 112 | 19.291 | 18.185 | 45.671 | 1.00 | 10.01 | C |
| ATOM | 1611 | OG1 | THR | A | 112 | 19.472 | 18.700 | 44.324 | 1.00 | 10.82 | O |
| ATOM | 1613 | CG2 | THR | A | 112 | 18.818 | 19.278 | 46.593 | 1.00 | 10.35 | C |
| ATOM | 1617 | C | THR | A | 112 | 20.554 | 17.039 | 47.567 | 1.00 | 8.49 | C |
| ATOM | 1618 | O | THR | A | 112 | 20.090 | 15.914 | 47.811 | 1.00 | 9.37 | O |
| ATOM | 1620 | N | VAL | A | 113 | 21.025 | 17.864 | 48.492 | 1.00 | 7.91 | N |
| ATOM | 1621 | CA | VAL | A | 113 | 21.098 | 17.515 | 49.910 | 1.00 | 8.28 | C |
| ATOM | 1623 | CB | VAL | A | 113 | 22.472 | 17.809 | 50.495 | 1.00 | 9.20 | C |
| ATOM | 1625 | CG1 | VAL | A | 113 | 22.472 | 17.446 | 52.027 | 1.00 | 10.87 | C |
| ATOM | 1629 | CG2 | VAL | A | 113 | 23.518 | 16.997 | 49.742 | 1.00 | 9.25 | C |
| ATOM | 1633 | C | VAL | A | 113 | 20.042 | 18.337 | 50.668 | 1.00 | 9.30 | C |
| ATOM | 1634 | O | VAL | A | 113 | 19.916 | 19.559 | 50.455 | 1.00 | 8.34 | O |
| ATOM | 1636 | N | PHE | A | 114 | 19.287 | 17.628 | 51.522 | 1.00 | 8.22 | N |
| ATOM | 1637 | CA | PHE | A | 114 | 18.233 | 18.186 | 52.384 | 1.00 | 8.86 | C |
| ATOM | 1639 | CB | PHE | A | 114 | 16.847 | 17.662 | 51.976 | 1.00 | 9.77 | C |
| ATOM | 1642 | CG | PHE | A | 114 | 16.447 | 17.939 | 50.536 | 1.00 | 9.99 | C |
| ATOM | 1643 | CD1 | PHE | A | 114 | 15.627 | 19.029 | 50.213 | 1.00 | 10.02 | C |
| ATOM | 1645 | CE1 | PHE | A | 114 | 15.245 | 19.261 | 48.927 | 1.00 | 9.70 | C |
| ATOM | 1647 | CZ | PHE | A | 114 | 15.702 | 18.393 | 47.902 | 1.00 | 9.70 | C |
| ATOM | 1649 | CE2 | PHE | A | 114 | 16.454 | 17.308 | 48.214 | 1.00 | 10.86 | C |
| ATOM | 1651 | CD2 | PHE | A | 114 | 16.851 | 17.081 | 49.512 | 1.00 | 9.83 | C |
| ATOM | 1653 | C | PHE | A | 114 | 18.402 | 17.739 | 53.835 | 1.00 | 8.96 | C |
| ATOM | 1654 | O | PHE | A | 114 | 19.070 | 16.721 | 54.119 | 1.00 | 8.46 | O |
| ATOM | 1656 | N | TYR | A | 115 | 17.785 | 18.447 | 54.763 | 1.00 | 8.63 | N |
| ATOM | 1657 | CA | TYR | A | 115 | 17.628 | 17.889 | 56.116 | 1.00 | 8.52 | C |
| ATOM | 1659 | CB | TYR | A | 115 | 18.717 | 18.352 | 57.092 | 1.00 | 8.85 | C |
| ATOM | 1662 | CG | TYR | A | 115 | 18.585 | 19.788 | 57.592 | 1.00 | 8.06 | C |
| ATOM | 1663 | CD1 | TYR | A | 115 | 18.116 | 20.064 | 58.861 | 1.00 | 10.86 | C |
| ATOM | 1665 | CE1 | TYR | A | 115 | 17.978 | 21.382 | 59.294 | 1.00 | 11.45 | C |
| ATOM | 1667 | CZ | TYR | A | 115 | 18.312 | 22.416 | 58.461 | 1.00 | 12.69 | C |
| ATOM | 1668 | OH | TYR | A | 115 | 18.216 | 23.747 | 58.908 | 1.00 | 15.40 | O |
| ATOM | 1670 | CE2 | TYR | A | 115 | 18.818 | 22.152 | 57.233 | 1.00 | 12.22 | C |
| ATOM | 1672 | CD2 | TYR | A | 115 | 18.906 | 20.837 | 56.791 | 1.00 | 8.82 | C |
| ATOM | 1674 | C | TYR | A | 115 | 16.241 | 18.195 | 56.674 | 1.00 | 7.78 | C |
| ATOM | 1675 | O | TYR | A | 115 | 15.548 | 19.067 | 56.162 | 1.00 | 8.01 | O |
| ATOM | 1677 | N | TYR | A | 116 | 15.862 | 17.463 | 57.721 | 1.00 | 8.13 | N |
| ATOM | 1678 | CA | TYR | A | 116 | 14.605 | 17.718 | 58.413 | 1.00 | 8.62 | C |
| ATOM | 1680 | CB | TYR | A | 116 | 13.434 | 16.894 | 57.841 | 1.00 | 8.49 | C |
| ATOM | 1683 | CG | TYR | A | 116 | 12.137 | 17.248 | 58.540 | 1.00 | 9.29 | C |
| ATOM | 1684 | CD1 | TYR | A | 116 | 11.603 | 18.513 | 58.398 | 1.00 | 7.91 | C |
| ATOM | 1686 | CE1 | TYR | A | 116 | 10.440 | 18.911 | 59.091 | 1.00 | 9.45 | C |
| ATOM | 1688 | CZ | TYR | A | 116 | 9.848 | 18.035 | 59.962 | 1.00 | 11.48 | C |
| ATOM | 1689 | OH | TYR | A | 116 | 8.746 | 18.469 | 60.719 | 1.00 | 14.30 | O |
| ATOM | 1691 | CE2 | TYR | A | 116 | 10.383 | 16.747 | 60.125 | 1.00 | 11.31 | C |
| ATOM | 1693 | CD2 | TYR | A | 116 | 11.542 | 16.394 | 59.459 | 1.00 | 12.12 | C |
| ATOM | 1695 | C | TYR | A | 116 | 14.813 | 17.407 | 59.897 | 1.00 | 8.75 | C |
| ATOM | 1696 | O | TYR | A | 116 | 15.348 | 16.352 | 60.233 | 1.00 | 8.64 | O |
| ATOM | 1698 | N | GLU | A | 117 | 14.455 | 18.344 | 60.768 | 1.00 | 9.01 | N |
| ATOM | 1699 | CA | GLU | A | 117 | 14.596 | 18.131 | 62.196 | 1.00 | 9.67 | C |
| ATOM | 1701 | CB | GLU | A | 117 | 14.807 | 19.445 | 62.934 | 1.00 | 9.99 | C |
| ATOM | 1704 | CG | GLU | A | 117 | 15.905 | 20.323 | 62.447 | 1.00 | 11.48 | C |
| ATOM | 1707 | CD | GLU | A | 117 | 16.063 | 21.503 | 63.381 | 1.00 | 13.75 | C |
| ATOM | 1708 | OE1 | GLU | A | 117 | 16.333 | 21.243 | 64.577 | 1.00 | 13.79 | O |
| ATOM | 1709 | OE2 | GLU | A | 117 | 15.902 | 22.651 | 62.901 | 1.00 | 17.99 | O |
| ATOM | 1710 | C | GLU | A | 117 | 13.331 | 17.504 | 62.789 | 1.00 | 9.86 | C |
| ATOM | 1711 | O | GLU | A | 117 | 12.212 | 17.874 | 62.410 | 1.00 | 10.42 | O |
| ATOM | 1713 | N | SER | A | 118 | 13.478 | 16.574 | 63.737 | 1.00 | 8.88 | N |
| ATOM | 1714 | CA | SER | A | 118 | 12.326 | 15.959 | 64.396 | 1.00 | 9.11 | C |
| ATOM | 1716 | CB | SER | A | 118 | 11.879 | 14.659 | 63.724 | 1.00 | 9.48 | C |
| ATOM | 1719 | OG | SER | A | 118 | 12.881 | 13.657 | 63.864 | 1.00 | 10.18 | O |
| ATOM | 1721 | C | SER | A | 118 | 12.681 | 15.716 | 65.875 | 1.00 | 8.87 | C |
| ATOM | 1722 | O | SER | A | 118 | 13.854 | 15.513 | 66.202 | 1.00 | 8.37 | O |
| ATOM | 1724 | N | ASP | A | 119 | 11.688 | 15.741 | 66.762 | 1.00 | 8.36 | N |
| ATOM | 1725 | CA | ASP | A | 119 | 11.956 | 15.650 | 68.203 | 1.00 | 8.09 | C |
| ATOM | 1727 | CB | ASP | A | 119 | 10.870 | 16.386 | 69.021 | 1.00 | 8.58 | C |
| ATOM | 1730 | CG | ASP | A | 119 | 11.244 | 17.852 | 69.321 | 1.00 | 9.08 | C |
| ATOM | 1731 | OD1 | ASP | A | 119 | 12.453 | 18.141 | 69.540 | 1.00 | 10.16 | O |
| ATOM | 1732 | OD2 | ASP | A | 119 | 10.325 | 18.697 | 69.394 | 1.00 | 9.56 | O |
| ATOM | 1733 | C | ASP | A | 119 | 12.159 | 14.204 | 68.678 | 1.00 | 7.79 | C |
| ATOM | 1734 | O | ASP | A | 119 | 12.595 | 13.945 | 69.818 | 1.00 | 9.62 | O |
| ATOM | 1736 | N | ALA | A | 120 | 11.912 | 13.243 | 67.779 | 1.00 | 7.59 | N |
| ATOM | 1737 | CA | ALA | A | 120 | 12.149 | 11.813 | 68.070 | 1.00 | 7.40 | C |
| ATOM | 1739 | CB | ALA | A | 120 | 10.978 | 11.212 | 68.854 | 1.00 | 8.33 | C |
| ATOM | 1743 | C | ALA | A | 120 | 12.326 | 11.074 | 66.768 | 1.00 | 8.16 | C |
| ATOM | 1744 | O | ALA | A | 120 | 12.084 | 11.616 | 65.707 | 1.00 | 8.25 | O |
| ATOM | 1746 | N | ASP | A | 121 | 12.737 | 9.810 | 66.857 | 1.00 | 8.59 | N |
| ATOM | 1747 | CA | ASP | A | 121 | 13.030 | 9.034 | 65.648 | 1.00 | 8.52 | C |
| ATOM | 1749 | CB | ASP | A | 121 | 14.105 | 8.003 | 65.918 | 1.00 | 8.73 | C |
| ATOM | 1752 | CG | ASP | A | 121 | 14.467 | 7.170 | 64.703 | 1.00 | 10.54 | C |
| ATOM | 1753 | OD1 | ASP | A | 121 | 14.060 | 7.468 | 63.530 | 1.00 | 10.18 | O |
| ATOM | 1754 | OD2 | ASP | A | 121 | 15.194 | 6.127 | 64.922 | 1.00 | 11.89 | O |
| ATOM | 1755 | C | ASP | A | 121 | 11.746 | 8.379 | 65.182 | 1.00 | 8.94 | C |
| ATOM | 1756 | O | ASP | A | 121 | 11.482 | 7.176 | 65.386 | 1.00 | 10.31 | O |
| ATOM | 1758 | N | THR | A | 122 | 10.956 | 9.209 | 64.516 | 1.00 | 8.26 | N |
| ATOM | 1759 | CA | THR | A | 122 | 9.590 | 8.879 | 64.128 | 1.00 | 8.62 | C |
| ATOM | 1761 | CB | THR | A | 122 | 8.654 | 10.049 | 64.524 | 1.00 | 9.55 | C |
| ATOM | 1763 | OG1 | THR | A | 122 | 9.250 | 11.295 | 64.131 | 1.00 | 10.04 | O |
| ATOM | 1765 | CG2 | THR | A | 122 | 8.384 | 10.050 | 66.013 | 1.00 | 12.30 | C |
| ATOM | 1769 | C | THR | A | 122 | 9.413 | 8.606 | 62.633 | 1.00 | 8.96 | C |
| ATOM | 1770 | O | THR | A | 122 | 8.276 | 8.289 | 62.186 | 1.00 | 9.62 | O |
| ATOM | 1772 | N | ALA | A | 123 | 10.492 | 8.720 | 61.861 | 1.00 | 8.97 | N |
| ATOM | 1773 | CA | ALA | A | 123 | 10.395 | 8.461 | 60.406 | 1.00 | 8.00 | C |
| ATOM | 1775 | CB | ALA | A | 123 | 11.726 | 8.688 | 59.757 | 1.00 | 7.19 | C |
| ATOM | 1779 | C | ALA | A | 123 | 9.923 | 7.059 | 60.106 | 1.00 | 8.48 | C |
| ATOM | 1780 | O | ALA | A | 123 | 10.148 | 6.108 | 60.915 | 1.00 | 9.30 | O |
| ATOM | 1782 | N | THR | A | 124 | 9.256 | 6.923 | 58.946 | 1.00 | 9.17 | N |
| ATOM | 1783 | CA | THR | A | 124 | 8.735 | 5.656 | 58.427 | 1.00 | 8.89 | C |
| ATOM | 1785 | CB | THR | A | 124 | 7.192 | 5.636 | 58.489 | 1.00 | 9.66 | C |
| ATOM | 1787 | OG1 | THR | A | 124 | 6.643 | 6.543 | 57.507 | 1.00 | 10.24 | O |
| ATOM | 1789 | CG2 | THR | A | 124 | 6.696 | 5.997 | 59.853 | 1.00 | 9.70 | C |
| ATOM | 1793 | C | THR | A | 124 | 9.171 | 5.492 | 56.967 | 1.00 | 9.28 | C |
| ATOM | 1794 | O | THR | A | 124 | 9.957 | 6.280 | 56.448 | 1.00 | 8.87 | O |
| ATOM | 1796 | N | ALA | A | 125 | 8.645 | 4.469 | 56.296 | 1.00 | 8.98 | N |
| ATOM | 1797 | CA | ALA | A | 125 | 9.009 | 4.281 | 54.880 | 1.00 | 8.70 | C |
| ATOM | 1799 | CB | ALA | A | 125 | 8.402 | 3.030 | 54.361 | 1.00 | 9.55 | C |
| ATOM | 1803 | C | ALA | A | 125 | 8.537 | 5.463 | 54.028 | 1.00 | 8.90 | C |
| ATOM | 1804 | O | ALA | A | 125 | 9.105 | 5.729 | 52.971 | 1.00 | 8.92 | O |
| ATOM | 1806 | N | LEU | A | 126 | 7.476 | 6.146 | 54.487 | 1.00 | 8.75 | N |
| ATOM | 1807 | CA | LEU | A | 126 | 6.824 | 7.189 | 53.684 | 1.00 | 8.77 | C |
| ATOM | 1809 | CB | LEU | A | 126 | 5.325 | 6.855 | 53.591 | 1.00 | 9.83 | C |
| ATOM | 1812 | CG | LEU | A | 126 | 4.982 | 5.534 | 52.898 | 1.00 | 12.28 | C |
| ATOM | 1814 | CD1 | LEU | A | 126 | 3.476 | 5.318 | 52.909 | 1.00 | 13.65 | C |
| ATOM | 1818 | CD2 | LEU | A | 126 | 5.463 | 5.531 | 51.482 | 1.00 | 13.01 | C |
| ATOM | 1822 | C | LEU | A | 126 | 6.924 | 8.590 | 54.227 | 1.00 | 9.15 | C |
| ATOM | 1823 | O | LEU | A | 126 | 6.493 | 9.552 | 53.551 | 1.00 | 8.84 | O |
| ATOM | 1825 | N | THR | A | 127 | 7.436 | 8.732 | 55.448 | 1.00 | 8.64 | N |
| ATOM | 1826 | CA | THR | A | 127 | 7.471 | 10.039 | 56.115 | 1.00 | 8.59 | C |
| ATOM | 1828 | CB | THR | A | 127 | 6.337 | 10.202 | 57.149 | 1.00 | 9.13 | C |
| ATOM | 1830 | OG1 | THR | A | 127 | 6.472 | 9.200 | 58.181 | 1.00 | 10.34 | O |
| ATOM | 1832 | CG2 | THR | A | 127 | 4.938 | 10.113 | 56.529 | 1.00 | 9.97 | C |
| ATOM | 1836 | C | THR | A | 127 | 8.798 | 10.216 | 56.857 | 1.00 | 8.79 | C |
| ATOM | 1837 | O | THR | A | 127 | 9.374 | 9.219 | 57.290 | 1.00 | 9.36 | O |
| ATOM | 1839 | N | PRO | A | 128 | 9.273 | 11.464 | 57.056 | 1.00 | 7.86 | N |
| ATOM | 1840 | CA | PRO | A | 128 | 8.749 | 12.723 | 56.502 | 1.00 | 8.31 | C |
| ATOM | 1842 | CB | PRO | A | 128 | 9.756 | 13.757 | 56.897 | 1.00 | 8.97 | C |
| ATOM | 1845 | CG | PRO | A | 128 | 10.676 | 13.119 | 57.920 | 1.00 | 11.44 | C |
| ATOM | 1848 | CD | PRO | A | 128 | 10.521 | 11.642 | 57.837 | 1.00 | 8.52 | C |
| ATOM | 1851 | C | PRO | A | 128 | 8.595 | 12.637 | 54.982 | 1.00 | 8.34 | C |
| ATOM | 1852 | O | PRO | A | 128 | 9.390 | 11.977 | 54.321 | 1.00 | 8.32 | O |
| ATOM | 1853 | N | ALA | A | 129 | 7.521 | 13.256 | 54.480 | 1.00 | 7.80 | N |
| ATOM | 1854 | CA | ALA | A | 129 | 7.226 | 13.185 | 53.048 | 1.00 | 7.67 | C |
| ATOM | 1856 | CB | ALA | A | 129 | 6.063 | 14.115 | 52.674 | 1.00 | 7.71 | C |
| ATOM | 1860 | C | ALA | A | 129 | 8.476 | 13.501 | 52.240 | 1.00 | 7.65 | C |
| ATOM | 1861 | O | ALA | A | 129 | 9.249 | 14.425 | 52.549 | 1.00 | 7.64 | O |
| ATOM | 1863 | N | TRP | A | 130 | 8.683 | 12.696 | 51.201 | 1.00 | 7.53 | N |
| ATOM | 1864 | CA | TRP | A | 130 | 9.896 | 12.673 | 50.390 | 1.00 | 7.75 | C |
| ATOM | 1866 | CB | TRP | A | 130 | 10.025 | 11.339 | 49.697 | 1.00 | 8.17 | C |
| ATOM | 1869 | CG | TRP | A | 130 | 10.217 | 10.140 | 50.611 | 1.00 | 7.44 | C |
| ATOM | 1870 | CD1 | TRP | A | 130 | 9.246 | 9.260 | 51.022 | 1.00 | 8.39 | C |
| ATOM | 1872 | NE1 | TRP | A | 130 | 9.803 | 8.288 | 51.843 | 1.00 | 8.80 | N |
| ATOM | 1874 | CE2 | TRP | A | 130 | 11.158 | 8.509 | 51.940 | 1.00 | 7.48 | C |
| ATOM | 1875 | CD2 | TRP | A | 130 | 11.456 | 9.690 | 51.220 | 1.00 | 7.51 | C |
| ATOM | 1876 | CE3 | TRP | A | 130 | 12.767 | 10.137 | 51.175 | 1.00 | 9.24 | C |
| ATOM | 1878 | CZ3 | TRP | A | 130 | 13.729 | 9.425 | 51.829 | 1.00 | 8.16 | C |
| ATOM | 1880 | CH2 | TRP | A | 130 | 13.415 | 8.274 | 52.564 | 1.00 | 9.62 | C |
| ATOM | 1882 | CZ2 | TRP | A | 130 | 12.139 | 7.800 | 52.640 | 1.00 | 8.22 | C |
| ATOM | 1884 | C | TRP | A | 130 | 9.882 | 13.792 | 49.350 | 1.00 | 8.08 | C |
| ATOM | 1885 | O | TRP | A | 130 | 9.684 | 13.553 | 48.140 | 1.00 | 9.57 | O |
| ATOM | 1887 | N | MET | A | 131 | 10.071 | 15.011 | 49.847 | 1.00 | 7.97 | N |
| ATOM | 1888 | CA | MET | A | 131 | 9.981 | 16.190 | 49.016 | 1.00 | 8.31 | C |
| ATOM | 1890 | CB | MET | A | 131 | 8.509 | 16.422 | 48.584 | 1.00 | 9.63 | C |
| ATOM | 1893 | CG | MET | A | 131 | 7.585 | 16.674 | 49.801 | 1.00 | 8.68 | C |
| ATOM | 1896 | SD | MET | A | 131 | 5.828 | 16.800 | 49.393 | 1.00 | 8.53 | S |
| ATOM | 1897 | CE | MET | A | 131 | 5.459 | 15.151 | 48.784 | 1.00 | 8.19 | C |
| ATOM | 1901 | C | MET | A | 131 | 10.452 | 17.392 | 49.823 | 1.00 | 8.65 | C |
| ATOM | 1902 | O | MET | A | 131 | 10.399 | 17.415 | 51.058 | 1.00 | 8.31 | O |
| ATOM | 1904 | N | GLU | A | 132 | 10.926 | 18.409 | 49.102 | 1.00 | 8.97 | N |
| ATOM | 1905 | CA | GLU | A | 132 | 11.142 | 19.723 | 49.712 | 1.00 | 8.68 | C |
| ATOM | 1907 | CB | GLU | A | 132 | 11.613 | 20.750 | 48.659 | 1.00 | 9.38 | C |
| ATOM | 1910 | CG | GLU | A | 132 | 12.115 | 22.055 | 49.280 | 1.00 | 9.41 | C |
| ATOM | 1913 | CD | GLU | A | 132 | 12.825 | 22.977 | 48.300 | 1.00 | 12.57 | C |
| ATOM | 1914 | OE1 | GLU | A | 132 | 12.952 | 22.614 | 47.088 | 1.00 | 17.82 | O |
| ATOM | 1915 | OE2 | GLU | A | 132 | 13.203 | 24.090 | 48.751 | 1.00 | 14.38 | O |
| ATOM | 1916 | C | GLU | A | 132 | 9.816 | 20.160 | 50.334 | 1.00 | 9.39 | C |
| ATOM | 1917 | O | GLU | A | 132 | 8.784 | 20.126 | 49.641 | 1.00 | 9.43 | O |
| ATOM | 1919 | N | ASN | A | 133 | 9.876 | 20.595 | 51.595 | 1.00 | 9.49 | N |
| ATOM | 1920 | CA | ASN | A | 133 | 8.699 | 20.897 | 52.432 | 1.00 | 10.02 | C |
| ATOM | 1922 | CB | ASN | A | 133 | 7.832 | 21.978 | 51.781 | 1.00 | 11.85 | C |
| ATOM | 1925 | CG | ASN | A | 133 | 8.611 | 23.235 | 51.548 | 1.00 | 13.75 | C |
| ATOM | 1926 | OD1 | ASN | A | 133 | 9.246 | 23.732 | 52.480 | 1.00 | 19.98 | O |
| ATOM | 1927 | ND2 | ASN | A | 133 | 8.568 | 23.774 | 50.323 | 1.00 | 20.37 | N |
| ATOM | 1930 | C | ASN | A | 133 | 7.952 | 19.565 | 52.636 | 1.00 | 9.90 | C |
| ATOM | 1931 | O | ASN | A | 133 | 6.964 | 19.276 | 51.936 | 1.00 | 10.29 | O |
| ATOM | 1933 | N | PRO | A | 134 | 8.348 | 18.784 | 53.667 | 1.00 | 8.19 | N |
| ATOM | 1934 | CA | PRO | A | 134 | 9.081 | 19.193 | 54.857 | 1.00 | 8.07 | C |
| ATOM | 1936 | CB | PRO | A | 134 | 8.806 | 18.075 | 55.835 | 1.00 | 7.79 | C |
| ATOM | 1939 | CG | PRO | A | 134 | 8.589 | 16.870 | 54.980 | 1.00 | 8.16 | C |
| ATOM | 1942 | CD | PRO | A | 134 | 7.875 | 17.398 | 53.765 | 1.00 | 7.85 | C |
| ATOM | 1945 | C | PRO | A | 134 | 10.573 | 19.393 | 54.735 | 1.00 | 7.98 | C |
| ATOM | 1946 | O | PRO | A | 134 | 11.143 | 20.183 | 55.503 | 1.00 | 9.41 | O |
| ATOM | 1947 | N | TYR | A | 135 | 11.226 | 18.626 | 53.867 | 1.00 | 7.28 | N |
| ATOM | 1948 | CA | TYR | A | 135 | 12.685 | 18.708 | 53.827 | 1.00 | 8.17 | C |
| ATOM | 1950 | CB | TYR | A | 135 | 13.272 | 17.691 | 52.860 | 1.00 | 7.94 | C |
| ATOM | 1953 | CG | TYR | A | 135 | 13.344 | 16.303 | 53.449 | 1.00 | 7.81 | C |
| ATOM | 1954 | CD1 | TYR | A | 135 | 14.400 | 15.969 | 54.302 | 1.00 | 7.46 | C |
| ATOM | 1956 | CE1 | TYR | A | 135 | 14.494 | 14.725 | 54.880 | 1.00 | 7.44 | C |
| ATOM | 1958 | CZ | TYR | A | 135 | 13.546 | 13.749 | 54.651 | 1.00 | 10.60 | C |
| ATOM | 1959 | OH | TYR | A | 135 | 13.623 | 12.511 | 55.280 | 1.00 | 11.23 | O |
| ATOM | 1961 | CE2 | TYR | A | 135 | 12.467 | 14.038 | 53.802 | 1.00 | 9.66 | C |
| ATOM | 1963 | CD2 | TYR | A | 135 | 12.382 | 15.327 | 53.205 | 1.00 | 8.82 | C |
| ATOM | 1965 | C | TYR | A | 135 | 13.147 | 20.120 | 53.469 | 1.00 | 8.11 | C |
| ATOM | 1966 | O | TYR | A | 135 | 12.553 | 20.824 | 52.626 | 1.00 | 8.60 | O |
| ATOM | 1968 | N | ILE | A | 136 | 14.224 | 20.520 | 54.144 | 1.00 | 8.41 | N |
| ATOM | 1969 | CA | ILE | A | 136 | 14.860 | 21.822 | 53.934 | 1.00 | 8.73 | C |
| ATOM | 1971 | CB | ILE | A | 136 | 15.363 | 22.435 | 55.274 | 1.00 | 9.60 | C |
| ATOM | 1973 | CG1 | ILE | A | 136 | 14.201 | 22.577 | 56.267 | 1.00 | 11.04 | C |
| ATOM | 1976 | CD1 | ILE | A | 136 | 14.587 | 22.844 | 57.736 | 1.00 | 13.67 | C |
| ATOM | 1980 | CG2 | ILE | A | 136 | 16.095 | 23.749 | 54.978 | 1.00 | 10.67 | C |
| ATOM | 1984 | C | ILE | A | 136 | 16.032 | 21.629 | 52.965 | 1.00 | 9.36 | C |
| ATOM | 1985 | O | ILE | A | 136 | 16.973 | 20.828 | 53.217 | 1.00 | 8.57 | O |
| ATOM | 1987 | N | LYS | A | 137 | 15.979 | 22.326 | 51.833 | 1.00 | 9.57 | N |
| ATOM | 1988 | CA | LYS | A | 137 | 17.011 | 22.190 | 50.828 | 1.00 | 10.16 | C |
| ATOM | 1990 | CB | LYS | A | 137 | 16.547 | 22.722 | 49.479 | 1.00 | 10.97 | C |
| ATOM | 1993 | CG | LYS | A | 137 | 17.639 | 22.695 | 48.426 | 1.00 | 12.27 | C |
| ATOM | 1996 | CD | LYS | A | 137 | 17.156 | 23.116 | 47.044 | 1.00 | 14.11 | C |
| ATOM | 1999 | CE | LYS | A | 137 | 18.242 | 23.092 | 46.049 | 1.00 | 17.64 | C |
| ATOM | 2002 | NZ | LYS | A | 137 | 17.759 | 23.572 | 44.737 | 1.00 | 21.53 | N |
| ATOM | 2006 | C | LYS | A | 137 | 18.274 | 22.909 | 51.282 | 1.00 | 10.14 | C |
| ATOM | 2007 | O | LYS | A | 137 | 18.209 | 24.117 | 51.631 | 1.00 | 11.61 | O |
| ATOM | 2009 | N | VAL | A | 138 | 19.391 | 22.177 | 51.323 | 1.00 | 10.00 | N |
| ATOM | 2010 | CA | VAL | A | 138 | 20.662 | 22.750 | 51.744 | 1.00 | 9.75 | C |
| ATOM | 2012 | CB | VAL | A | 138 | 21.508 | 21.766 | 52.568 | 1.00 | 10.72 | C |
| ATOM | 2014 | CG1 | VAL | A | 138 | 22.809 | 22.438 | 53.017 | 1.00 | 11.63 | C |
| ATOM | 2018 | CG2 | VAL | A | 138 | 20.746 | 21.229 | 53.746 | 1.00 | 11.62 | C |
| ATOM | 2022 | C | VAL | A | 138 | 21.428 | 23.222 | 50.523 | 1.00 | 10.39 | C |
| ATOM | 2023 | O | VAL | A | 138 | 21.892 | 24.373 | 50.521 | 1.00 | 11.53 | O |
| ATOM | 2025 | N | ASP | A | 139 | 21.580 | 22.355 | 49.516 | 1.00 | 10.55 | N |
| ATOM | 2026 | CA | ASP | A | 139 | 22.384 | 22.691 | 48.314 | 1.00 | 9.26 | C |
| ATOM | 2028 | CB | ASP | A | 139 | 23.874 | 22.700 | 48.644 | 1.00 | 9.42 | C |
| ATOM | 2031 | CG | ASP | A | 139 | 24.738 | 23.339 | 47.558 | 1.00 | 12.48 | C |
| ATOM | 2032 | OD1 | ASP | A | 139 | 24.306 | 24.316 | 46.912 | 1.00 | 15.60 | O |
| ATOM | 2033 | OD2 | ASP | A | 139 | 25.895 | 22.865 | 47.416 | 1.00 | 13.71 | O |
| ATOM | 2034 | C | ASP | A | 139 | 22.166 | 21.658 | 47.252 | 1.00 | 8.41 | C |
| ATOM | 2035 | O | ASP | A | 139 | 22.045 | 20.489 | 47.573 | 1.00 | 9.94 | O |
| ATOM | 2037 | N | THR | A | 140 | 22.108 | 22.090 | 46.003 | 1.00 | 9.42 | N |
| ATOM | 2038 | CA | THR | A | 140 | 22.301 | 21.170 | 44.856 | 1.00 | 9.48 | C |
| ATOM | 2040 | CB | THR | A | 140 | 21.510 | 21.576 | 43.591 | 1.00 | 10.64 | C |
| ATOM | 2042 | OG1 | THR | A | 140 | 20.096 | 21.488 | 43.845 | 1.00 | 12.64 | O |
| ATOM | 2044 | CG2 | THR | A | 140 | 21.857 | 20.638 | 42.464 | 1.00 | 11.25 | C |
| ATOM | 2048 | C | THR | A | 140 | 23.809 | 21.192 | 44.615 | 1.00 | 10.29 | C |
| ATOM | 2049 | O | THR | A | 140 | 24.403 | 22.247 | 44.234 | 1.00 | 11.10 | O |
| ATOM | 2051 | N | VAL | A | 141 | 24.423 | 20.055 | 44.914 | 1.00 | 9.97 | N |
| ATOM | 2052 | CA | VAL | A | 141 | 25.890 | 19.898 | 44.924 | 1.00 | 10.16 | C |
| ATOM | 2054 | CB | VAL | A | 141 | 26.277 | 18.801 | 45.938 | 1.00 | 10.62 | C |
| ATOM | 2056 | CG1 | VAL | A | 141 | 27.799 | 18.563 | 45.949 | 1.00 | 10.86 | C |
| ATOM | 2060 | CG2 | VAL | A | 141 | 25.834 | 19.189 | 47.341 | 1.00 | 10.37 | C |
| ATOM | 2064 | C | VAL | A | 141 | 26.408 | 19.547 | 43.546 | 1.00 | 11.24 | C |
| ATOM | 2065 | O | VAL | A | 141 | 25.976 | 18.541 | 42.963 | 1.00 | 10.48 | O |
| ATOM | 2067 | N | ALA | A | 142 | 27.352 | 20.360 | 43.056 | 1.00 | 11.87 | N |
| ATOM | 2068 | CA | ALA | A | 142 | 27.983 | 20.108 | 41.768 | 1.00 | 13.96 | C |
| ATOM | 2070 | CB | ALA | A | 142 | 28.145 | 21.383 | 41.002 | 1.00 | 14.94 | C |
| ATOM | 2074 | C | ALA | A | 142 | 29.318 | 19.393 | 41.992 | 1.00 | 14.04 | C |
| ATOM | 2075 | O | ALA | A | 142 | 29.862 | 19.299 | 43.121 | 1.00 | 14.70 | O |
| ATOM | 2077 | N | ALA | A | 143 | 29.803 | 18.761 | 40.939 | 1.00 | 13.39 | N |
| ATOM | 2078 | CA | ALA | A | 143 | 31.028 | 18.031 | 41.014 | 1.00 | 12.05 | C |
| ATOM | 2080 | CB | ALA | A | 143 | 30.843 | 16.605 | 40.498 | 1.00 | 12.80 | C |
| ATOM | 2084 | C | ALA | A | 143 | 32.084 | 18.743 | 40.182 | 1.00 | 12.33 | C |
| ATOM | 2085 | O | ALA | A | 143 | 31.951 | 18.796 | 38.937 | 1.00 | 14.44 | O |
| ATOM | 2087 | N | GLU | A | 144 | 33.139 | 19.250 | 40.825 | 1.00 | 13.57 | N |
| ATOM | 2088 | CA | GLU | A | 144 | 34.280 | 19.809 | 40.083 | 1.00 | 13.75 | C |
| ATOM | 2090 | CB | GLU | A | 144 | 35.250 | 20.550 | 41.014 | 1.00 | 16.16 | C |
| ATOM | 2097 | C | GLU | A | 144 | 35.085 | 18.722 | 39.347 | 1.00 | 12.44 | C |
| ATOM | 2098 | O | GLU | A | 144 | 35.769 | 18.992 | 38.333 | 1.00 | 14.14 | O |
| ATOM | 2100 | N | HIS | A | 145 | 35.027 | 17.493 | 39.860 | 1.00 | 10.12 | N |
| ATOM | 2101 | CA | HIS | A | 145 | 35.796 | 16.368 | 39.332 | 1.00 | 10.38 | C |
| ATOM | 2103 | CB | HIS | A | 145 | 36.700 | 15.784 | 40.422 | 1.00 | 11.19 | C |
| ATOM | 2106 | CG | HIS | A | 145 | 37.540 | 16.818 | 41.088 | 1.00 | 14.12 | C |
| ATOM | 2107 | ND1 | HIS | A | 145 | 38.612 | 17.412 | 40.461 | 1.00 | 15.31 | N |
| ATOM | 2109 | CE1 | HIS | A | 145 | 39.149 | 18.314 | 41.270 | 1.00 | 15.39 | C |
| ATOM | 2111 | NE2 | HIS | A | 145 | 38.484 | 18.302 | 42.410 | 1.00 | 15.57 | N |
| ATOM | 2113 | CD2 | HIS | A | 145 | 37.464 | 17.374 | 42.319 | 1.00 | 15.78 | C |
| ATOM | 2115 | C | HIS | A | 145 | 34.901 | 15.279 | 38.826 | 1.00 | 9.44 | C |
| ATOM | 2116 | O | HIS | A | 145 | 34.166 | 14.653 | 39.601 | 1.00 | 9.58 | O |
| ATOM | 2118 | N | LEU | A | 146 | 34.918 | 15.065 | 37.515 | 1.00 | 8.62 | N |
| ATOM | 2119 | CA | LEU | A | 146 | 34.147 | 13.985 | 36.905 | 1.00 | 8.83 | C |
| ATOM | 2121 | CB | LEU | A | 146 | 33.532 | 14.458 | 35.583 | 1.00 | 7.80 | C |
| ATOM | 2124 | CG | LEU | A | 146 | 32.613 | 15.647 | 35.720 | 1.00 | 10.46 | C |
| ATOM | 2126 | CD1 | LEU | A | 146 | 32.213 | 16.186 | 34.352 | 1.00 | 13.82 | C |
| ATOM | 2130 | CD2 | LEU | A | 146 | 31.360 | 15.319 | 36.532 | 1.00 | 12.33 | C |
| ATOM | 2134 | C | LEU | A | 146 | 35.039 | 12.782 | 36.641 | 1.00 | 8.88 | C |
| ATOM | 2135 | O | LEU | A | 146 | 36.196 | 12.917 | 36.142 | 1.00 | 10.66 | O |
| ATOM | 2137 | N | THR | A | 147 | 34.519 | 11.603 | 36.989 | 1.00 | 7.96 | N |
| ATOM | 2138 | CA | THR | A | 147 | 35.160 | 10.340 | 36.676 | 1.00 | 8.03 | C |
| ATOM | 2140 | CB | THR | A | 147 | 34.756 | 9.277 | 37.693 | 1.00 | 9.05 | C |
| ATOM | 2142 | OG1 | THR | A | 147 | 35.293 | 9.664 | 38.977 | 1.00 | 9.24 | O |
| ATOM | 2144 | CG2 | THR | A | 147 | 35.234 | 7.926 | 37.337 | 1.00 | 9.73 | C |
| ATOM | 2148 | C | THR | A | 147 | 34.826 | 9.923 | 35.257 | 1.00 | 8.52 | C |
| ATOM | 2149 | O | THR | A | 147 | 33.661 | 10.037 | 34.830 | 1.00 | 8.12 | O |
| ATOM | 2151 | N | ARG | A | 148 | 35.858 | 9.490 | 34.542 | 1.00 | 7.65 | N |
| ATOM | 2152 | CA | ARG | A | 148 | 35.756 | 9.129 | 33.116 | 1.00 | 9.81 | C |
| ATOM | 2154 | CB | ARG | A | 148 | 36.751 | 9.921 | 32.277 | 1.00 | 10.51 | C |
| ATOM | 2157 | CG | ARG | A | 148 | 36.501 | 11.417 | 32.300 | 1.00 | 11.89 | C |
| ATOM | 2160 | CD | ARG | A | 148 | 37.608 | 12.217 | 31.636 | 1.00 | 13.56 | C |
| ATOM | 2163 | NE | ARG | A | 148 | 37.761 | 11.745 | 30.264 | 1.00 | 18.52 | N |
| ATOM | 2165 | CZ | ARG | A | 148 | 37.240 | 12.319 | 29.175 | 1.00 | 17.48 | C |
| ATOM | 2166 | NH1 | ARG | A | 148 | 36.501 | 13.464 | 29.244 | 1.00 | 12.11 | N |
| ATOM | 2169 | NH2 | ARG | A | 148 | 37.438 | 11.695 | 27.998 | 1.00 | 16.48 | N |
| ATOM | 2172 | C | ARG | A | 148 | 36.026 | 7.631 | 32.940 | 1.00 | 10.90 | C |
| ATOM | 2173 | O | ARG | A | 148 | 36.801 | 7.072 | 33.695 | 1.00 | 10.86 | O |
| ATOM | 2175 | N | LYS | A | 149 | 35.376 | 7.005 | 31.962 | 1.00 | 11.83 | N |
| ATOM | 2176 | CA | LYS | A | 149 | 35.681 | 5.589 | 31.597 | 1.00 | 14.09 | C |
| ATOM | 2178 | CB | LYS | A | 149 | 34.911 | 4.565 | 32.415 | 1.00 | 15.18 | C |
| ATOM | 2181 | CG | LYS | A | 149 | 35.164 | 3.097 | 31.939 | 1.00 | 16.23 | C |
| ATOM | 2184 | CD | LYS | A | 149 | 34.683 | 2.035 | 32.871 | 1.00 | 19.77 | C |
| ATOM | 2187 | CE | LYS | A | 149 | 35.136 | 0.618 | 32.360 | 1.00 | 17.69 | C |
| ATOM | 2190 | NZ | LYS | A | 149 | 35.284 | 0.467 | 30.843 | 1.00 | 22.60 | N |
| ATOM | 2194 | C | LYS | A | 149 | 35.449 | 5.411 | 30.116 | 1.00 | 16.14 | C |
| ATOM | 2195 | O | LYS | A | 149 | 34.402 | 5.773 | 29.600 | 1.00 | 16.09 | O |
| ATOM | 2197 | N | ARG | A | 150 | 36.445 | 4.841 | 29.449 | 1.00 | 16.85 | N |
| ATOM | 2198 | CA | ARG | A | 150 | 36.590 | 4.806 | 27.965 | 1.00 | 17.08 | C |
| ATOM | 2200 | CB | ARG | A | 150 | 37.942 | 5.349 | 27.720 | 1.00 | 19.68 | C |
| ATOM | 2203 | CG | ARG | A | 150 | 38.557 | 4.959 | 26.571 | 1.00 | 19.86 | C |
| ATOM | 2206 | CD | ARG | A | 150 | 37.760 | 5.417 | 25.529 | 1.00 | 22.77 | C |
| ATOM | 2209 | NE | ARG | A | 150 | 37.895 | 6.784 | 25.135 | 1.00 | 23.77 | N |
| ATOM | 2211 | CZ | ARG | A | 150 | 38.499 | 7.765 | 25.788 | 1.00 | 20.28 | C |
| ATOM | 2212 | NH1 | ARG | A | 150 | 38.461 | 8.938 | 25.172 | 1.00 | 22.53 | N |
| ATOM | 2215 | NH2 | ARG | A | 150 | 39.131 | 7.624 | 26.975 | 1.00 | 18.40 | N |
| ATOM | 2218 | C | ARG | A | 150 | 36.544 | 3.358 | 27.660 | 1.00 | 16.33 | C |
| ATOM | 2219 | O | ARG | A | 150 | 37.001 | 2.613 | 28.491 | 1.00 | 16.88 | O |
| ATOM | 2221 | N | PRO | A | 151 | 35.945 | 2.927 | 26.503 | 1.00 | 15.43 | N |
| ATOM | 2222 | CA | PRO | A | 151 | 35.669 | 1.481 | 26.330 | 1.00 | 14.71 | C |
| ATOM | 2224 | CB | PRO | A | 151 | 35.041 | 1.386 | 24.925 | 1.00 | 15.62 | C |
| ATOM | 2227 | CG | PRO | A | 151 | 35.376 | 2.710 | 24.211 | 1.00 | 15.64 | C |
| ATOM | 2230 | CD | PRO | A | 151 | 35.407 | 3.704 | 25.367 | 1.00 | 16.12 | C |
| ATOM | 2233 | C | PRO | A | 151 | 36.926 | 0.644 | 26.427 | 1.00 | 14.70 | C |
| ATOM | 2234 | O | PRO | A | 151 | 37.895 | 1.013 | 25.751 | 1.00 | 10.80 | O |
| ATOM | 2235 | N | GLY | A | 152 | 36.885 | −0.437 | 27.224 | 1.00 | 11.37 | N |
| ATOM | 2236 | CA | GLY | A | 152 | 38.053 | −1.334 | 27.452 | 1.00 | 13.67 | C |
| ATOM | 2239 | C | GLY | A | 152 | 38.962 | −1.104 | 28.672 | 1.00 | 13.66 | C |
| ATOM | 2240 | O | GLY | A | 152 | 39.807 | −1.950 | 29.084 | 1.00 | 12.78 | O |
| ATOM | 2242 | N | ALA | A | 153 | 38.745 | 0.033 | 29.337 | 1.00 | 13.91 | N |
| ATOM | 2243 | CA | ALA | A | 153 | 39.737 | 0.621 | 30.227 | 1.00 | 12.51 | C |
| ATOM | 2245 | CB | ALA | A | 153 | 40.168 | 1.917 | 29.612 | 1.00 | 11.34 | C |
| ATOM | 2249 | C | ALA | A | 153 | 39.258 | 0.882 | 31.660 | 1.00 | 12.62 | C |
| ATOM | 2250 | O | ALA | A | 153 | 38.043 | 1.004 | 31.877 | 1.00 | 14.96 | O |
| ATOM | 2252 | N | GLU | A | 154 | 40.182 | 0.992 | 32.614 | 1.00 | 12.32 | N |
| ATOM | 2253 | CA | GLU | A | 154 | 39.825 | 1.383 | 33.966 | 1.00 | 12.28 | C |
| ATOM | 2255 | CB | GLU | A | 154 | 40.977 | 1.233 | 34.934 | 1.00 | 13.61 | C |
| ATOM | 2262 | C | GLU | A | 154 | 39.329 | 2.818 | 34.003 | 1.00 | 11.59 | C |
| ATOM | 2263 | O | GLU | A | 154 | 39.850 | 3.677 | 33.311 | 1.00 | 9.51 | O |
| ATOM | 2265 | N | ALA | A | 155 | 38.349 | 3.064 | 34.856 | 1.00 | 12.64 | N |
| ATOM | 2266 | CA | ALA | A | 155 | 37.901 | 4.424 | 35.135 | 1.00 | 11.59 | C |
| ATOM | 2268 | CB | ALA | A | 155 | 36.676 | 4.385 | 36.073 | 1.00 | 12.13 | C |
| ATOM | 2272 | C | ALA | A | 155 | 39.026 | 5.256 | 35.750 | 1.00 | 11.54 | C |
| ATOM | 2273 | O | ALA | A | 155 | 39.922 | 4.759 | 36.495 | 1.00 | 12.39 | O |
| ATOM | 2275 | N | THR | A | 156 | 39.024 | 6.567 | 35.451 | 1.00 | 10.29 | N |
| ATOM | 2276 | CA | THR | A | 156 | 39.991 | 7.487 | 36.018 | 1.00 | 10.85 | C |
| ATOM | 2278 | CB | THR | A | 156 | 41.025 | 8.000 | 34.960 | 1.00 | 12.01 | C |
| ATOM | 2280 | OG1 | THR | A | 156 | 40.344 | 8.740 | 33.942 | 1.00 | 16.35 | O |
| ATOM | 2282 | CG2 | THR | A | 156 | 41.787 | 6.861 | 34.355 | 1.00 | 14.57 | C |
| ATOM | 2286 | C | THR | A | 156 | 39.242 | 8.676 | 36.572 | 1.00 | 11.26 | C |
| ATOM | 2287 | O | THR | A | 156 | 38.272 | 9.105 | 35.972 | 1.00 | 11.47 | O |
| ATOM | 2289 | N | GLY | A | 157 | 39.679 | 9.210 | 37.699 | 1.00 | 11.15 | N |
| ATOM | 2290 | CA | GLY | A | 157 | 38.910 | 10.275 | 38.306 | 1.00 | 11.21 | C |
| ATOM | 2293 | C | GLY | A | 157 | 39.438 | 10.644 | 39.671 | 1.00 | 11.02 | C |
| ATOM | 2294 | O | GLY | A | 157 | 40.298 | 9.944 | 40.220 | 1.00 | 13.12 | O |
| ATOM | 2296 | N | LYS | A | 158 | 38.941 | 11.766 | 40.139 | 1.00 | 10.68 | N |
| ATOM | 2297 | CA | LYS | A | 158 | 39.181 | 12.210 | 41.524 | 1.00 | 9.49 | C |
| ATOM | 2299 | CB | LYS | A | 158 | 39.732 | 13.623 | 41.490 | 1.00 | 9.39 | C |
| ATOM | 2302 | CG | LYS | A | 158 | 41.165 | 13.767 | 40.912 | 1.00 | 10.25 | C |
| ATOM | 2305 | CD | LYS | A | 158 | 41.557 | 15.195 | 40.711 | 1.00 | 11.46 | C |
| ATOM | 2308 | CE | LYS | A | 158 | 43.011 | 15.314 | 40.210 | 1.00 | 15.64 | C |
| ATOM | 2311 | NZ | LYS | A | 158 | 43.357 | 16.712 | 39.708 | 1.00 | 16.95 | N |
| ATOM | 2315 | C | LYS | A | 158 | 37.913 | 12.200 | 42.330 | 1.00 | 9.39 | C |
| ATOM | 2316 | O | LYS | A | 158 | 36.804 | 12.363 | 41.805 | 1.00 | 9.89 | O |
| ATOM | 2318 | N | VAL | A | 159 | 38.098 | 12.081 | 43.627 | 1.00 | 9.10 | N |
| ATOM | 2319 | CA | VAL | A | 159 | 36.990 | 12.188 | 44.564 | 1.00 | 9.25 | C |
| ATOM | 2321 | CB | VAL | A | 159 | 37.323 | 11.430 | 45.876 | 1.00 | 10.17 | C |
| ATOM | 2323 | CG1 | VAL | A | 159 | 36.211 | 11.643 | 46.920 | 1.00 | 11.49 | C |
| ATOM | 2327 | CG2 | VAL | A | 159 | 37.535 | 9.939 | 45.589 | 1.00 | 11.58 | C |
| ATOM | 2331 | C | VAL | A | 159 | 36.674 | 13.646 | 44.850 | 1.00 | 9.07 | C |
| ATOM | 2332 | O | VAL | A | 159 | 37.570 | 14.466 | 44.949 | 1.00 | 10.34 | O |
| ATOM | 2334 | N | ASN | A | 160 | 35.375 | 13.967 | 44.962 | 1.00 | 8.44 | N |
| ATOM | 2335 | CA | ASN | A | 160 | 34.893 | 15.296 | 45.314 | 1.00 | 7.91 | C |
| ATOM | 2337 | CB | ASN | A | 160 | 33.565 | 15.595 | 44.602 | 1.00 | 8.74 | C |
| ATOM | 2340 | CG | ASN | A | 160 | 33.729 | 15.628 | 43.113 | 1.00 | 8.92 | C |
| ATOM | 2341 | OD1 | ASN | A | 160 | 34.114 | 16.647 | 42.578 | 1.00 | 9.80 | O |
| ATOM | 2342 | ND2 | ASN | A | 160 | 33.475 | 14.502 | 42.443 | 1.00 | 10.16 | N |
| ATOM | 2345 | C | ASN | A | 160 | 34.614 | 15.359 | 46.825 | 1.00 | 8.46 | C |
| ATOM | 2346 | O | ASN | A | 160 | 34.234 | 14.366 | 47.435 | 1.00 | 9.78 | O |
| ATOM | 2348 | N | VAL | A | 161 | 34.776 | 16.542 | 47.378 | 1.00 | 8.68 | N |
| ATOM | 2349 | CA | VAL | A | 161 | 34.398 | 16.796 | 48.777 | 1.00 | 8.98 | C |
| ATOM | 2351 | CB | VAL | A | 161 | 35.614 | 16.836 | 49.747 | 1.00 | 10.70 | C |
| ATOM | 2353 | CG1 | VAL | A | 161 | 36.624 | 17.942 | 49.409 | 1.00 | 11.76 | C |
| ATOM | 2357 | CG2 | VAL | A | 161 | 35.138 | 16.966 | 51.192 | 1.00 | 9.80 | C |
| ATOM | 2361 | C | VAL | A | 161 | 33.615 | 18.117 | 48.836 | 1.00 | 9.38 | C |
| ATOM | 2362 | O | VAL | A | 161 | 33.955 | 19.116 | 48.208 | 1.00 | 10.78 | O |
| ATOM | 2364 | N | LYS | A | 162 | 32.522 | 18.096 | 49.594 | 1.00 | 9.73 | N |
| ATOM | 2365 | CA | LYS | A | 162 | 31.708 | 19.264 | 49.873 | 1.00 | 9.18 | C |
| ATOM | 2367 | CB | LYS | A | 162 | 30.374 | 19.207 | 49.057 | 1.00 | 10.14 | C |
| ATOM | 2370 | CG | LYS | A | 162 | 29.491 | 20.452 | 49.235 | 1.00 | 13.01 | C |
| ATOM | 2373 | CD | LYS | A | 162 | 30.092 | 21.701 | 48.670 | 1.00 | 16.54 | C |
| ATOM | 2376 | CE | LYS | A | 162 | 29.144 | 22.929 | 48.785 | 1.00 | 19.66 | C |
| ATOM | 2379 | NZ | LYS | A | 162 | 29.808 | 24.238 | 48.416 | 1.00 | 22.42 | N |
| ATOM | 2383 | C | LYS | A | 162 | 31.361 | 19.219 | 51.374 | 1.00 | 9.98 | C |
| ATOM | 2384 | O | LYS | A | 162 | 30.879 | 18.163 | 51.860 | 1.00 | 10.27 | O |
| ATOM | 2386 | N | THR | A | 163 | 31.558 | 20.331 | 52.077 | 1.00 | 9.42 | N |
| ATOM | 2387 | CA | THR | A | 163 | 31.096 | 20.469 | 53.452 | 1.00 | 9.80 | C |
| ATOM | 2389 | CB | THR | A | 163 | 32.246 | 20.807 | 54.403 | 1.00 | 9.67 | C |
| ATOM | 2391 | OG1 | THR | A | 163 | 33.214 | 19.739 | 54.358 | 1.00 | 9.52 | O |
| ATOM | 2393 | CG2 | THR | A | 163 | 31.765 | 21.006 | 55.808 | 1.00 | 11.48 | C |
| ATOM | 2397 | C | THR | A | 163 | 29.992 | 21.530 | 53.491 | 1.00 | 9.97 | C |
| ATOM | 2398 | O | THR | A | 163 | 30.157 | 22.652 | 52.999 | 1.00 | 11.36 | O |
| ATOM | 2400 | N | LEU | A | 164 | 28.855 | 21.128 | 54.042 | 1.00 | 10.53 | N |
| ATOM | 2401 | CA | LEU | A | 164 | 27.671 | 21.932 | 54.209 | 1.00 | 10.36 | C |
| ATOM | 2403 | CB | LEU | A | 164 | 26.474 | 21.165 | 53.625 | 1.00 | 11.41 | C |
| ATOM | 2406 | CG | LEU | A | 164 | 26.646 | 20.822 | 52.143 | 1.00 | 13.33 | C |
| ATOM | 2408 | CD1 | LEU | A | 164 | 25.497 | 19.943 | 51.643 | 1.00 | 16.96 | C |
| ATOM | 2412 | CD2 | LEU | A | 164 | 26.756 | 22.072 | 51.298 | 1.00 | 16.24 | C |
| ATOM | 2416 | C | LEU | A | 164 | 27.440 | 22.268 | 55.670 | 1.00 | 10.61 | C |
| ATOM | 2417 | O | LEU | A | 164 | 27.750 | 21.473 | 56.555 | 1.00 | 12.15 | O |
| ATOM | 2419 | N | ARG | A | 165 | 26.914 | 23.463 | 55.924 | 1.00 | 10.93 | N |
| ATOM | 2420 | CA | ARG | A | 165 | 26.586 | 23.874 | 57.284 | 1.00 | 11.96 | C |
| ATOM | 2422 | CB | ARG | A | 165 | 27.171 | 25.240 | 57.599 | 1.00 | 12.49 | C |
| ATOM | 2425 | CG | ARG | A | 165 | 26.974 | 25.703 | 59.029 | 1.00 | 13.26 | C |
| ATOM | 2428 | CD | ARG | A | 165 | 27.616 | 27.089 | 59.136 | 1.00 | 15.90 | C |
| ATOM | 2431 | NE | ARG | A | 165 | 27.727 | 27.688 | 60.469 | 1.00 | 20.51 | N |
| ATOM | 2433 | CZ | ARG | A | 165 | 27.136 | 28.826 | 60.844 | 1.00 | 21.93 | C |
| ATOM | 2434 | NH1 | ARG | A | 165 | 26.352 | 29.490 | 60.013 | 1.00 | 23.30 | N |
| ATOM | 2437 | NH2 | ARG | A | 165 | 27.315 | 29.303 | 62.067 | 1.00 | 23.95 | N |
| ATOM | 2440 | C | ARG | A | 165 | 25.056 | 23.909 | 57.443 | 1.00 | 12.67 | C |
| ATOM | 2441 | O | ARG | A | 165 | 24.345 | 24.577 | 56.664 | 1.00 | 14.74 | O |
| ATOM | 2443 | N | LEU | A | 166 | 24.577 | 23.178 | 58.436 | 1.00 | 11.74 | N |
| ATOM | 2444 | CA | LEU | A | 166 | 23.132 | 22.964 | 58.690 | 1.00 | 11.51 | C |
| ATOM | 2446 | CB | LEU | A | 166 | 22.792 | 21.486 | 58.930 | 1.00 | 12.12 | C |
| ATOM | 2449 | CG | LEU | A | 166 | 23.316 | 20.409 | 58.006 | 1.00 | 17.08 | C |
| ATOM | 2451 | CD1 | LEU | A | 166 | 22.908 | 19.019 | 58.467 | 1.00 | 19.61 | C |
| ATOM | 2455 | CD2 | LEU | A | 166 | 22.886 | 20.648 | 56.641 | 1.00 | 20.41 | C |
| ATOM | 2459 | C | LEU | A | 166 | 22.748 | 23.732 | 59.945 | 1.00 | 11.98 | C |
| ATOM | 2460 | O | LEU | A | 166 | 23.450 | 23.645 | 60.930 | 1.00 | 12.34 | O |
| ATOM | 2462 | N | GLY | A | 167 | 21.563 | 24.386 | 59.937 | 1.00 | 12.12 | N |
| ATOM | 2463 | CA | GLY | A | 167 | 21.048 | 25.052 | 61.106 | 1.00 | 12.77 | C |
| ATOM | 2466 | C | GLY | A | 167 | 20.628 | 26.496 | 60.813 | 1.00 | 14.49 | C |
| ATOM | 2467 | O | GLY | A | 167 | 20.639 | 26.913 | 59.642 | 1.00 | 14.88 | O |
| ATOM | 2469 | N | PRO | A | 168 | 20.256 | 27.233 | 61.861 | 1.00 | 14.26 | N |
| ATOM | 2470 | CA | PRO | A | 168 | 20.320 | 26.798 | 63.252 | 1.00 | 13.89 | C |
| ATOM | 2472 | CB | PRO | A | 168 | 20.073 | 28.093 | 64.030 | 1.00 | 15.57 | C |
| ATOM | 2475 | CG | PRO | A | 168 | 19.159 | 28.871 | 63.098 | 1.00 | 16.62 | C |
| ATOM | 2478 | CD | PRO | A | 168 | 19.658 | 28.586 | 61.740 | 1.00 | 15.12 | C |
| ATOM | 2481 | C | PRO | A | 168 | 19.276 | 25.749 | 63.616 | 1.00 | 13.01 | C |
| ATOM | 2482 | O | PRO | A | 168 | 18.112 | 25.762 | 63.108 | 1.00 | 14.29 | O |
| ATOM | 2483 | N | LEU | A | 169 | 19.698 | 24.816 | 64.458 | 1.00 | 12.79 | N |
| ATOM | 2484 | CA | LEU | A | 169 | 18.906 | 23.655 | 64.842 | 1.00 | 11.32 | C |
| ATOM | 2486 | CB | LEU | A | 169 | 19.767 | 22.400 | 64.948 | 1.00 | 12.93 | C |
| ATOM | 2489 | CG | LEU | A | 169 | 20.561 | 22.078 | 63.691 | 1.00 | 13.60 | C |
| ATOM | 2491 | CD1 | LEU | A | 169 | 21.509 | 20.931 | 64.018 | 1.00 | 16.57 | C |
| ATOM | 2495 | CD2 | LEU | A | 169 | 19.626 | 21.728 | 62.550 | 1.00 | 15.53 | C |
| ATOM | 2499 | C | LEU | A | 169 | 18.244 | 23.882 | 66.193 | 1.00 | 13.18 | C |
| ATOM | 2500 | O | LEU | A | 169 | 18.778 | 24.595 | 67.052 | 1.00 | 13.33 | O |
| ATOM | 2502 | N | SER | A | 170 | 17.052 | 23.329 | 66.370 | 1.00 | 12.97 | N |
| ATOM | 2503 | CA | SER | A | 170 | 16.352 | 23.474 | 67.646 | 1.00 | 14.51 | C |
| ATOM | 2505 | CB | SER | A | 170 | 15.393 | 24.637 | 67.507 | 1.00 | 15.01 | C |
| ATOM | 2508 | OG | SER | A | 170 | 14.303 | 24.316 | 66.678 | 1.00 | 22.23 | O |
| ATOM | 2510 | C | SER | A | 170 | 15.600 | 22.251 | 68.173 | 1.00 | 13.02 | C |
| ATOM | 2511 | O | SER | A | 170 | 15.258 | 22.199 | 69.366 | 1.00 | 17.05 | O |
| ATOM | 2513 | N | LYS | A | 171 | 15.368 | 21.242 | 67.327 | 1.00 | 11.31 | N |
| ATOM | 2514 | CA | LYS | A | 171 | 14.722 | 20.011 | 67.784 | 1.00 | 10.60 | C |
| ATOM | 2516 | CB | LYS | A | 171 | 13.782 | 19.493 | 66.691 | 1.00 | 10.19 | C |
| ATOM | 2519 | CG | LYS | A | 171 | 12.594 | 20.391 | 66.388 | 1.00 | 11.75 | C |
| ATOM | 2522 | CD | LYS | A | 171 | 11.530 | 19.673 | 65.517 | 1.00 | 11.58 | C |
| ATOM | 2525 | CE | LYS | A | 171 | 10.212 | 20.409 | 65.393 | 1.00 | 14.91 | C |
| ATOM | 2528 | NZ | LYS | A | 171 | 9.250 | 19.814 | 64.374 | 1.00 | 15.60 | N |
| ATOM | 2532 | C | LYS | A | 171 | 15.732 | 18.959 | 68.255 | 1.00 | 10.82 | C |
| ATOM | 2533 | O | LYS | A | 171 | 16.956 | 19.151 | 68.092 | 1.00 | 12.33 | O |
| ATOM | 2535 | N | ALA | A | 172 | 15.274 | 17.855 | 68.823 | 1.00 | 9.49 | N |
| ATOM | 2536 | CA | ALA | A | 172 | 16.192 | 16.873 | 69.457 | 1.00 | 8.93 | C |
| ATOM | 2538 | CB | ALA | A | 172 | 15.405 | 15.776 | 70.239 | 1.00 | 9.36 | C |
| ATOM | 2542 | C | ALA | A | 172 | 17.180 | 16.212 | 68.488 | 1.00 | 9.88 | C |
| ATOM | 2543 | O | ALA | A | 172 | 18.304 | 15.868 | 68.911 | 1.00 | 11.09 | O |
| ATOM | 2545 | N | GLY | A | 173 | 16.756 | 16.017 | 67.245 | 1.00 | 9.34 | N |
| ATOM | 2546 | CA | GLY | A | 173 | 17.600 | 15.356 | 66.238 | 1.00 | 9.33 | C |
| ATOM | 2549 | C | GLY | A | 173 | 17.131 | 15.694 | 64.837 | 1.00 | 9.73 | C |
| ATOM | 2550 | O | GLY | A | 173 | 16.217 | 16.515 | 64.646 | 1.00 | 9.04 | O |
| ATOM | 2552 | N | PHE | A | 174 | 17.827 | 15.117 | 63.865 | 1.00 | 7.56 | N |
| ATOM | 2553 | CA | PHE | A | 174 | 17.552 | 15.372 | 62.466 | 1.00 | 7.53 | C |
| ATOM | 2555 | CB | PHE | A | 174 | 18.263 | 16.652 | 61.997 | 1.00 | 8.21 | C |
| ATOM | 2558 | CG | PHE | A | 174 | 19.749 | 16.581 | 62.118 | 1.00 | 8.24 | C |
| ATOM | 2559 | CD1 | PHE | A | 174 | 20.523 | 16.170 | 61.026 | 1.00 | 10.68 | C |
| ATOM | 2561 | CE1 | PHE | A | 174 | 21.904 | 16.075 | 61.163 | 1.00 | 11.00 | C |
| ATOM | 2563 | CZ | PHE | A | 174 | 22.494 | 16.378 | 62.367 | 1.00 | 9.97 | C |
| ATOM | 2565 | CE2 | PHE | A | 174 | 21.770 | 16.762 | 63.426 | 1.00 | 11.65 | C |
| ATOM | 2567 | CD2 | PHE | A | 174 | 20.374 | 16.875 | 63.317 | 1.00 | 11.58 | C |
| ATOM | 2569 | C | PHE | A | 174 | 17.936 | 14.203 | 61.550 | 1.00 | 8.39 | C |
| ATOM | 2570 | O | PHE | A | 174 | 18.735 | 13.329 | 61.906 | 1.00 | 9.32 | O |
| ATOM | 2572 | N | TYR | A | 175 | 17.336 | 14.228 | 60.359 | 1.00 | 8.25 | N |
| ATOM | 2573 | CA | TYR | A | 175 | 17.658 | 13.316 | 59.259 | 1.00 | 7.48 | C |
| ATOM | 2575 | CB | TYR | A | 175 | 16.413 | 12.637 | 58.676 | 1.00 | 9.30 | C |
| ATOM | 2578 | CG | TYR | A | 175 | 15.584 | 11.889 | 59.688 | 1.00 | 7.58 | C |
| ATOM | 2579 | CD1 | TYR | A | 175 | 15.835 | 10.546 | 59.969 | 1.00 | 8.86 | C |
| ATOM | 2581 | CE1 | TYR | A | 175 | 15.095 | 9.842 | 60.902 | 1.00 | 9.03 | C |
| ATOM | 2583 | CZ | TYR | A | 175 | 14.030 | 10.433 | 61.508 | 1.00 | 8.70 | C |
| ATOM | 2584 | OH | TYR | A | 175 | 13.278 | 9.774 | 62.464 | 1.00 | 8.58 | O |
| ATOM | 2586 | CE2 | TYR | A | 175 | 13.757 | 11.778 | 61.240 | 1.00 | 9.04 | C |
| ATOM | 2588 | CD2 | TYR | A | 175 | 14.519 | 12.480 | 60.322 | 1.00 | 9.76 | C |
| ATOM | 2590 | C | TYR | A | 175 | 18.321 | 14.102 | 58.154 | 1.00 | 9.02 | C |
| ATOM | 2591 | O | TYR | A | 175 | 17.914 | 15.248 | 57.902 | 1.00 | 9.13 | O |
| ATOM | 2593 | N | LEU | A | 176 | 19.326 | 13.515 | 57.485 | 1.00 | 9.33 | N |
| ATOM | 2594 | CA | LEU | A | 176 | 19.782 | 14.072 | 56.220 | 1.00 | 10.45 | C |
| ATOM | 2596 | CB | LEU | A | 176 | 21.319 | 14.087 | 56.124 | 1.00 | 11.50 | C |
| ATOM | 2599 | CG | LEU | A | 176 | 22.065 | 15.260 | 55.571 | 1.00 | 19.01 | C |
| ATOM | 2601 | CD1 | LEU | A | 176 | 21.729 | 16.554 | 56.106 | 1.00 | 24.20 | C |
| ATOM | 2605 | CD2 | LEU | A | 176 | 23.588 | 15.037 | 55.662 | 1.00 | 16.96 | C |
| ATOM | 2609 | C | LEU | A | 176 | 19.192 | 13.224 | 55.106 | 1.00 | 10.60 | C |
| ATOM | 2610 | O | LEU | A | 176 | 19.009 | 11.996 | 55.285 | 1.00 | 11.03 | O |
| ATOM | 2612 | N | ALA | A | 177 | 18.874 | 13.828 | 53.956 | 1.00 | 9.29 | N |
| ATOM | 2613 | CA | ALA | A | 177 | 18.364 | 13.067 | 52.816 | 1.00 | 9.08 | C |
| ATOM | 2615 | CB | ALA | A | 177 | 16.871 | 13.142 | 52.676 | 1.00 | 10.99 | C |
| ATOM | 2619 | C | ALA | A | 177 | 19.051 | 13.561 | 51.558 | 1.00 | 10.04 | C |
| ATOM | 2620 | O | ALA | A | 177 | 19.398 | 14.737 | 51.438 | 1.00 | 9.75 | O |
| ATOM | 2622 | N | PHE | A | 178 | 19.222 | 12.627 | 50.630 | 1.00 | 9.02 | N |
| ATOM | 2623 | CA | PHE | A | 178 | 19.846 | 12.880 | 49.346 | 1.00 | 9.63 | C |
| ATOM | 2625 | CB | PHE | A | 178 | 21.096 | 12.010 | 49.256 | 1.00 | 9.71 | C |
| ATOM | 2628 | CG | PHE | A | 178 | 22.014 | 12.180 | 50.397 | 1.00 | 11.34 | C |
| ATOM | 2629 | CD1 | PHE | A | 178 | 22.904 | 13.231 | 50.406 | 1.00 | 17.04 | C |
| ATOM | 2631 | CE1 | PHE | A | 178 | 23.766 | 13.393 | 51.473 | 1.00 | 19.52 | C |
| ATOM | 2633 | CZ | PHE | A | 178 | 23.687 | 12.533 | 52.567 | 1.00 | 15.86 | C |
| ATOM | 2635 | CE2 | PHE | A | 178 | 22.828 | 11.493 | 52.563 | 1.00 | 12.43 | C |
| ATOM | 2637 | CD2 | PHE | A | 178 | 21.968 | 11.319 | 51.505 | 1.00 | 11.87 | C |
| ATOM | 2639 | C | PHE | A | 178 | 18.884 | 12.509 | 48.243 | 1.00 | 10.24 | C |
| ATOM | 2640 | O | PHE | A | 178 | 18.310 | 11.419 | 48.264 | 1.00 | 10.41 | O |
| ATOM | 2642 | N | GLN | A | 179 | 18.706 | 13.407 | 47.281 | 1.00 | 9.37 | N |
| ATOM | 2643 | CA | GLN | A | 179 | 17.882 | 13.172 | 46.105 | 1.00 | 9.39 | C |
| ATOM | 2645 | CB | GLN | A | 179 | 16.841 | 14.275 | 45.937 | 1.00 | 9.56 | C |
| ATOM | 2648 | CG | GLN | A | 179 | 15.926 | 14.032 | 44.737 | 1.00 | 10.70 | C |
| ATOM | 2651 | CD | GLN | A | 179 | 15.123 | 15.238 | 44.384 | 1.00 | 14.67 | C |
| ATOM | 2652 | OE1 | GLN | A | 179 | 15.614 | 16.371 | 44.426 | 1.00 | 19.87 | O |
| ATOM | 2653 | NE2 | GLN | A | 179 | 13.835 | 15.015 | 44.063 | 1.00 | 19.39 | N |
| ATOM | 2656 | C | GLN | A | 179 | 18.780 | 13.131 | 44.862 | 1.00 | 9.70 | C |
| ATOM | 2657 | O | GLN | A | 179 | 19.507 | 14.063 | 44.582 | 1.00 | 9.12 | O |
| ATOM | 2659 | N | ASP | A | 180 | 18.681 | 12.017 | 44.151 | 1.00 | 9.12 | N |
| ATOM | 2660 | CA | ASP | A | 180 | 19.246 | 11.827 | 42.834 | 1.00 | 8.67 | C |
| ATOM | 2662 | CB | ASP | A | 180 | 19.988 | 10.495 | 42.808 | 1.00 | 9.62 | C |
| ATOM | 2665 | CG | ASP | A | 180 | 20.230 | 10.004 | 41.384 | 1.00 | 11.84 | C |
| ATOM | 2666 | OD1 | ASP | A | 180 | 21.098 | 10.598 | 40.700 | 1.00 | 9.57 | O |
| ATOM | 2667 | OD2 | ASP | A | 180 | 19.506 | 9.069 | 40.929 | 1.00 | 10.67 | O |
| ATOM | 2668 | C | ASP | A | 180 | 18.165 | 11.825 | 41.811 | 1.00 | 9.18 | C |
| ATOM | 2669 | O | ASP | A | 180 | 17.071 | 11.293 | 42.046 | 1.00 | 9.26 | O |
| ATOM | 2671 | N | GLN | A | 181 | 18.429 | 12.401 | 40.653 | 1.00 | 7.59 | N |
| ATOM | 2672 | CA | GLN | A | 181 | 17.486 | 12.287 | 39.502 | 1.00 | 9.62 | C |
| ATOM | 2674 | CB | GLN | A | 181 | 16.446 | 13.404 | 39.553 | 1.00 | 9.54 | C |
| ATOM | 2677 | CG | GLN | A | 181 | 15.294 | 13.192 | 38.629 | 1.00 | 12.83 | C |
| ATOM | 2680 | CD | GLN | A | 181 | 14.240 | 14.249 | 38.781 | 1.00 | 17.44 | C |
| ATOM | 2681 | OE1 | GLN | A | 181 | 13.142 | 13.998 | 39.296 | 1.00 | 25.22 | O |
| ATOM | 2682 | NE2 | GLN | A | 181 | 14.536 | 15.437 | 38.295 | 1.00 | 24.11 | N |
| ATOM | 2685 | C | GLN | A | 181 | 18.323 | 12.339 | 38.220 | 1.00 | 9.56 | C |
| ATOM | 2686 | O | GLN | A | 181 | 18.688 | 13.405 | 37.760 | 1.00 | 10.29 | O |
| ATOM | 2688 | N | GLY | A | 182 | 18.706 | 11.170 | 37.715 | 1.00 | 9.26 | N |
| ATOM | 2689 | CA | GLY | A | 182 | 19.458 | 11.066 | 36.463 | 1.00 | 9.98 | C |
| ATOM | 2692 | C | GLY | A | 182 | 20.962 | 11.281 | 36.539 | 1.00 | 9.35 | C |
| ATOM | 2693 | O | GLY | A | 182 | 21.615 | 11.486 | 35.523 | 1.00 | 9.38 | O |
| ATOM | 2695 | N | ALA | A | 183 | 21.531 | 11.165 | 37.740 | 1.00 | 8.87 | N |
| ATOM | 2696 | CA | ALA | A | 183 | 22.990 | 11.245 | 37.923 | 1.00 | 8.24 | C |
| ATOM | 2698 | CB | ALA | A | 183 | 23.362 | 12.143 | 39.062 | 1.00 | 8.69 | C |
| ATOM | 2702 | C | ALA | A | 183 | 23.548 | 9.855 | 38.159 | 1.00 | 8.99 | C |
| ATOM | 2703 | O | ALA | A | 183 | 22.798 | 8.874 | 38.249 | 1.00 | 10.60 | O |
| ATOM | 2705 | N | CYS | A | 184 | 24.874 | 9.772 | 38.195 | 1.00 | 7.92 | N |
| ATOM | 2706 | CA | CYS | A | 184 | 25.549 | 8.477 | 38.316 | 1.00 | 7.85 | C |
| ATOM | 2708 | CB | CYS | A | 184 | 26.054 | 8.023 | 36.956 | 1.00 | 8.58 | C |
| ATOM | 2711 | SG | CYS | A | 184 | 26.830 | 6.361 | 36.962 | 1.00 | 9.83 | S |
| ATOM | 2713 | C | CYS | A | 184 | 26.704 | 8.693 | 39.292 | 1.00 | 7.08 | C |
| ATOM | 2714 | O | CYS | A | 184 | 27.735 | 9.254 | 38.916 | 1.00 | 8.95 | O |
| ATOM | 2716 | N | MET | A | 185 | 26.516 | 8.289 | 40.536 | 1.00 | 7.67 | N |
| ATOM | 2717 | CA | MET | A | 185 | 27.400 | 8.698 | 41.620 | 1.00 | 7.79 | C |
| ATOM | 2719 | CB | MET | A | 185 | 26.891 | 9.986 | 42.242 | 1.00 | 8.31 | C |
| ATOM | 2722 | CG | MET | A | 185 | 25.509 | 9.869 | 42.942 | 1.00 | 8.09 | C |
| ATOM | 2725 | SD | MET | A | 185 | 25.067 | 11.370 | 43.893 | 1.00 | 10.10 | S |
| ATOM | 2726 | CE | MET | A | 185 | 26.262 | 11.292 | 45.178 | 1.00 | 11.25 | C |
| ATOM | 2730 | C | MET | A | 185 | 27.555 | 7.657 | 42.707 | 1.00 | 8.04 | C |
| ATOM | 2731 | O | MET | A | 185 | 26.716 | 6.768 | 42.891 | 1.00 | 7.27 | O |
| ATOM | 2733 | N | ALA | A | 186 | 28.681 | 7.761 | 43.406 | 1.00 | 7.98 | N |
| ATOM | 2734 | CA | ALA | A | 186 | 28.926 | 6.985 | 44.617 | 1.00 | 9.39 | C |
| ATOM | 2736 | CB | ALA | A | 186 | 30.175 | 6.146 | 44.499 | 1.00 | 8.65 | C |
| ATOM | 2740 | C | ALA | A | 186 | 29.118 | 7.945 | 45.786 | 1.00 | 9.09 | C |
| ATOM | 2741 | O | ALA | A | 186 | 30.032 | 8.789 | 45.742 | 1.00 | 8.21 | O |
| ATOM | 2743 | N | LEU | A | 187 | 28.282 | 7.814 | 46.817 | 1.00 | 8.78 | N |
| ATOM | 2744 | CA | LEU | A | 187 | 28.487 | 8.519 | 48.112 | 1.00 | 9.03 | C |
| ATOM | 2746 | CB | LEU | A | 187 | 27.152 | 8.751 | 48.826 | 1.00 | 9.73 | C |
| ATOM | 2749 | CG | LEU | A | 187 | 27.259 | 9.515 | 50.135 | 1.00 | 11.78 | C |
| ATOM | 2751 | CD1 | LEU | A | 187 | 27.747 | 10.946 | 49.895 | 1.00 | 13.80 | C |
| ATOM | 2755 | CD2 | LEU | A | 187 | 25.896 | 9.537 | 50.804 | 1.00 | 15.87 | C |
| ATOM | 2759 | C | LEU | A | 187 | 29.421 | 7.629 | 48.946 | 1.00 | 8.46 | C |
| ATOM | 2760 | O | LEU | A | 187 | 29.051 | 6.559 | 49.435 | 1.00 | 8.87 | O |
| ATOM | 2762 | N | LEU | A | 188 | 30.666 | 8.065 | 49.036 | 1.00 | 8.40 | N |
| ATOM | 2763 | CA | LEU | A | 188 | 31.722 | 7.307 | 49.652 | 1.00 | 8.29 | C |
| ATOM | 2765 | CB | LEU | A | 188 | 33.071 | 7.652 | 49.003 | 1.00 | 9.53 | C |
| ATOM | 2768 | CG | LEU | A | 188 | 33.166 | 7.411 | 47.482 | 1.00 | 9.10 | C |
| ATOM | 2770 | CD1 | LEU | A | 188 | 34.477 | 7.931 | 46.911 | 1.00 | 11.01 | C |
| ATOM | 2774 | CD2 | LEU | A | 188 | 33.024 | 5.950 | 47.150 | 1.00 | 11.04 | C |
| ATOM | 2778 | C | LEU | A | 188 | 31.797 | 7.477 | 51.146 | 1.00 | 8.56 | C |
| ATOM | 2779 | O | LEU | A | 188 | 32.177 | 6.551 | 51.836 | 1.00 | 9.83 | O |
| ATOM | 2781 | N | SER | A | 189 | 31.428 | 8.647 | 51.620 | 1.00 | 8.94 | N |
| ATOM | 2782 | CA | SER | A | 189 | 31.360 | 8.899 | 53.068 | 1.00 | 8.72 | C |
| ATOM | 2784 | CB | SER | A | 189 | 32.757 | 8.981 | 53.658 | 1.00 | 10.49 | C |
| ATOM | 2787 | OG | SER | A | 189 | 33.482 | 10.025 | 53.106 | 1.00 | 12.31 | O |
| ATOM | 2789 | C | SER | A | 189 | 30.585 | 10.154 | 53.420 | 1.00 | 8.61 | C |
| ATOM | 2790 | O | SER | A | 189 | 30.426 | 11.093 | 52.632 | 1.00 | 8.28 | O |
| ATOM | 2792 | N | LEU | A | 190 | 29.981 | 10.099 | 54.613 | 1.00 | 8.03 | N |
| ATOM | 2793 | CA | LEU | A | 190 | 29.319 | 11.229 | 55.254 | 1.00 | 7.40 | C |
| ATOM | 2795 | CB | LEU | A | 190 | 27.778 | 11.045 | 55.319 | 1.00 | 7.78 | C |
| ATOM | 2798 | CG | LEU | A | 190 | 27.038 | 12.050 | 56.182 | 1.00 | 8.27 | C |
| ATOM | 2800 | CD1 | LEU | A | 190 | 27.148 | 13.503 | 55.675 | 1.00 | 10.17 | C |
| ATOM | 2804 | CD2 | LEU | A | 190 | 25.540 | 11.644 | 56.344 | 1.00 | 8.94 | C |
| ATOM | 2808 | C | LEU | A | 190 | 29.895 | 11.315 | 56.674 | 1.00 | 8.35 | C |
| ATOM | 2809 | O | LEU | A | 190 | 29.892 | 10.323 | 57.437 | 1.00 | 8.74 | O |
| ATOM | 2811 | N | HIS | A | 191 | 30.306 | 12.512 | 57.072 | 1.00 | 7.60 | N |
| ATOM | 2812 | CA | HIS | A | 191 | 30.745 | 12.781 | 58.457 | 1.00 | 7.90 | C |
| ATOM | 2814 | CB | HIS | A | 191 | 32.260 | 12.962 | 58.452 | 1.00 | 8.03 | C |
| ATOM | 2817 | CG | HIS | A | 191 | 32.910 | 12.968 | 59.808 | 1.00 | 8.71 | C |
| ATOM | 2818 | ND1 | HIS | A | 191 | 34.281 | 12.895 | 59.947 | 1.00 | 11.85 | N |
| ATOM | 2820 | CE1 | HIS | A | 191 | 34.588 | 12.927 | 61.239 | 1.00 | 11.49 | C |
| ATOM | 2822 | NE2 | HIS | A | 191 | 33.467 | 13.013 | 61.940 | 1.00 | 8.44 | N |
| ATOM | 2824 | CD2 | HIS | A | 191 | 32.403 | 13.030 | 61.069 | 1.00 | 8.15 | C |
| ATOM | 2826 | C | HIS | A | 191 | 30.071 | 14.048 | 58.935 | 1.00 | 8.00 | C |
| ATOM | 2827 | O | HIS | A | 191 | 30.204 | 15.094 | 58.323 | 1.00 | 9.66 | O |
| ATOM | 2829 | N | LEU | A | 192 | 29.224 | 13.867 | 59.943 | 1.00 | 8.43 | N |
| ATOM | 2830 | CA | LEU | A | 192 | 28.549 | 14.963 | 60.619 | 1.00 | 9.08 | C |
| ATOM | 2832 | CB | LEU | A | 192 | 27.099 | 14.577 | 60.975 | 1.00 | 10.10 | C |
| ATOM | 2835 | CG | LEU | A | 192 | 26.167 | 14.276 | 59.809 | 1.00 | 11.69 | C |
| ATOM | 2837 | CD1 | LEU | A | 192 | 24.869 | 13.680 | 60.288 | 1.00 | 12.31 | C |
| ATOM | 2841 | CD2 | LEU | A | 192 | 25.899 | 15.492 | 59.016 | 1.00 | 13.67 | C |
| ATOM | 2845 | C | LEU | A | 192 | 29.293 | 15.297 | 61.893 | 1.00 | 7.94 | C |
| ATOM | 2846 | O | LEU | A | 192 | 29.645 | 14.397 | 62.676 | 1.00 | 8.07 | O |
| ATOM | 2848 | N | PHE | A | 193 | 29.556 | 16.583 | 62.131 | 1.00 | 8.07 | N |
| ATOM | 2849 | CA | PHE | A | 193 | 30.262 | 17.006 | 63.330 | 1.00 | 7.59 | C |
| ATOM | 2851 | CB | PHE | A | 193 | 31.797 | 16.907 | 63.174 | 1.00 | 7.55 | C |
| ATOM | 2854 | CG | PHE | A | 193 | 32.371 | 17.852 | 62.160 | 1.00 | 9.51 | C |
| ATOM | 2855 | CD1 | PHE | A | 193 | 32.741 | 19.151 | 62.540 | 1.00 | 9.02 | C |
| ATOM | 2857 | CE1 | PHE | A | 193 | 32.253 | 20.032 | 61.585 | 1.00 | 9.94 | C |
| ATOM | 2859 | CZ | PHE | A | 193 | 33.398 | 19.610 | 60.285 | 1.00 | 8.63 | C |
| ATOM | 2861 | CE2 | PHE | A | 193 | 33.023 | 18.360 | 59.896 | 1.00 | 11.72 | C |
| ATOM | 2863 | CD2 | PHE | A | 193 | 32.508 | 17.471 | 60.815 | 1.00 | 10.23 | C |
| ATOM | 2865 | C | PHE | A | 193 | 29.835 | 18.415 | 63.705 | 1.00 | 8.62 | C |
| ATOM | 2866 | O | PHE | A | 193 | 29.228 | 19.118 | 62.894 | 1.00 | 8.78 | O |
| ATOM | 2868 | N | TYR | A | 194 | 30.141 | 18.800 | 64.938 | 1.00 | 8.07 | N |
| ATOM | 2869 | CA | TYR | A | 194 | 29.910 | 20.193 | 65.386 | 1.00 | 8.73 | C |
| ATOM | 2871 | CB | TYR | A | 194 | 28.589 | 20.329 | 66.175 | 1.00 | 9.62 | C |
| ATOM | 2874 | CG | TYR | A | 194 | 28.510 | 19.615 | 67.517 | 1.00 | 8.79 | C |
| ATOM | 2875 | CD1 | TYR | A | 194 | 28.578 | 20.311 | 68.731 | 1.00 | 11.90 | C |
| ATOM | 2877 | CE1 | TYR | A | 194 | 28.489 | 19.623 | 69.948 | 1.00 | 12.30 | C |
| ATOM | 2879 | CZ | TYR | A | 194 | 28.319 | 18.252 | 69.969 | 1.00 | 13.69 | C |
| ATOM | 2880 | OH | TYR | A | 194 | 28.213 | 17.570 | 71.188 | 1.00 | 16.83 | O |
| ATOM | 2882 | CE2 | TYR | A | 194 | 28.269 | 17.553 | 68.770 | 1.00 | 13.74 | C |
| ATOM | 2884 | CD2 | TYR | A | 194 | 28.363 | 18.232 | 67.581 | 1.00 | 10.43 | C |
| ATOM | 2886 | C | TYR | A | 194 | 31.068 | 20.698 | 66.219 | 1.00 | 9.43 | C |
| ATOM | 2887 | O | TYR | A | 194 | 31.923 | 19.929 | 66.662 | 1.00 | 9.21 | O |
| ATOM | 2889 | N | LYS | A | 195 | 31.104 | 22.018 | 66.437 | 1.00 | 10.08 | N |
| ATOM | 2890 | CA | LYS | A | 195 | 32.157 | 22.657 | 67.262 | 1.00 | 11.96 | C |
| ATOM | 2892 | CB | LYS | A | 195 | 32.548 | 24.044 | 66.674 | 1.00 | 13.33 | C |
| ATOM | 2899 | C | LYS | A | 195 | 31.648 | 22.754 | 68.692 | 1.00 | 12.87 | C |
| ATOM | 2900 | O | LYS | A | 195 | 30.573 | 23.352 | 68.946 | 1.00 | 12.94 | O |
| ATOM | 2902 | N | LYS | A | 196 | 32.383 | 22.113 | 69.611 | 1.00 | 14.64 | N |
| ATOM | 2903 | CA | LYS | A | 196 | 32.037 | 22.049 | 71.058 | 1.00 | 16.38 | C |
| ATOM | 2905 | CB | LYS | A | 196 | 32.025 | 20.593 | 71.561 | 1.00 | 16.65 | C |
| ATOM | 2908 | CG | LYS | A | 196 | 31.247 | 20.318 | 72.849 | 1.00 | 19.41 | C |
| ATOM | 2911 | CD | LYS | A | 196 | 31.368 | 18.853 | 73.224 | 1.00 | 20.76 | C |
| ATOM | 2914 | CE | LYS | A | 196 | 30.555 | 18.496 | 74.506 | 1.00 | 21.67 | C |
| ATOM | 2917 | NZ | LYS | A | 196 | 31.158 | 18.990 | 75.740 | 1.00 | 26.89 | N |
| ATOM | 2921 | C | LYS | A | 196 | 33.054 | 22.847 | 71.831 | 1.00 | 17.46 | C |
| ATOM | 2922 | O | LYS | A | 196 | 34.262 | 22.740 | 71.576 | 1.00 | 17.70 | O |
| ATOM | 2924 | N | ASN | A | 251 | 43.160 | 4.312 | 44.067 | 1.00 | 22.03 | N |
| ATOM | 2925 | CA | ASN | A | 251 | 42.141 | 5.288 | 44.481 | 1.00 | 21.06 | C |
| ATOM | 2927 | CB | ASN | A | 251 | 42.373 | 6.675 | 43.819 | 1.00 | 21.54 | C |
| ATOM | 2930 | CG | ASN | P | 251 | 41.459 | 7.820 | 44.415 | 1.00 | 23.63 | C |
| ATOM | 2931 | OD1 | ASN | P | 251 | 41.322 | 8.907 | 43.808 | 1.00 | 25.42 | O |
| ATOM | 2932 | ND2 | ASN | P | 251 | 40.886 | 7.585 | 45.615 | 1.00 | 22.93 | N |
| ATOM | 2935 | C | ASN | P | 251 | 40.751 | 4.645 | 44.149 | 1.00 | 19.88 | C |
| ATOM | 2936 | O | ASN | P | 251 | 40.317 | 4.563 | 42.982 | 1.00 | 21.13 | O |
| ATOM | 2940 | N | TYR | P | 252 | 40.098 | 4.148 | 45.183 | 1.00 | 15.93 | N |
| ATOM | 2941 | CA | TYR | P | 252 | 38.770 | 3.589 | 45.052 | 1.00 | 14.57 | C |
| ATOM | 2943 | CB | TYR | P | 252 | 38.328 | 2.996 | 46.409 | 1.00 | 14.90 | C |
| ATOM | 2946 | CG | TYR | P | 252 | 36.982 | 2.348 | 46.341 | 1.00 | 13.17 | C |
| ATOM | 2947 | CD1 | TYR | P | 252 | 36.840 | 0.998 | 45.980 | 1.00 | 15.56 | C |
| ATOM | 2949 | CE1 | TYR | P | 252 | 35.610 | 0.399 | 45.879 | 1.00 | 14.96 | C |
| ATOM | 2951 | CZ | TYR | P | 252 | 34.472 | 1.148 | 46.129 | 1.00 | 15.22 | C |
| ATOM | 2952 | OH | TYR | P | 252 | 33.208 | 0.610 | 46.073 | 1.00 | 13.93 | O |
| ATOM | 2954 | CE2 | TYR | P | 252 | 34.589 | 2.455 | 46.537 | 1.00 | 14.09 | C |
| ATOM | 2956 | CD2 | TYR | P | 252 | 35.852 | 3.053 | 46.616 | 1.00 | 13.95 | C |
| ATOM | 2958 | C | TYR | P | 252 | 37.771 | 4.690 | 44.624 | 1.00 | 13.53 | C |
| ATOM | 2959 | O | TYR | P | 252 | 37.636 | 5.714 | 45.317 | 1.00 | 14.00 | O |
| ATOM | 2961 | N | LEU | P | 253 | 37.072 | 4.461 | 43.522 | 1.00 | 12.91 | N |
| ATOM | 2962 | CA | LEU | P | 253 | 36.060 | 5.416 | 43.022 | 1.00 | 11.40 | C |
| ATOM | 2964 | CB | LEU | P | 253 | 36.359 | 5.843 | 41.580 | 1.00 | 11.85 | C |
| ATOM | 2967 | CG | LEU | P | 253 | 37.702 | 6.574 | 41.405 | 1.00 | 11.52 | C |
| ATOM | 2969 | CD1 | LEU | P | 253 | 38.051 | 6.861 | 39.938 | 1.00 | 14.14 | C |
| ATOM | 2973 | CD2 | LEU | P | 253 | 37.735 | 7.838 | 42.176 | 1.00 | 13.77 | C |
| ATOM | 2977 | C | LEU | P | 253 | 34.642 | 4.844 | 43.115 | 1.00 | 12.69 | C |
| ATOM | 2978 | O | LEU | P | 253 | 33.716 | 5.576 | 43.386 | 1.00 | 13.84 | O |
| ATOM | 2980 | N | PHE | P | 254 | 34.471 | 3.549 | 42.831 | 1.00 | 11.86 | N |
| ATOM | 2981 | CA | PHE | P | 254 | 33.198 | 2.865 | 42.874 | 1.00 | 12.02 | C |
| ATOM | 2983 | CB | PHE | P | 254 | 32.261 | 3.307 | 41.741 | 1.00 | 13.21 | C |
| ATOM | 2986 | CG | PHE | P | 254 | 32.806 | 3.116 | 40.346 | 1.00 | 13.27 | C |
| ATOM | 2987 | CD1 | PHE | P | 254 | 32.742 | 1.887 | 39.712 | 1.00 | 15.51 | C |
| ATOM | 2989 | CE1 | PHE | P | 254 | 33.218 | 1.718 | 38.441 | 1.00 | 17.28 | C |
| ATOM | 2991 | CZ | PHE | P | 254 | 33.784 | 2.777 | 37.760 | 1.00 | 18.13 | C |
| ATOM | 2993 | CE2 | PHE | P | 254 | 33.848 | 4.025 | 38.396 | 1.00 | 17.44 | C |
| ATOM | 2995 | CD2 | PHE | P | 254 | 33.380 | 4.176 | 39.674 | 1.00 | 15.88 | C |
| ATOM | 2997 | C | PHE | P | 254 | 33.416 | 1.353 | 42.848 | 1.00 | 11.55 | C |
| ATOM | 2998 | O | PHE | P | 254 | 34.547 | 0.864 | 42.674 | 1.00 | 12.47 | O |
| ATOM | 3000 | N | SER | P | 255 | 32.317 | 0.633 | 43.050 | 1.00 | 13.41 | N |
| ATOM | 3001 | CA | SER | P | 255 | 32.350 | −0.813 | 43.277 | 1.00 | 13.05 | C |
| ATOM | 3003 | CB | SER | P | 255 | 31.111 | −1.266 | 44.041 | 1.00 | 13.36 | C |
| ATOM | 3006 | OG | SER | P | 255 | 30.995 | −2.681 | 44.116 | 1.00 | 14.10 | O |
| ATOM | 3008 | C | SER | P | 255 | 32.362 | −1.565 | 41.971 | 1.00 | 14.28 | C |
| ATOM | 3009 | O | SER | P | 255 | 31.717 | −1.153 | 41.010 | 1.00 | 15.30 | O |
| ATOM | 3011 | N | PRO | P | 256 | 33.069 | −2.701 | 41.933 | 1.00 | 14.39 | N |
| ATOM | 3012 | CA | PRO | P | 256 | 32.933 | −3.542 | 40.748 | 1.00 | 14.47 | C |
| ATOM | 3014 | CB | PRO | P | 256 | 34.011 | −4.593 | 40.928 | 1.00 | 15.06 | C |
| ATOM | 3017 | CG | PRO | P | 256 | 34.313 | −4.609 | 42.373 | 1.00 | 15.97 | C |
| ATOM | 3020 | CD | PRO | P | 256 | 33.979 | −3.265 | 42.941 | 1.00 | 15.21 | C |
| ATOM | 3023 | C | PRO | P | 256 | 31.574 | −4.220 | 40.607 | 1.00 | 14.15 | C |
| ATOM | 3024 | O | PRO | P | 256 | 31.268 | −4.758 | 39.529 | 1.00 | 14.96 | O |
| ATOM | 3025 | N | ASN | P | 257 | 30.767 | −4.179 | 41.655 | 1.00 | 13.76 | N |
| ATOM | 3026 | CA | ASN | P | 257 | 29.495 | −4.927 | 41.742 | 1.00 | 14.91 | C |
| ATOM | 3028 | CB | ASN | P | 257 | 29.528 | −5.761 | 43.019 | 1.00 | 16.24 | C |
| ATOM | 3031 | CG | ASN | P | 257 | 30.710 | −6.703 | 43.075 | 1.00 | 19.43 | C |
| ATOM | 3032 | OD1 | ASN | P | 257 | 31.147 | −7.233 | 42.056 | 1.00 | 19.88 | O |
| ATOM | 3033 | ND2 | ASN | P | 257 | 31.217 | −6.940 | 44.283 | 1.00 | 22.73 | N |
| ATOM | 3036 | C | ASN | P | 257 | 28.233 | −4.088 | 41.832 | 1.00 | 14.65 | C |
| ATOM | 3037 | O | ASN | P | 257 | 27.166 | −4.633 | 42.116 | 1.00 | 14.56 | O |
| ATOM | 3039 | N | GLY | P | 258 | 28.349 | −2.782 | 41.612 | 1.00 | 13.71 | N |
| ATOM | 3040 | CA | GLY | P | 258 | 27.226 | −1.869 | 41.782 | 1.00 | 13.39 | C |
| ATOM | 3043 | C | GLY | P | 258 | 26.633 | −1.319 | 40.490 | 1.00 | 13.17 | C |
| ATOM | 3044 | O | GLY | P | 258 | 26.963 | −1.794 | 39.408 | 1.00 | 13.88 | O |
| ATOM | 3046 | N | PRO | P | 259 | 25.669 | −0.356 | 40.634 | 1.00 | 13.28 | N |
| ATOM | 3047 | CA | PRO | P | 259 | 24.924 | 0.159 | 39.482 | 1.00 | 13.62 | C |
| ATOM | 3049 | CB | PRO | P | 259 | 23.826 | 1.046 | 40.106 | 1.00 | 14.38 | C |
| ATOM | 3052 | CG | PRO | P | 259 | 24.209 | 1.284 | 41.465 | 1.00 | 14.55 | C |
| ATOM | 3055 | CD | PRO | P | 259 | 25.169 | 0.197 | 41.902 | 1.00 | 14.06 | C |
| ATOM | 3058 | C | PRO | P | 259 | 25.791 | 0.979 | 38.544 | 1.00 | 13.22 | C |
| ATOM | 3059 | O | PRO | P | 259 | 25.481 | 1.101 | 37.327 | 1.00 | 13.10 | O |
| ATOM | 3060 | N | ILE | P | 260 | 26.879 | 1.524 | 39.081 | 1.00 | 13.54 | N |
| ATOM | 3061 | CA | ILE | P | 260 | 27.816 | 2.249 | 38.235 | 1.00 | 13.31 | C |
| ATOM | 3063 | CB | ILE | P | 260 | 28.826 | 3.067 | 39.067 | 1.00 | 12.69 | C |
| ATOM | 3065 | CG1 | ILE | P | 260 | 28.069 | 4.128 | 39.881 | 1.00 | 12.98 | C |
| ATOM | 3068 | CD1 | ILE | P | 260 | 28.883 | 4.824 | 40.935 | 1.00 | 14.32 | C |
| ATOM | 3072 | CG2 | ILE | P | 260 | 29.810 | 3.759 | 38.138 | 1.00 | 13.16 | C |
| ATOM | 3076 | C | ILE | P | 260 | 28.538 | 1.293 | 37.279 | 1.00 | 13.17 | C |
| ATOM | 3077 | O | ILE | P | 260 | 28.536 | 1.480 | 36.056 | 1.00 | 12.37 | O |
| ATOM | 3079 | N | ALA | P | 261 | 29.132 | 0.236 | 37.826 | 1.00 | 13.12 | N |
| ATOM | 3080 | CA | ALA | P | 261 | 29.780 | −0.765 | 36.998 | 1.00 | 13.37 | C |
| ATOM | 3082 | CB | ALA | P | 261 | 30.371 | −1.875 | 37.861 | 1.00 | 14.02 | C |
| ATOM | 3086 | C | ALA | P | 261 | 28.830 | −1.344 | 35.955 | 1.00 | 14.14 | C |
| ATOM | 3087 | O | ALA | P | 261 | 29.239 | −1.651 | 34.831 | 1.00 | 15.28 | O |
| ATOM | 3089 | N | ARG | P | 262 | 27.566 | −1.517 | 36.326 | 1.00 | 13.51 | N |
| ATOM | 3090 | CA | ARG | P | 262 | 26.558 | −2.069 | 35.421 | 1.00 | 14.66 | C |
| ATOM | 3092 | CB | ARG | P | 262 | 25.272 | −2.428 | 36.205 | 1.00 | 15.22 | C |
| ATOM | 3095 | CG | ARG | P | 262 | 25.455 | −3.668 | 37.078 | 1.00 | 16.13 | C |
| ATOM | 3098 | CD | ARG | P | 262 | 24.124 | −4.259 | 37.537 | 1.00 | 16.60 | C |
| ATOM | 3101 | NE | ARG | P | 262 | 23.470 | −3.395 | 38.513 | 1.00 | 17.89 | N |
| ATOM | 3103 | CZ | ARG | P | 262 | 23.689 | −3.419 | 39.816 | 1.00 | 18.17 | C |
| ATOM | 3104 | NH1 | ARG | P | 262 | 24.573 | −4.225 | 40.375 | 1.00 | 17.86 | N |
| ATOM | 3107 | NH2 | ARG | P | 262 | 23.025 | −2.582 | 40.577 | 1.00 | 20.47 | N |
| ATOM | 3110 | C | ARG | P | 262 | 26.221 | −1.156 | 34.242 | 1.00 | 15.24 | C |
| ATOM | 3111 | O | ARG | P | 262 | 25.629 | −1.634 | 33.239 | 1.00 | 15.81 | O |
| ATOM | 3113 | N | ALA | P | 263 | 26.605 | 0.121 | 34.320 | 1.00 | 16.39 | N |
| ATOM | 3114 | CA | ALA | P | 263 | 26.379 | 1.030 | 33.211 | 1.00 | 17.38 | C |
| ATOM | 3116 | CB | ALA | P | 263 | 26.783 | 2.445 | 33.547 | 1.00 | 17.32 | C |
| ATOM | 3120 | C | ALA | P | 263 | 27.118 | 0.550 | 31.947 | 1.00 | 18.17 | C |
| ATOM | 3121 | O | ALA | P | 263 | 26.744 | 0.929 | 30.831 | 1.00 | 18.92 | O |
| ATOM | 3123 | N | TRP | P | 264 | 28.202 | −0.202 | 32.111 | 1.00 | 18.44 | N |
| ATOM | 3124 | CA | TRP | P | 264 | 29.019 | −0.654 | 30.952 | 1.00 | 19.37 | C |
| ATOM | 3126 | CB | TRP | P | 264 | 30.510 | −0.370 | 31.200 | 1.00 | 20.30 | C |
| ATOM | 3129 | CG | TRP | P | 264 | 30.730 | 1.090 | 31.441 | 1.00 | 20.52 | C |
| ATOM | 3130 | CD1 | TRP | P | 264 | 30.626 | 2.093 | 30.530 | 1.00 | 24.02 | C |
| ATOM | 3132 | NE1 | TRP | P | 264 | 30.859 | 3.305 | 31.129 | 1.00 | 21.70 | N |
| ATOM | 3134 | CE2 | TRP | P | 264 | 31.143 | 3.103 | 32.440 | 1.00 | 23.96 | C |
| ATOM | 3135 | CD2 | TRP | P | 264 | 31.054 | 1.711 | 32.678 | 1.00 | 20.21 | C |
| ATOM | 3136 | CE3 | TRP | P | 264 | 31.337 | 1.231 | 33.950 | 1.00 | 21.14 | C |
| ATOM | 3138 | CZ3 | TRP | P | 264 | 31.642 | 2.143 | 34.959 | 1.00 | 23.62 | C |
| ATOM | 3140 | CH2 | TRP | P | 264 | 31.684 | 3.522 | 34.702 | 1.00 | 23.52 | C |
| ATOM | 3142 | CZ2 | TRP | P | 264 | 31.423 | 4.019 | 33.450 | 1.00 | 20.98 | C |
| ATOM | 3144 | C | TRP | P | 264 | 28.773 | −2.117 | 30.645 | 1.00 | 20.85 | C |
| ATOM | 3145 | O | TRP | P | 264 | 27.899 | −2.714 | 31.272 | 1.00 | 22.43 | O |
| ATOM | 3147 | OXT | TRP | P | 264 | 29.405 | −2.712 | 29.746 | 1.00 | 21.74 | O |
| ATOM | 3148 | O | HOH | X | 301 | 34.430 | 11.979 | 40.417 | 1.00 | 7.01 | O |
| ATOM | 3151 | O | HOH | X | 302 | 8.396 | 11.854 | 46.295 | 1.00 | 10.84 | O |
| ATOM | 3154 | O | HOH | X | 304 | 31.629 | 5.424 | 54.367 | 1.00 | 7.78 | O |
| ATOM | 3157 | O | HOH | X | 305 | 29.393 | 0.001 | 40.716 | 1.00 | 11.67 | O |
| ATOM | 3160 | O | HOH | X | 306 | 26.504 | 11.508 | 36.590 | 1.00 | 11.86 | O |
| ATOM | 3163 | O | HOH | X | 308 | 31.743 | 1.752 | 56.936 | 1.00 | 11.18 | O |
| ATOM | 3166 | O | HOH | X | 309 | 5.324 | 13.803 | 56.433 | 1.00 | 10.31 | O |
| ATOM | 3169 | O | HOH | X | 310 | 10.666 | 3.747 | 51.782 | 1.00 | 11.18 | O |
| ATOM | 3172 | O | HOH | X | 311 | 8.900 | 13.123 | 61.479 | 1.00 | 17.18 | O |
| ATOM | 3175 | O | HOH | X | 312 | 11.707 | 10.700 | 54.483 | 1.00 | 6.76 | O |
| ATOM | 3178 | O | HOH | X | 313 | 15.905 | 6.703 | 58.672 | 1.00 | 13.28 | O |
| ATOM | 3181 | O | HOH | X | 314 | 35.962 | 18.454 | 45.448 | 1.00 | 12.13 | O |
| ATOM | 3184 | O | HOH | X | 315 | 4.724 | 20.141 | 50.518 | 1.00 | 13.74 | O |
| ATOM | 3187 | O | HOH | X | 316 | 20.949 | 2.851 | 39.943 | 1.00 | 12.79 | O |
| ATOM | 3190 | O | HOH | X | 317 | 17.280 | 8.646 | 37.970 | 1.00 | 11.75 | O |
| ATOM | 3193 | O | HOH | X | 318 | 32.871 | 12.284 | 54.535 | 1.00 | 8.88 | O |
| ATOM | 3196 | O | HOH | X | 319 | 38.982 | 4.909 | 31.022 | 1.00 | 12.42 | O |
| ATOM | 3199 | O | HOH | X | 320 | 9.426 | 13.453 | 65.971 | 1.00 | 9.90 | O |
| ATOM | 3202 | O | HOH | X | 322 | 31.148 | 2.318 | 46.180 | 1.00 | 13.63 | O |
| ATOM | 3205 | O | HOH | X | 324 | 13.896 | 24.311 | 51.333 | 1.00 | 12.28 | O |
| ATOM | 3208 | O | HOH | X | 325 | 40.919 | −4.284 | 28.002 | 1.00 | 15.70 | O |
| ATOM | 3211 | O | HOH | X | 328 | 11.597 | 3.904 | 58.174 | 1.00 | 15.64 | O |
| ATOM | 3214 | O | HOH | X | 329 | 7.013 | 21.318 | 64.262 | 1.00 | 17.19 | O |
| ATOM | 3217 | O | HOH | X | 330 | 36.700 | 16.628 | 35.841 | 1.00 | 12.75 | O |
| ATOM | 3220 | O | HOH | X | 331 | 35.919 | 8.860 | 52.061 | 1.00 | 14.21 | O |
| ATOM | 3223 | O | HOH | X | 333 | 33.936 | 13.026 | 68.542 | 1.00 | 15.84 | O |
| ATOM | 3226 | O | HOH | X | 334 | 28.439 | 18.984 | 38.377 | 1.00 | 15.56 | O |
| ATOM | 3229 | O | HOH | X | 335 | 38.475 | 17.080 | 45.414 | 1.00 | 9.88 | O |
| ATOM | 3232 | O | HOH | X | 336 | 29.059 | 23.743 | 65.349 | 1.00 | 13.08 | O |
| ATOM | 3235 | O | HOH | X | 337 | 35.124 | 6.275 | 51.954 | 1.00 | 13.92 | O |
| ATOM | 3238 | O | HOH | X | 338 | 7.219 | 2.126 | 57.698 | 1.00 | 19.91 | O |
| ATOM | 3241 | O | HOH | X | 339 | 9.763 | 14.968 | 45.726 | 1.00 | 14.47 | O |
| ATOM | 3244 | O | HOH | X | 340 | 13.387 | 8.617 | 69.453 | 1.00 | 16.22 | O |
| ATOM | 3247 | O | HOH | X | 341 | 10.796 | 17.916 | 45.998 | 1.00 | 16.33 | O |
| ATOM | 3250 | O | HOH | X | 342 | 18.031 | 18.962 | 65.202 | 1.00 | 15.58 | O |
| ATOM | 3253 | O | HOH | X | 344 | 36.069 | 5.290 | 49.609 | 1.00 | 17.02 | O |
| ATOM | 3256 | O | HOH | X | 345 | 29.619 | 18.922 | 35.833 | 1.00 | 22.31 | O |
| ATOM | 3259 | O | HOH | X | 346 | 24.143 | 18.471 | 35.696 | 1.00 | 19.85 | O |
| ATOM | 3262 | O | HOH | X | 348 | 21.129 | −1.803 | 49.194 | 1.00 | 17.90 | O |
| ATOM | 3265 | O | HOH | X | 349 | 38.093 | 13.627 | 38.055 | 1.00 | 14.45 | O |
| ATOM | 3268 | O | HOH | X | 350 | 10.253 | 11.006 | 40.462 | 1.00 | 18.46 | O |
| ATOM | 3271 | O | HOH | X | 351 | 10.793 | 13.130 | 44.082 | 1.00 | 22.46 | O |
| ATOM | 3274 | O | HOH | X | 352 | 13.603 | 19.738 | 71.429 | 1.00 | 20.67 | O |
| ATOM | 3277 | O | HOH | X | 353 | 7.557 | 20.765 | 60.119 | 1.00 | 17.34 | O |
| ATOM | 3280 | O | HOH | X | 354 | 13.586 | 4.527 | 59.705 | 1.00 | 20.41 | O |
| ATOM | 3283 | O | HOH | X | 355 | 37.530 | 2.113 | 41.859 | 1.00 | 24.82 | O |
| ATOM | 3286 | O | HOH | X | 356 | 16.469 | 24.270 | 60.869 | 1.00 | 23.54 | O |
| ATOM | 3289 | O | HOH | X | 357 | 29.936 | 18.089 | 32.080 | 1.00 | 26.88 | O |
| ATOM | 3292 | O | HOH | X | 358 | 19.795 | 3.083 | 33.799 | 1.00 | 28.78 | O |
| ATOM | 3295 | O | HOH | X | 359 | 38.291 | 20.499 | 44.138 | 1.00 | 22.25 | O |
| ATOM | 3298 | O | HOH | X | 360 | 20.041 | 0.614 | 52.967 | 1.00 | 18.78 | O |
| ATOM | 3301 | O | HOH | X | 361 | 29.369 | 0.094 | 63.771 | 1.00 | 21.56 | O |
| ATOM | 3304 | O | HOH | X | 362 | 29.228 | 14.780 | 30.295 | 1.00 | 16.98 | O |
| ATOM | 3307 | O | HOH | X | 363 | 28.984 | 12.621 | 69.804 | 1.00 | 21.48 | O |
| ATOM | 3310 | O | HOH | X | 364 | 24.115 | 11.918 | 68.606 | 1.00 | 18.14 | O |
| ATOM | 3313 | O | HOH | X | 365 | 37.961 | 6.598 | 47.868 | 1.00 | 16.76 | O |
| ATOM | 3316 | O | HOH | X | 366 | 34.891 | 20.347 | 52.378 | 1.00 | 22.38 | O |
| ATOM | 3319 | O | HOH | X | 367 | 31.698 | 28.109 | 59.447 | 1.00 | 28.93 | O |
| ATOM | 3322 | O | HOH | X | 368 | 21.261 | 19.711 | 38.455 | 1.00 | 28.25 | O |
| ATOM | 3325 | O | HOH | X | 369 | 30.748 | 20.856 | 45.143 | 1.00 | 28.82 | O |
| ATOM | 3328 | O | HOH | X | 370 | 33.678 | 19.116 | 43.639 | 1.00 | 19.55 | O |
| ATOM | 3331 | O | HOH | X | 371 | 25.018 | 10.141 | 66.819 | 1.00 | 19.92 | O |
| ATOM | 3334 | O | HOH | X | 372 | 26.928 | 24.827 | 67.182 | 1.00 | 28.42 | O |
| ATOM | 3337 | O | HOH | X | 373 | 32.538 | 19.487 | 36.343 | 1.00 | 21.66 | O |
| ATOM | 3340 | O | HOH | X | 374 | 17.229 | 0.902 | 47.869 | 1.00 | 22.42 | O |
| ATOM | 3343 | O | HOH | X | 375 | 19.360 | 15.924 | 71.231 | 1.00 | 27.13 | O |
| ATOM | 3346 | O | HOH | X | 376 | 26.441 | −6.252 | 39.178 | 1.00 | 25.00 | O |
| ATOM | 3349 | O | HOH | X | 377 | 39.699 | 7.545 | 31.593 | 1.00 | 20.60 | O |
| ATOM | 3352 | O | HOH | X | 378 | 7.020 | 9.932 | 60.846 | 1.00 | 28.81 | O |
| ATOM | 3355 | O | HOH | X | 379 | 32.445 | −1.887 | 56.117 | 1.00 | 29.10 | O |
| ATOM | 3358 | O | HOH | X | 380 | 10.504 | −0.374 | 38.640 | 1.00 | 31.66 | O |
| ATOM | 3361 | O | HOH | X | 381 | 38.643 | −4.052 | 56.330 | 1.00 | 28.25 | O |
| ATOM | 3364 | O | HOH | X | 382 | 20.319 | 10.149 | 30.622 | 1.00 | 28.71 | O |
| ATOM | 3367 | O | HOH | X | 383 | 16.249 | 21.410 | 43.896 | 1.00 | 33.25 | O |
| ATOM | 3370 | O | HOH | X | 384 | 27.044 | 18.832 | 73.411 | 1.00 | 26.07 | O |
| ATOM | 3373 | O | HOH | X | 385 | 17.653 | 5.995 | 63.119 | 1.00 | 20.40 | O |
| ATOM | 3376 | O | HOH | X | 386 | 20.667 | 25.167 | 57.089 | 1.00 | 26.80 | O |
| ATOM | 3379 | O | HOH | X | 387 | 24.565 | 25.212 | 44.311 | 1.00 | 27.12 | O |
| ATOM | 3382 | O | HOH | X | 388 | 13.295 | 12.403 | 71.678 | 1.00 | 23.39 | O |
| ATOM | 3385 | O | HOH | X | 389 | 31.967 | −3.677 | 46.437 | 1.00 | 20.85 | O |
| ATOM | 3388 | O | HOH | X | 400 | 23.013 | 1.080 | 36.088 | 1.00 | 20.68 | O |
| ATOM | 3391 | O | HOH | X | 401 | 13.803 | 12.839 | 31.578 | 1.00 | 47.10 | O |
| ATOM | 3394 | O | HOH | X | 402 | 35.493 | −3.707 | 50.617 | 1.00 | 28.70 | O |
| ATOM | 3397 | O | HOH | X | 403 | 11.319 | 24.471 | 65.742 | 1.00 | 31.09 | O |
| ATOM | 3400 | O | HOH | X | 404 | 6.899 | 22.877 | 47.846 | 1.00 | 28.38 | O |
| ATOM | 3403 | O | HOH | X | 405 | 40.035 | 11.449 | 34.241 | 1.00 | 29.40 | O |
| ATOM | 3406 | O | HOH | X | 407 | 33.019 | 22.565 | 50.833 | 1.00 | 18.50 | O |
| ATOM | 3409 | O | HOH | X | 410 | 19.785 | 18.612 | 72.020 | 1.00 | 35.87 | O |
| ATOM | 3412 | O | HOH | X | 411 | 42.301 | 4.024 | 31.951 | 1.00 | 16.71 | O |
| ATOM | 3415 | O | HOH | X | 412 | 3.955 | 5.909 | 56.974 | 1.00 | 32.26 | O |
| ATOM | 3418 | O | HOH | X | 413 | 13.763 | 3.826 | 37.534 | 1.00 | 27.73 | O |
| ATOM | 3421 | O | HOH | X | 414 | 31.801 | −2.267 | 34.382 | 1.00 | 39.99 | O |
| ATOM | 3424 | O | HOH | X | 415 | 15.515 | 6.216 | 61.471 | 1.00 | 27.52 | O |
| ATOM | 3427 | O | HOH | X | 416 | 15.020 | 24.864 | 64.030 | 1.00 | 36.11 | O |
| ATOM | 3430 | O | HOH | X | 417 | 12.455 | 5.600 | 62.319 | 1.00 | 24.57 | O |
| ATOM | 3433 | O | HOH | X | 419 | 25.773 | 23.290 | 42.244 | 1.00 | 41.56 | O |
| ATOM | 3436 | O | HOH | X | 420 | 41.722 | 7.186 | 39.109 | 1.00 | 29.19 | O |
| ATOM | 3439 | O | HOH | X | 421 | 25.130 | 3.183 | 30.595 | 1.00 | 30.39 | O |
| ATOM | 3442 | O | HOH | X | 422 | 37.744 | 3.789 | 58.380 | 1.00 | 40.22 | O |
| ATOM | 3445 | O | HOH | X | 423 | 22.867 | 0.681 | 61.281 | 1.00 | 39.84 | O |
| ATOM | 3448 | O | HOH | X | 424 | 42.153 | 6.744 | 60.740 | 1.00 | 23.29 | O |
| ATOM | 3451 | O | HOH | X | 426 | 20.288 | 8.153 | 38.618 | 1.00 | 9.57 | O |
| ATOM | 3454 | O | HOH | X | 427 | 35.253 | 14.363 | 31.838 | 1.00 | 50.83 | O |
| ATOM | 3457 | O | HOH | X | 428 | 34.212 | 1.381 | 64.196 | 1.00 | 27.07 | O |
| ATOM | 3460 | O | HOH | X | 429 | 14.434 | 1.392 | 52.833 | 1.00 | 22.25 | O |
| ATOM | 3463 | O | HOH | X | 430 | 22.194 | 26.212 | 52.485 | 1.00 | 32.80 | O |
| ATOM | 3466 | O | HOH | X | 432 | 9.238 | 16.975 | 65.774 | 1.00 | 13.96 | O |
| ATOM | 3469 | O | HOH | X | 433 | 11.593 | 1.653 | 53.574 | 1.00 | 16.99 | O |
| ATOM | 3472 | O | HOH | X | 434 | 14.051 | 4.174 | 43.951 | 1.00 | 20.14 | O |
| ATOM | 3475 | O | HOH | X | 435 | 7.175 | 15.078 | 45.065 | 1.00 | 18.73 | O |
| ATOM | 3478 | O | HOH | X | 437 | 11.480 | 1.805 | 56.261 | 1.00 | 18.06 | O |
| ATOM | 3481 | O | HOH | X | 438 | 23.385 | 26.963 | 58.403 | 1.00 | 21.12 | O |
| ATOM | 3484 | O | HOH | X | 440 | 7.211 | 20.077 | 47.511 | 1.00 | 26.15 | O |
| ATOM | 3487 | O | HOH | X | 441 | 9.162 | 23.023 | 59.414 | 1.00 | 22.52 | O |
| ATOM | 3490 | O | HOH | X | 442 | 32.407 | 4.550 | 28.277 | 1.00 | 23.52 | O |
| ATOM | 3493 | O | HOH | X | 443 | 40.924 | 7.587 | 58.411 | 1.00 | 23.57 | O |
| ATOM | 3496 | O | HOH | X | 444 | 9.907 | 2.424 | 59.932 | 1.00 | 32.11 | O |
| ATOM | 3499 | O | HOH | X | 445 | 10.243 | 10.245 | 44.133 | 1.00 | 24.03 | O |
| ATOM | 3502 | O | HOH | X | 446 | 34.992 | 21.525 | 48.756 | 1.00 | 23.33 | O |
| ATOM | 3505 | O | HOH | X | 447 | 38.379 | 9.025 | 29.474 | 1.00 | 21.23 | O |
| ATOM | 3508 | O | HOH | X | 449 | 16.335 | 20.049 | 71.265 | 1.00 | 25.31 | O |
| ATOM | 3511 | O | HOH | X | 450 | 9.418 | 0.204 | 57.103 | 1.00 | 30.20 | O |
| ATOM | 3514 | O | HOH | X | 451 | 20.747 | 17.031 | 31.596 | 1.00 | 31.73 | O |
| ATOM | 3517 | O | HOH | X | 452 | 19.502 | −0.547 | 41.887 | 1.00 | 24.23 | O |
| ATOM | 3520 | O | HOH | X | 453 | 37.135 | 14.588 | 33.760 | 1.00 | 29.69 | O |
| ATOM | 3523 | O | HOH | X | 454 | 26.888 | 0.464 | 64.181 | 1.00 | 34.17 | O |
| ATOM | 3526 | O | HOH | X | 456 | 32.979 | −4.890 | 37.436 | 1.00 | 35.49 | O |
| ATOM | 3529 | O | HOH | X | 457 | 14.283 | 20.600 | 45.705 | 1.00 | 29.63 | O |
| ATOM | 3532 | O | HOH | X | 458 | 26.230 | 25.153 | 53.647 | 1.00 | 27.48 | O |
| ATOM | 3535 | O | HOH | X | 459 | 34.816 | 18.735 | 35.080 | 1.00 | 27.23 | O |
| ATOM | 3538 | O | HOH | X | 460 | 4.994 | 3.925 | 56.064 | 1.00 | 37.00 | O |
| ATOM | 3541 | O | HOH | X | 461 | 17.110 | 19.736 | 41.865 | 1.00 | 40.25 | O |
| ATOM | 3544 | O | HOH | X | 463 | 22.791 | −0.147 | 33.599 | 1.00 | 29.38 | O |
| ATOM | 3547 | O | HOH | X | 465 | 14.549 | −2.299 | 48.175 | 1.00 | 34.82 | O |
| ATOM | 3550 | O | HOH | X | 466 | 33.884 | 12.558 | 26.982 | 1.00 | 30.87 | O |
| ATOM | 3553 | O | HOH | X | 467 | 36.830 | 1.795 | 39.777 | 1.00 | 44.80 | O |
| ATOM | 3556 | O | HOH | X | 468 | 18.656 | 26.477 | 68.955 | 1.00 | 45.51 | O |
| ATOM | 3559 | O | HOH | X | 469 | 17.163 | 13.979 | 34.729 | 1.00 | 26.64 | O |
| ATOM | 3562 | O | HOH | X | 470 | 44.148 | 4.270 | 41.881 | 1.00 | 25.80 | O |
| ATOM | 3565 | O | HOH | X | 471 | 14.388 | 26.009 | 47.237 | 1.00 | 26.58 | O |
| ATOM | 3568 | O | HOH | X | 472 | 29.188 | 25.033 | 52.227 | 1.00 | 38.08 | O |
| ATOM | 3571 | O | HOH | X | 473 | 26.480 | 9.255 | 26.925 | 1.00 | 32.77 | O |
| ATOM | 3574 | O | HOH | X | 474 | 15.463 | 24.100 | 71.378 | 1.00 | 33.75 | O |
| ATOM | 3577 | O | HOH | X | 475 | 16.379 | 0.644 | 52.055 | 1.00 | 36.71 | O |
| ATOM | 3580 | O | HOH | X | 476 | 12.848 | −3.104 | 45.737 | 1.00 | 48.73 | O |
| ATOM | 3583 | O | HOH | X | 478 | 26.768 | 27.292 | 66.187 | 1.00 | 31.72 | O |
| ATOM | 3586 | O | HOH | X | 479 | 31.105 | 16.617 | 29.947 | 1.00 | 36.25 | O |
| ATOM | 3589 | O | HOH | X | 480 | 19.085 | 25.365 | 71.424 | 1.00 | 54.75 | O |
| ATOM | 3592 | O | HOH | X | 483 | 6.403 | 7.218 | 63.722 | 1.00 | 33.40 | O |
| ATOM | 3595 | O | HOH | X | 484 | 34.676 | −2.504 | 31.342 | 1.00 | 39.43 | O |
| ATOM | 3598 | O | HOH | X | 485 | 29.761 | 6.193 | 26.274 | 1.00 | 33.63 | O |
| ATOM | 3601 | O | HOH | X | 486 | 19.273 | −0.131 | 49.941 | 1.00 | 49.50 | O |
| ATOM | 3604 | O | HOH | X | 487 | 41.059 | −2.510 | 31.457 | 1.00 | 30.75 | O |
| ATOM | 3607 | O | HOH | X | 488 | 36.672 | −0.693 | 42.753 | 1.00 | 29.42 | O |
| ATOM | 3610 | O | HOH | X | 489 | 31.910 | −3.375 | 54.078 | 1.00 | 24.32 | O |
| ATOM | 3613 | O | HOH | X | 491 | 41.593 | 2.804 | 48.900 | 1.00 | 33.83 | O |
| ATOM | 3616 | O | HOH | X | 492 | 36.681 | 0.663 | 35.856 | 1.00 | 27.43 | O |
| ATOM | 3619 | O | HOH | X | 494 | 37.176 | 7.827 | 22.494 | 1.00 | 16.75 | O |
| ATOM | 3622 | O | HOH | X | 495 | 14.784 | 3.915 | 46.392 | 1.00 | 17.06 | O |
| ATOM | 3625 | O | HOH | X | 496 | 21.591 | 24.931 | 45.260 | 1.00 | 18.11 | O |
| ATOM | 3628 | O | HOH | X | 497 | 29.412 | 27.494 | 64.245 | 1.00 | 26.46 | O |
| ATOM | 3631 | O | HOH | X | 499 | 26.054 | 6.842 | 24.123 | 1.00 | 30.10 | O |
| ATOM | 3634 | O | HOH | X | 500 | 19.812 | 24.826 | 43.219 | 1.00 | 31.66 | O |
| ATOM | 3637 | O | HOH | X | 501 | 9.992 | −0.590 | 53.205 | 1.00 | 34.87 | O |
| ATOM | 3640 | O | HOH | X | 503 | 25.372 | 28.861 | 56.971 | 1.00 | 39.89 | O |
| ATOM | 3643 | O | HOH | X | 504 | 17.053 | 16.002 | 37.141 | 1.00 | 32.34 | O |
| ATOM | 3646 | O | HOH | X | 505 | 4.460 | 2.108 | 53.997 | 1.00 | 38.92 | O |
| ATOM | 3649 | O | HOH | X | 506 | 26.465 | 17.162 | 75.469 | 1.00 | 43.82 | O |
| ATOM | 3652 | O | HOH | X | 507 | 10.317 | 5.334 | 36.846 | 1.00 | 32.29 | O |
| ATOM | 3655 | O | HOH | X | 508 | 9.711 | 2.947 | 49.218 | 1.00 | 26.65 | O |
| ATOM | 3658 | O | HOH | X | 509 | 26.552 | −4.697 | 49.755 | 1.00 | 49.34 | O |
| ATOM | 3661 | O | HOH | X | 510 | 18.594 | 3.807 | 59.418 | 1.00 | 38.30 | O |
| ATOM | 3664 | O | HOH | X | 511 | 28.435 | −4.434 | 38.700 | 1.00 | 32.57 | O |
| ATOM | 3667 | O | HOH | X | 512 | 46.206 | 0.057 | 55.972 | 1.00 | 47.60 | O |
| ATOM | 3670 | O | HOH | X | 513 | 16.994 | −2.195 | 41.625 | 1.00 | 45.55 | O |
| ATOM | 3673 | O | HOH | X | 514 | 17.660 | 11.735 | 25.478 | 1.00 | 45.97 | O |
| ATOM | 3676 | O | HOH | X | 515 | 26.466 | 12.186 | 74.746 | 1.00 | 57.29 | O |
| ATOM | 3679 | O | HOH | X | 516 | 34.393 | 18.813 | 32.507 | 1.00 | 42.19 | O |
| ATOM | 3682 | O | HOH | X | 517 | 11.225 | 13.884 | 36.104 | 1.00 | 37.70 | O |
| ATOM | 3685 | O | HOH | X | 518 | 31.412 | −0.234 | 65.532 | 1.00 | 44.80 | O |
| ATOM | 3688 | O | HOH | X | 519 | 24.899 | 11.748 | 28.168 | 1.00 | 41.55 | O |
| ATOM | 3691 | O | HOH | X | 520 | 11.219 | 15.023 | 41.393 | 1.00 | 49.39 | O |
| ATOM | 3694 | O | HOH | X | 522 | 30.331 | 30.312 | 61.765 | 1.00 | 49.80 | O |
| ATOM | 3697 | O | HOH | X | 523 | 36.831 | −2.809 | 45.024 | 1.00 | 46.29 | O |
| ATOM | 3700 | O | HOH | X | 524 | 36.482 | 12.740 | 67.772 | 1.00 | 33.34 | O |
| ATOM | 3703 | O | HOH | X | 525 | 28.208 | 9.531 | 24.690 | 1.00 | 35.59 | O |
| ATOM | 3706 | O | HOH | X | 526 | 13.689 | 0.204 | 57.175 | 1.00 | 29.37 | O |
| ATOM | 3709 | O | HOH | X | 527 | 46.067 | 15.993 | 38.976 | 1.00 | 43.28 | O |
| ATOM | 3712 | O | HOH | X | 528 | 25.883 | −6.044 | 44.128 | 1.00 | 37.94 | O |
| ATOM | 3715 | O | HOH | X | 530 | 20.809 | 26.042 | 47.566 | 1.00 | 39.73 | O |
| ATOM | 3718 | O | HOH | X | 531 | 12.991 | 18.432 | 44.852 | 1.00 | 28.36 | O |
| ATOM | 3721 | O | HOH | X | 532 | 28.964 | −5.330 | 36.267 | 1.00 | 38.90 | O |
| ATOM | 3724 | O | HOH | X | 533 | 22.960 | 2.218 | 31.945 | 1.00 | 33.89 | O |
| ATOM | 3727 | O | HOH | X | 535 | 36.178 | 20.832 | 44.780 | 1.00 | 37.51 | O |
| ATOM | 3730 | O | HOH | X | 536 | 4.574 | 1.902 | 51.421 | 1.00 | 46.14 | O |
| ATOM | 3733 | O | HOH | X | 537 | 17.708 | 5.431 | 57.443 | 1.00 | 32.53 | O |
| ATOM | 3736 | O | HOH | X | 538 | 32.827 | 13.445 | 73.510 | 1.00 | 38.62 | O |
| ATOM | 3739 | O | HOH | X | 540 | 34.720 | −2.566 | 29.023 | 1.00 | 41.06 | O |
| ATOM | 3742 | O | HOH | X | 541 | 11.949 | −2.854 | 42.648 | 1.00 | 48.23 | O |
| ATOM | 3745 | O | HOH | X | 542 | 19.770 | −0.101 | 33.503 | 1.00 | 59.26 | O |
| ATOM | 3748 | O | HOH | X | 544 | 14.020 | 10.058 | 71.503 | 1.00 | 34.39 | O |
| ATOM | 3751 | O | HOH | X | 545 | 14.076 | 4.892 | 28.832 | 1.00 | 50.07 | O |
| ATOM | 3754 | O | HOH | X | 546 | 35.926 | 0.765 | 62.178 | 1.00 | 44.69 | O |
| ATOM | 3757 | O | HOH | X | 548 | 7.514 | 2.581 | 49.864 | 1.00 | 60.69 | O |
| ATOM | 3760 | O | HOH | X | 549 | 7.765 | 6.113 | 66.451 | 1.00 | 47.34 | O |
| ATOM | 3763 | O | HOH | X | 550 | 23.019 | 26.499 | 48.819 | 1.00 | 38.93 | O |
| ATOM | 3766 | O | HOH | X | 551 | 25.047 | −0.668 | 28.874 | 1.00 | 61.87 | O |
| ATOM | 3769 | O | HOH | X | 552 | 7.206 | −0.125 | 52.753 | 1.00 | 47.97 | O |
| ATOM | 3772 | O | HOH | X | 553 | 2.489 | 5.354 | 58.881 | 1.00 | 48.36 | O |
| ATOM | 3775 | O | HOH | X | 555 | 24.539 | −3.993 | 32.641 | 1.00 | 40.61 | O |
| ATOM | 3778 | O | HOH | X | 556 | 39.684 | −2.379 | 33.798 | 1.00 | 47.82 | O |
| ATOM | 3781 | O | HOH | X | 557 | 27.988 | 23.583 | 70.986 | 1.00 | 45.95 | O |
| ATOM | 3784 | O | HOH | X | 558 | 16.259 | 25.981 | 51.761 | 1.00 | 27.10 | O |
| ATOM | 3787 | O | HOH | X | 559 | 13.222 | −0.928 | 53.311 | 1.00 | 63.13 | O |
| ATOM | 3790 | O | HOH | X | 560 | 20.224 | 29.108 | 69.260 | 1.00 | 57.89 | O |
| ATOM | 3793 | O | HOH | X | 561 | 18.467 | 22.263 | 41.573 | 1.00 | 43.15 | O |
| ATOM | 3796 | S | SO4 | Y | 601 | 35.678 | 12.996 | 56.623 | 0.70 | 7.67 | S |
| ATOM | 3797 | O1 | SO4 | Y | 601 | 36.139 | 12.078 | 55.585 | 0.70 | 13.89 | O |
| ATOM | 3798 | O2 | SO4 | Y | 601 | 35.797 | 12.210 | 57.841 | 0.70 | 8.58 | O |
| ATOM | 3799 | O3 | SO4 | Y | 601 | 34.374 | 13.573 | 56.380 | 0.70 | 16.69 | O |
| ATOM | 3800 | O4 | SO4 | Y | 601 | 36.638 | 14.082 | 56.557 | 0.70 | 11.71 | O |
| ATOM | 3801 | S | SO4 | Y | 602 | 28.397 | 23.856 | 45.015 | 0.70 | 24.73 | O |
| ATOM | 3802 | O1 | SO4 | Y | 602 | 27.281 | 24.307 | 45.834 | 0.70 | 25.61 | O |
| ATOM | 3803 | O2 | SO4 | Y | 602 | 28.429 | 22.409 | 44.818 | 0.70 | 22.84 | O |
| ATOM | 3804 | O3 | SO4 | Y | 602 | 29.613 | 24.229 | 45.743 | 0.70 | 29.50 | O |
| ATOM | 3805 | O4 | SO4 | Y | 602 | 28.371 | 24.551 | 43.749 | 0.70 | 28.60 | O |
| ATOM | 3806 | S | SO4 | Y | 603 | 42.900 | 20.990 | 81.966 | 0.70 | 29.41 | O |
| ATOM | 3807 | O1 | SO4 | Y | 603 | 43.188 | 21.619 | 80.689 | 0.70 | 23.17 | O |
| ATOM | 3808 | O2 | SO4 | Y | 603 | 43.931 | 20.109 | 82.488 | 0.70 | 30.62 | O |
| ATOM | 3809 | O3 | SO4 | Y | 603 | 41.665 | 20.213 | 81.898 | 0.70 | 31.55 | O |
| ATOM | 3810 | O4 | SO4 | Y | 603 | 42.726 | 22.118 | 82.860 | 0.70 | 32.81 | O |
| ATOM | 3811 | S | SO4 | Y | 604 | 34.328 | 18.651 | 77.072 | 0.70 | 36.68 | S |
| ATOM | 3812 | O1 | SO4 | Y | 604 | 33.645 | 17.961 | 75.979 | 0.70 | 36.62 | O |
| ATOM | 3813 | O2 | SO4 | Y | 604 | 34.229 | 17.813 | 78.264 | 0.70 | 37.58 | O |
| ATOM | 3814 | O3 | SO4 | Y | 604 | 33.640 | 19.910 | 77.283 | 0.70 | 35.92 | O |
| ATOM | 3815 | O4 | SO4 | Y | 604 | 35.749 | 18.899 | 76.842 | 0.70 | 32.21 | O |
| ATOM | 3816 | S | SO4 | Y | 605 | 20.767 | −0.638 | 38.261 | 0.70 | 22.29 | S |
| ATOM | 3817 | O1 | SO4 | Y | 605 | 21.835 | −1.312 | 37.474 | 0.70 | 17.77 | O |
| ATOM | 3818 | O2 | SO4 | Y | 605 | 20.750 | 0.822 | 37.951 | 0.70 | 20.00 | O |
| ATOM | 3819 | O3 | SO4 | Y | 605 | 20.968 | −1.030 | 39.620 | 0.70 | 20.92 | O |
| ATOM | 3820 | O4 | SO4 | Y | 605 | 19.500 | −1.224 | 37.828 | 0.70 | 28.20 | O |
| ATOM | 3821 | S | SO4 | Y | 606 | 29.483 | −6.067 | 47.771 | 0.70 | 27.74 | S |
| ATOM | 3822 | O1 | SO4 | Y | 606 | 30.669 | −5.901 | 46.925 | 0.70 | 29.74 | O |
| ATOM | 3823 | O2 | SO4 | Y | 606 | 29.713 | −7.052 | 48.825 | 0.70 | 30.93 | O |
| ATOM | 3824 | O3 | SO4 | Y | 606 | 28.393 | −6.449 | 46.875 | 0.70 | 29.72 | O |
| ATOM | 3825 | O4 | SO4 | Y | 606 | 29.111 | −4.839 | 48.457 | 0.70 | 18.37 | O |
| ATOM | 3826 | S | SO4 | Y | 607 | 39.989 | 16.559 | 37.193 | 0.70 | 24.82 | S |
| ATOM | 3827 | O1 | SO4 | Y | 607 | 39.493 | 17.029 | 35.901 | 0.70 | 31.09 | O |
| ATOM | 3828 | O2 | SO4 | Y | 607 | 40.305 | 15.142 | 37.088 | 0.70 | 26.22 | O |
| ATOM | 3829 | O3 | SO4 | Y | 607 | 38.909 | 16.710 | 38.115 | 0.70 | 24.47 | O |
| ATOM | 3830 | O4 | SO4 | Y | 607 | 41.271 | 17.168 | 37.535 | 0.70 | 23.92 | O |
| ATOM | 3831 | S | SO4 | Y | 608 | 31.245 | 20.872 | 79.064 | 0.70 | 129.03 | S |
| ATOM | 3832 | O1 | SO4 | Y | 608 | 30.482 | 20.410 | 77.908 | 0.70 | 128.90 | O |
| ATOM | 3833 | O2 | SO4 | Y | 608 | 31.998 | 19.756 | 79.631 | 0.70 | 129.11 | O |
| ATOM | 3834 | O3 | SO4 | Y | 608 | 30.330 | 21.401 | 80.071 | 0.70 | 129.13 | O |
| ATOM | 3835 | O4 | SO4 | Y | 608 | 32.164 | 21.927 | 78.647 | 0.70 | 129.06 | O |
| ATOM | 3836 | O3 | GOL | A | 1 | 20.441 | 14.331 | 40.631 | 1.00 | 11.20 | O |
| ATOM | 3838 | C3 | GOL | A | 1 | 20.040 | 15.547 | 40.073 | 1.00 | 11.44 | C |
| ATOM | 3841 | C2 | GOL | A | 1 | 19.516 | 16.587 | 41.042 | 1.00 | 13.43 | C |
| ATOM | 3843 | O2 | GOL | A | 1 | 20.554 | 17.176 | 41.804 | 1.00 | 14.62 | O |
| ATOM | 3845 | C1 | GOL | A | 1 | 18.490 | 16.011 | 42.030 | 1.00 | 14.51 | C |
| ATOM | 3848 | O1 | GOL | A | 1 | 17.955 | 17.070 | 42.775 | 1.00 | 17.23 | O |
| ATOM | 3850 | O3 | GOL | A | 2 | 37.747 | 20.476 | 54.065 | 1.00 | 40.41 | O |
| ATOM | 3852 | C3 | GOL | A | 2 | 37.169 | 19.407 | 53.339 | 1.00 | 36.60 | C |
| ATOM | 3855 | C2 | GOL | A | 2 | 38.227 | 18.479 | 52.776 | 1.00 | 32.19 | C |
| ATOM | 3857 | O2 | GOL | A | 2 | 38.442 | 17.656 | 53.815 | 1.00 | 26.25 | O |
| ATOM | 3859 | C1 | GOL | A | 2 | 39.572 | 19.037 | 52.365 | 1.00 | 29.78 | C |
| ATOM | 3862 | O1 | GOL | A | 2 | 40.418 | 18.008 | 51.922 | 1.00 | 38.17 | O |

TABLE 1A

IC₅₀ values of peptides and ephrin-B2 for
inhibition of mouse ephrin-B2 alkaline
phosphatase (AP) binding to immobilized
mouse EphB4 ectodomain Fc fusion protein
using ELISA binding assays.

| | |
|---|---|
| Inhibitor | IC₅₀ |

| | |
|---|---|
| ephrin-B2 Fc | 9 nM |
| Human ephrin-B2 monomer | 10 nM |
| TNYLFSPNGPIARAW | 15 nM |
| (TNYL-RAW; SEQ ID NO:1)* | |
| -NYLFSPNGPIARAW | 40 nM |
| (NYLF-RAW; SEQ ID NO:30) | |
| --YLFSPNGPIARAW | 40 nM |
| (YLFS-RAW; SEQ ID NO:31) | |
| ---LFSPNGPIARAW | >10 μM |
| (LFSP-RAW; SEQ ID NO:32) | |
| TNYLFSPNGPIA | 150 μM |
| (TNYL; SEQ ID NO:33) | |
| TNYLFSPNGPIAGSGSK-biotin | 50 μM |
| (TNYL-biotin; SEQ ID NO:34) | |

*Ephrin-B2 G-H loop: KFQEFSPNLWGLEFQK (SEQ ID NO:35)

TABLE 1B
Binding of peptides and human ephrin-B2
to human EphB4 ephrin-binding domain.

| Ligand | K_{d} (nM)* | ΔG (kcal mol⁻¹) | ΔH (kcal mol⁻¹) | TΔS (kcal mol⁻¹) |
|---|---|---|---|---|
| TNYL-RAW | 71 ± 14 | −9.8 ± 0.1 | −14.7 ± 0.2 | −4.9 ± 0.2 |
| NYLF-RAW | 65 ± 7 | −9.8 ± 0.1 | −15.5 ± 0.1 | −5.7 ± 0.1 |
| YLFS-RAW | 80 ± 36 | −9.7 ± 0.2 | −13.8 ± 0.5 | −4.1 ± 0.4 |
| LFSP-RAW | 3,500 ± 680 | −7.4 ± 0.1 | −5.3 ± 0.5 | 2.1 ± 0.4 |
| TNYL | ≧140,000 | ND | −9.6 ± 0.3 | ND |
| ephrin-B2 | 40 ± 20 | −10.2 ± 0.3 | 3.3 ± 0.1 | 13.4 ± 0.4 |

Experiments were performed at 25° C. in 50 mM Tris pH 7.8, 150 mM NaCl, 1 mM CaCl₂. All values (except for TNYL) represent the average of at least two experiments.
*The K_{d} value for the TNYL peptide is a lower limit assuming a stoichiometry of 1 and at least 70% saturation of binding at a final peptide concentration of 300 μM.

TABLE 2
Crystallographic Statistics for the EphB4-TNYL-RAW Complex

| | |
|---|---|
| Resolution (Å)¹ | 40-1.65 (1.71-1.65) |
| Space Group | P4₁2₁2 |
| Unit Cell Dimensions (Å) | a = b = 60.97, c = 151.7 |
| Completeness (%) | 100 (99.7) |
| R_{sym} (%)² | 3.9 (20.8) |
| I/σ | 42.7 (7.2) |
| Mean Redundancy | 3.7 (3.2) |
| No. Reflections | 32,786 |
| R_{cryst} (%)³ | 16.0 (17.4) |
| R_{free} (%)⁴ | 19.1 (20.2) |
| R.m.s. deviations | |
| Bond length (Å) | 0.02 |
| Bond angle (°) | 1.7 |
| Improper (°) | 1.4 |
| Number of atoms | |
| Protein | 1486 |
| Solvent | 214 |
| Peptide | 115 |
| Sulfate | 8 |
| Glycerol | 3 |

¹Number in parentheses is for the highest shell.
²R_{sym} = |I I|/I, where I is the observed intensity and I is the average intensity of multiple symmetry-related observations of that reflection.
³R_{cryst} = F_{obs}| |F_{calc}/|F_{obs}|, where F_{obs} and F_{calc} are the observed and calculated structure factors. R_{sym} = |I I|/I, where I is the observed intensity and I is the average intensity of multiple symmetry-related observations of that reflection.
⁴R_{free} = F_{obs}| |F_{calc}/|F_{obs}| for 10% of the data not used at any stage of structural refinement.

### EXAMPLES

Aspects of the present teachings may be further understood in light of the following examples, which should not be construed as limiting the scope of the present teachings in any way.

### Example 1

Construct design, expression and purification of EphB4: Twelve sequential 4 amino acid truncations in human EphB4 were designed based on EphB4-EphB2 sequence alignment in the region C-terminal to the last β-strand in the EphB2 structure. The resulting fragments were cloned into the insect cell expression vector pBAC6 (Novagen, WI) under control of the heterologous GP64 signal peptide and containing a N-terminal six histidine tag. Constructs were sequence verified, and baculovirus was generated using homologous recombination into Sapphire Baculovirus DNA (Orbigen, CA) using the manufacturers protocol. After 3 rounds of viral amplification, a small scale expression screen was conducted for all constructs in both Sf9 and Hi5 insect cells. Briefly, 5E10+6 cells were infected with baculovirus at an MOI of 2 in 38 mm tissue culture dishes; cells were harvested at 48 hours post infection and supernatant containing secreted EphB4 was concentrated 10-fold and buffer exchanged into 50 mM Tris pH 7.8, 400 mM NaCl, and 5 mM imidazole using an Amicon Ultra 5K concentrator (Millipore, MA). The secreted protein was bound to Ni-NTA magnetic beads (Qiagen, CA), washed with 50 mM Tris pH7.8, 400 mM NaCl, 20 mM Imidazole buffer and eluted with 50 mM Tris pH 7.8, 400 mM NaCl, 250 mM Imidazole. Based on analysis of immobilized metal affinity chromatography (IMAC) elutes, the EphB4 (17-196) construct was identified as the highest expressor at ˜6 mg/L in Hi5 insect cells. Large scale expression was conducted using Wave Bioreactors(Wave Biotech LLC, NJ) at a MOI of 2 for 48 hours in Hi5 insect cells. Media containing secreted EphB4 was concentrated and buffer exchanged using a Hydrosart Crossflow filter (Sartorius, NY). Following IMAC purification on ProBond resin (Invitrogen, CA) as described above, EphB4 was concentrated to 5 mg/ml and loaded on a Superdex 75 16/60 column (GE HealthCare, NY). A small amount of aggregated material was removed by preparative size exclusion chromatography, while most of the sample eluted in a single peak corresponding to an EphB4 (17-196) monomer. The complete removal of the GP64secretion sequence and protein identity were confirmed by MALDI analysis.

### Example 2

Crystallization: Purified EphB4 was concentrated to 10 mg/mL in 25 mM Tris, pH 7.8, 150 mM NaCl, and 5 mM CaCl2 in the presence of a 3-fold molar excess of TNYL-RAW peptide (SEQ ID NO: 1; Biopeptide, Inc.). The EphB4 17-196 construct was crystallized by sitting drop vapor diffusion at 20° C. against a reservoir of 2.2 M ammonium sulfate and 200 mM NaCl, and cryoprotected in 25% glycerol.

### Example 3

Structure Determination: Crystals of the EphB4-TNYL complex grew in the P41212 space group (a=60.92, c=151.93). A single crystal diffracted to 1.65 Å resolution at 100 K on beamline 5-1 at the Advanced Light Source (Berkeley, Calif.), and were integrated, reduced and scaled using HKL2000 (Otwinowski, 1997). The structure was determined by molecular replacement with MolRep (CCP4i) (CCP4, 1994; Vagin, 1997) using the structure of apo EphB2 (pdb id:1NUK (Himanen et al., 1998)) as a search model. The structure was refined with CNS using torsion angle dynamics and the maximum likelihood function target (Table 2), and manual model building performed with the program O (Bringer et al., 1998; Jones et al., 1991). Electron density for the TNYL-RAW peptide was clear after the first round of refinement, with the positioning of the critical RAW sequence clearly evident in the initial |Fobs|−|Fcalc| maps (FIG. 2). The peptide was initially built as a polyalanine chain, while unbiased electron density for the peptide from simulated annealing omit maps was used to build the full peptide. Residues 17-196 from EphB4, and 14 of 15 residues from the TNYL-RAW peptide, could be readily traced into electron density. The final structure exhibits good geometry with no Ramachandran outliers. Figures were created with PyMol, Molscript, Raster3D, Dino, and Povray (DeLano, 2002; Esnouf, 1997;Kraulis, 1991; Merritt and Murphy, 1994).

### Example 4

Isothermal titration calorimetry and ELISA experiments: EphB4and ephrin-B2 were either dialyzed or buffer exchanged into 50 mM Tris-Cl (pH 7.8 at 25° C.), 150 mM NaCl, 1 mM CaCl₂, prior to use in calorimetry experiments. Peptides were dissolved into the same buffer used for the dialysis of EphB4. The concentration of EphB4, ephrin-B2 and the peptides was determined by measuring the A₂₈₀ and using the theoretical-extinction coefficient (Gill and von Hippel, 1989). ITC experiments were performed with a Microcal MCS ITC at 25° C. Following an initial injection of 2 μl, titrations were performed by making 20 13 μl injections of peptide into EphB4 in the sample cell to produce an approximate final 2:1 ratio of injectant to sample in the cell. For most titrations the sample cell contained 15 μM EphB4 and the injection syringe contained a 200 μM solution of the peptide. Titrations with ephrin-B2 contained 13 μM EphB4 in the sample cell and 290 μM ephrin-B2 in the syringe. Prior to loading the sample cell, EphB4 was centrifuged at 18,000 g for 5 min at 4° C. to remove aggregates and degassed for 5 minutes at room temperature. Corrections for heats of dilution for the peptides and ephrin-B2 were determined by performing titrations of peptide or ephrin-B2 solutions into buffer. Dilution data were fit to a line and subtracted from the corresponding titration data. Titration data were analyzed using Origin ITC software (Version 5.0, Microcal Software Inc.) and curves were fit to a single binding site model (Wiseman et al., 1989). The low affinity of the TNYL peptide and the limited availability of EphB4 (17-196) precluded accurate determination of the K_{d} for this interaction by ITC. A lower limit for the binding constant was determined by performing a titration in which the sample cell contained 30 μM EphB4 and the injection syringe contained a 1.45 mM solution of the peptide, producing a final ratio of peptide to EphB4 of 10:1. The data was fit assuming a stoichiometry of 1 and at least 60% saturation of binding at the final peptide concentration (Turnbull and Daranas, 2003).

The ability of peptides to compete the binding of mouse ephrin-B2 alkaline phosphatase to immobilized mouse EphB4-Fc-His (R&D Systems) was measured by ELISA as previously described (Koolpe et al., 2005).

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Publications incorporated herein by reference in their entirety include:

Adams, R. H., Wilkinson, G. A., Weiss, C., Diella, F., Gale, N. W., Deutsch, U., Risau, W., and Klein, R. (1999). Roles of ephrin-B ligands and EphB receptors in cardiovascular development: demarcation of arterial/venous domains, vascular morphogenesis, and sprouting angiogenesis. Genes Dev 13, 295-306.

Brantley-Sieders, D. M., and Chen, J. (2004). Eph receptor tyrosine kinases in angiogenesis: from development to disease. Angiogenesis 7, 17-28.

Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., et al. (1998). Crystallography & NMR system: A new software suite for macromolecular structure determination. Acta Crystallogr D Biol Crystallogr 54 (Pt 5), 905-921.

Carmeliet, P., and Collen, D. (1999). Role of vascular endothelial growth factor and vascular endothelial growth factor receptors in vascular development. Curr Top Microbiol Immunol 237, 133-158.

CCP4 (1994). The CCP4 suite: Programs for X-ray crystallography. Acta Crystallogr D Biol Crystallogr 50, 760-763.

Cheng, N., Brantley, D. M., and Chen, J. (2002). The ephrins and Eph receptors in angiogenesis. Cytokine Growth Factor Rev 13, 75-85.

DeLano, W. L. (2002). The PyMOL molecular graphics system. world wide web.

Dodelet, V. C., and Pasquale, E. B. (2000). Eph receptors and ephrin ligands: embryogenesis to tumorigenesis. Oncogene 19, 5614-5619.

Eph Nomenclature Committee (1997). Unified nomenclature for Eph family receptors and their ligands, the ephrins. Cell 90, 403-404.

Esnouf, R. M. (1997). An extensively modified version of MolScript that includes greatly enhanced coloring capabilities. J Mol Graph Model 15, 132-134, 112-133.

Ferrara, N. (1999). Vascular endothelial growth factor: molecular and biological aspects. Curr Top Microbiol Immunol 237, 1-30.

Fuller, T., Korff, T., Kilian, A., Dandekar, G., and Augustin, H. G. (2003). Forward EphB4 signaling in endothelial cells controls cellular repulsion and segregation from ephrinB2 positive cells. J Cell Sci 116, 2461-2470.

Gale, N. W., Holland, S. J., Valenzuela, D. M., Flenniken, A., Pan, L., Ryan, T. E., Henkemeyer, M., Strebhardt, K., Hirai, H., Wilkinson, D. G., et al. (1996). Eph receptors and ligands comprise two major specificity subclasses and are reciprocally compartmentalized during embryogenesis. Neuron 17, 9-19.

Gale, N. W., and Yancopoulos, G. D. (1999). Growth factors acting via endothelial cell-specific receptor tyrosine kinases: VEGFs, angiopoietins, and ephrins in vascular development. Genes Dev 13, 1055-1066.

Gerety, S. S., Wang, H. U., Chen, Z. F., and Anderson, D. J. (1999). Symmetrical mutant phenotypes of the receptor EphB4 and its specific transmembrane ligand ephrin-B2 in cardiovascular development. Mol Cell 4, 403-414.

Gill, S. C., and von Hippel, P. H. (1989). Calculation of protein extinction coefficients from amino acid sequence data. Anal Biochem 182, 319-326.

Hamada, K., Oike, Y., Ito, Y., Maekawa, H., Miyata, K., Shimomura, T., and Suda, T. (2003). Distinct roles of ephrin-B2 forward and EphB4 reverse signaling in endothelial cells. Arterioscler Thromb Vasc Biol 23, 190-197.

Himanen, J. P., Chumley, M. J., Lackmann, M., Li, C., Barton, W. A., Jeffrey, P. D., Vearing, C., Geleick, D., Feldheim, D. A., Boyd, A. W., et al. (2004). Repelling class discrimination: ephrin-A5 binds to and activates EphB2 receptor signaling. Nat Neurosci 7, 501-509.

Himanen, J. P., Henkemeyer, M., and Nikolov, D. B. (1998). Crystal structure of the ligand-binding domain of the receptor tyrosine kinase EphB2. Nature 396, 486-491.

Himanen, J. P., Rajashankar, K. R., Lackmann, M., Cowan, C. A., Henkemeyer, M., and Nikolov, D. B. (2001). Crystal structure of an Eph receptor-ephrin complex. Nature 414, 933-938.

cones, T. A., Zou, J. Y., Cowan, S. W., and Kjeldgaard (1991). Improved methods for building protein models in electron density maps and the location of errors in these models. Acta Crystallogr A 47 (Pt 2), 110-119.

Koolpe, M., Burgess, R., Dail, M., and Pasquale, E. B. (2005). EphB receptor-binding peptides identified by phage display enable design of an antagonist with ephrin-like affinity. J Biol Chem 280, 17301-17311.

Koolpe, M., Dail, M., and Pasquale, E. B. (2002). An ephrin mimetic peptide that selectively targets the EphA2 receptor. J Biol Chem 277, 46974-46979.

Kraulis, P. J. (1991). MOLSCRIPT: a program to produce both detailed and schematic plots of protein structures. J Applied Crystallography 24, 946-950.

Kullander, K., and Klein, R. (2002). Mechanisms and functions of Eph and ephrin signalling. Nat Rev Mol Cell Biol 3, 475-486.

Martiny-Baron, G., Korff, T., Schaffner, F., Esser, N., Eggstein, S., Marme, D., and Augustin, H. G. (2004). Inhibition of tumor growth and angiogenesis by soluble EphB4. Neoplasia 6, 248-257.

Merritt, E. A., and Murphy, M. E. (1994). Raster3D Version 2.0. A program for photorealistic molecular graphics. Acta Crystallogr D Biol Crystallogr 50, 869-873.

Murai, K. K., Nguyen, L. N., Koolpe, M., McLennan, R., Krull, C. E., and Pasquale, E. B. (2003). Targeting the EphA4 receptor in the nervous system with biologically active peptides. Mol Cell Neurosci 24, 1000-1011.

Nikolov, D. B., Li, C., Barton, W. A., and Himanen, J. P. (2005). Crystal structure of the ephrin-b1 ectodomain: implications for receptor recognition and signaling. Biochemistry 44, 10947-10953.

Noren, N. K., Lu, M., Freeman, A. L., Koolpe, M., and Pasquale, E. B. (2004). Interplay between EphB4 on tumor cells and vascular ephrin-B2 regulates tumor growth. Proc Natl Acad Sci USA 101, 5583-5588.

Otwinowski, Z., Minor, W. (1997). Processing of x-ray diffraction data collected in oscillation mode, Vol 276 (New York, Academic Press).

Pasquale, E. B. (2005). Eph receptor signalling casts a wide net on cell behaviour. Nat Rev Mol Cell Biol 6, 462-475.

Smith, F. M., Vearing, C., Lackmann, M., Treutlein, H., Himanen, J., Chen, K., Saul, A., Nikolov, D., and Boyd, A. W. (2004). Dissecting the EphA3/Ephrin-A5 interactions using a novel functional mutagenesis screen. J Biol Chem 279, 9522-9531.

Stephenson, S. A., Slomka, S., Douglas, E. L., Hewett, P. J., and Hardingham, J. E. (2001). Receptor protein tyrosine kinase EphB4 is up-regulated in colon cancer. BMC Mol Biol 2, 15.

Takemoto, M., Fukuda, T., Sonoda, R., Murakami, F., Tanaka, H., and Yamamoto, N. (2002). Ephrin-B3-EphA4 interactions regulate the growth of specific thalamocortical axon populations in vitro. Eur J Neurosci 16, 1168-1172.

Turnbull, W. B., and Daranas, A. H. (2003). On the value of c: can low affinity systems be studied by isothermal titration calorimetry? J Am Chem Soc 125, 14859-14866.

Vagin, A., Teplyakov, A. (1997). MOLREP: an automated program for molecular replacement. J Appl Crystallogr 30, 1022-1025.

Wang, H. U., Chen, Z. F., and Anderson, D. J. (1998). Molecular distinction and angiogenic interaction between embryonic arteries and veins revealed by ephrin-B2 and its receptor Eph-B4. Cell 93, 741-753.

Wilkinson, D. G. (2000). Eph receptors and ephrins: regulators of guidance and assembly. Int Rev Cytol 196, 177-244.

Wiseman, T., Williston, S., Brandts, J. F., and Lin, L. N. (1989). Rapid measurement of binding constants and heats of binding using a new titration calorimeter. Anal Biochem 179, 131-137.

Xia, G., Kumar, S. R., Masood, R., Zhu, S., Reddy, R., Krasnoperov, V., Quinn, D. I., Henshall, S. M., Sutherland, R. L., Pinski, J. K., et al. (2005). EphB4 expression and biological significance in prostate cancer. Cancer Res 65, 4623-4632.

Yuan, K., Hong, T. M., Chen, J. J., Tsai, W. H., and Lin, M. T. (2004). Syndecan-1 up-regulated by ephrinB2/EphB4 plays dual roles in inflammatory angiogenesis. Blood 104, 1025-1033.

Zamora, D. O., Davies, M. H., Planck, S. R., Rosenbaum, J. T., and Powers, M. R. (2005). Soluble forms of EphrinB2 and EphB4 reduce retinal neovascularization in a model of proliferative retinopathy. Invest Ophthalmol Vis Sci 46, 2175-2182.

What is claimed is:

1. A method for designing a drug which interferes with an activity of an EphB4 receptor, the method comprising:
(a) providing on a digital computer a three-dimensional structure of a receptor-ligand complex comprising the EphB4 receptor and at least one ligand of the EphB4 receptor; and
(b) using software comprised by the digital computer to design a chemical compound which is predicted to bind to the EphB4 receptor.

2. A method according to claim 1, further comprising:
(c) synthesizing the chemical compound; and
(d) evaluating the chemical compound for an ability to interfere with an activity of the EphB4 receptor.

3. A method according to claim 1, wherein the chemical compound is designed by computational interaction with reference to a three-dimensional site of the structure of the receptor-ligand complex, wherein the three-dimensional site is selected from the group consisting of EphB4 D-E and J-K loops.

4. A method according to claim 3, wherein the three-dimensional site comprises Leu-48, Cys-61, Leu-95, Ser-99 Leu-100, Pro-101, Thr-147, Lys-149, Ala-155, and Cys-184 of SEQ ID NO: 27.

5. A method according to claim 1, wherein the EphB4 receptor is a human EphB4 receptor.

6. A method for determining a three-dimensional structure of a target EphB receptor-ligand complex structure comprising:
(a) providing an amino acid sequence of a target EphB structure, wherein the three-dimensional structure of the target EphB structure is not known;
(b) predicting a pattern of folding of the amino acid sequence in a three-dimensional conformation using a fold recognition algorithm; and
(c) comparing the pattern of folding of the target structure amino acid sequence with the three-dimensional structure of a known EphB4 receptor-ligand complex.

7. A method in accordance with claim 6, wherein the EphB4 receptor comprises an amino acid sequence as set forth in SEQ ID NOs: 2 or 3.

8. A method in accordance with claim 6, wherein the EphB4 receptor consists essentially of an amino acid sequence as set forth in SEQ ID NOs: 2 or 3.

9. A method in accordance with claim 7, wherein the known receptor-ligand complex comprises a three-dimensional structure described by atomic coordinates that substantially conform to atomic coordinates set forth in Table 1.

10. A method according to claim 6, wherein the EphB4 receptor is a human EphB4 receptor.

11. A method for generating a model of a three-dimensional structure of an EphB ligand complex, the method comprising:
(a) providing an amino acid sequence of a reference EphB4 polypeptide and an amino acid sequence of a target EphB comprised by the EphB-ligand complex;
(b) identifying structurally conserved regions shared between the reference EphB4 amino acid sequence and the target EphB amino acid sequence; and
(c) assigning atomic coordinates from the conserved regions to the target EphB ligand complex.

12. A method in accordance with claim 11, wherein the EphB4 polypeptide comprises an amino acid sequence as set forth in SEQ ID NOs: 2 or 3.

13. A method in accordance with claim 11, wherein the EphB4 polypeptide consists essentially of an amino acid sequence as set forth in SEQ ID NOs: 2 or 3.

14. A method in accordance with claim 11, wherein the target EphB-ligand complex comprises a three-dimensional structure described by atomic coordinates that substantially conform to atomic coordinates set forth in Table 1.

15. A method in accordance with claim 11, wherein the reference EphB4-ligand complex comprises a three-dimensional structure described by atomic coordinates that substantially conform to atomic coordinates set forth in Table 1.

16. A method according to claim 11, wherein the EphB4 polypeptide is a human EphB4 polypeptide.

17. A method for generating a model of a three-dimensional structure of an EphB receptor-ligand complex, the method comprising:
(a) providing an amino acid sequence of a known EphB4 receptor in complex with at least one known ligand of the EphB4 receptor;
(b) providing an amino acid sequence of a target EphB receptor in complex with at least one target ligand of the EphB receptor;
(c) identifying structurally conserved regions shared between the known receptor ligand complex amino acid sequence and the target receptor-ligand complex amino acid sequence; and
(d) assigning atomic coordinates of the conserved regions to the target receptor ligand complex.

18. A method in accordance with claim 17, wherein the EphB4 receptor comprises an amino acid sequence as set forth in SEQ ID NOs: 2 or 3.

19. A method in accordance with claim 17, wherein the EphB4 receptor consists essentially of an amino acid sequence as set forth in SEQ ID NOs: 2 or 3.

20. A method according to claim 17, wherein the EphB4 receptor is a human EphB4 receptor.

21. A method according to claim 17, wherein the known receptor-ligand complex comprises a three-dimensional structure described by atomic coordinates that substantially conform to Table 1.

22. A crystal comprising an EphB4 ligand binding domain and a ligand.

23. A crystal according to claim 22, wherein the EphB4 ligand binding domain is a polypeptide having a sequence of SEQ ID NOs: 2 or 3.

24. A crystal according to claim 22, wherein the EphB4 ligand binding domain consists essentially of EphB4 D-E and J-K loops.

25. A crystal according to claim 22, wherein the EphB4 ligand binding domain consists essentially of Leu-48, Cys-61, Leu-95, Ser-99 Leu-100, Pro-101, Thr-147, Lys-149, Ala-155, and Cys-184 of SEQ ID NO: 27.

26. A crystal according to claim 22, wherein the EphB4 ligand binding domain is a human EphB4 ligand binding domain.

27. A crystal in accordance with claim 22, wherein the ligand is ephrin-B2.

28. A crystal according to claim 22, wherein the ligand comprises Phe-120, Pro-122, Leu-124, Trp-125, and Leu-127 of ephrin-B2.

29. A crystal according to claim 22, wherein the ligand comprises sequence motif NxWxL, wherein x is any amino acid.

30. A crystal in accordance with claim 22, wherein the ligand is a polypeptide having SEQ ID NO: 1.

31. A crystal according to claim 22, wherein the ligand is a polypeptide elected from the group consisting of polypeptides having SEQ ID NO: 4 through SEQ ID NO: 26.

32. A crystal according to claim 22, wherein the ligand is a polypeptide having at least 50% sequence identity to a polypeptide selected from the group consisting of polypeptides having SEQ ID NO: 1 and SEQ ID NO: 4 through SEQ ID NO: 26.

33. A crystal according to claim 22, wherein the ligand is a polypeptide having at least 75% sequence identity to a polypeptide selected from the group consisting of polypeptides having SEQ ID NO: 1 and SEQ ID NO: 4 through SEQ ID NO: 26.

34. A crystal according to claim 22, wherein the ligand is a polypeptide having at least 90% sequence identity to a polypeptide selected from the group consisting of polypeptides having SEQ ID NO: 1 and SEQ ID NO: 4 through SEQ ID NO: 26.

35. A crystal in accordance with claim 22, wherein the crystal comprises space group P41212 so as to form a unit cell of dimensions a=60.97 Å, b=60.97 Å, and c=151.7 Å.

36. A crystal comprising a polypeptide having SEQ ID NOs: 2 or 3 complexed with a ligand, wherein the crystal is sufficiently pure to determine atomic coordinates of the complex by X-ray diffraction to a resolution of about 1.65 Å.

37. A crystal according to claim 36, wherein the ligand comprises Phe-120, ProDocket 122, Leu-124, Trp-125, and Leu-127 of ephrin-B2.

38. A crystal according to claim 36, wherein the ligand comprises sequence motif NxWxL, wherein x is any amino acid.

39. A crystal in accordance with claim 36, wherein the ligand is a polypeptide having SEQ ID NO: 1.

40. A crystal according to claim 36, wherein the ligand is a polypeptide selected from the group consisting of polypeptides having SEQ ID NO: 4 through SEQ ID NO: 26.

41. A crystal according to claim 36, wherein the ligand is a polypeptide having at least 50% sequence identity to a polypeptide selected from the group consisting of polypeptides having SEQ ID NO: 1 and SEQ ID NO: 4 through SEQ ID NO: 26.

42. A crystal according to claim 36, wherein the ligand is a polypeptide having at least 75% sequence identity to a polypeptide selected from the group consisting of polypeptides having SEQ ID NO: 1 and SEQ ID NO: 4 through SEQ ID NO: 26.

43. A crystal according to claim 36, wherein the ligand is a polypeptide having at least 90% sequence identity to a polypeptide selected from the group consisting of polypeptides having SEQ ID NO: 1 and SEQ ID NO: 4 through SEQ ID NO: 26.

44. A polypeptide having SEQ ID NOs: 2 or 3 in complex with a ligand.

45. A complex according to claim 44, wherein the ligand is an ephrin.

46. A complex according to claim 45, wherein the ephrin is ephrin-B2.

47. A complex according to claim 44, wherein the ligand comprises Phe-120, Pro-122, Leu-124, Trp-125, and Leu-127 of ephrin-B2.

48. A complex according to claim 44, wherein the ligand comprises sequence motif NxWxL, wherein x is any amino acid.

49. A complex according to claim 44, wherein the ligand is a polypeptide having SEQ ID NO: 1.

50. A complex according to claim 44, wherein the ligand is a polypeptide selected from the group consisting of polypeptides having SEQ ID NO: 4 through SEQ ID NO: 26.

51. A complex according to claim 44, wherein the ligand is a polypeptide having at least 50% sequence identity to a polypeptide selected from the group consisting of polypeptides having SEQ ID NO: 1 and SEQ ID NO: 4 through SEQ ID NO: 26.

52. A complex according to claim 44, wherein the ligand is a polypeptide having at least 75% sequence identity to a polypeptide selected from the group consisting of polypeptides having SEQ ID NO: 1 and SEQ ID NO: 4 through SEQ ID NO: 26.

53. A complex according to claim 44, wherein the ligand is a polypeptide having at least 90% sequence identity to a polypeptide selected from the group consisting of polypeptides having SEQ ID NO: 1 and SEQ ID NO: 4 through SEQ ID NO: 26.

54. A therapeutic compound that inhibits an activity of an EphB4 receptor, wherein the compound is selected by
a) performing a structure based drug design using a three-dimensional structure determined for a crystal comprising an EphB4 receptor and a ligand;
b) contacting a sample comprising the EphB4 receptor with the compound, and
c) detecting inhibition of at least one activity of the EphB4 receptor.

55. A compound according to claim 54, wherein the EphB4 is a polypeptide having SEQ ID NOs: 2 or 3.

56. A compound according to claim 54, wherein the EphB4 receptor is a human EphB4 receptor.

57. A compound according to claim 54, wherein the ligand is a polypeptide having SEQ ID NO: 1.

58. A compound according to claim 54, wherein the ligand is a polypeptide selected from the group consisting of polypeptides having SEQ ID NO: 4 through SEQ ID NO: 26.

59. A compound according to claim 54, wherein the ligand is a polypeptide having at least 50% sequence identity to a polypeptide selected from the group consisting of polypeptides having SEQ ID NO: 1 and SEQ ID NO: 4 through SEQ ID NO: 26.

60. A compound according to claim 54, wherein the ligand is a polypeptide having at least 75% sequence identity to a polypeptide selected from the group consisting of polypeptides having SEQ ID NO: 1 and SEQ ID NO: 4 through SEQ ID NO: 26.

61. A compound according to claim 54, wherein the ligand is a polypeptide having at least 90% sequence identity to a polypeptide selected from the group consisting of polypeptides having SEQ ID NO: 1 and SEQ ID NO: 4 through SEQ ID NO: 26.

62. A three-dimensional computer image of the three-dimensional structure of an EphB4-ligand complex, wherein the structure substantially conforms to the three-dimensional coordinates listed in Table 1.

63. A computer-readable medium encoded with a set of three-dimensional coordinates set forth in Table 1, wherein, using a graphical display software program, the three-dimensional coordinates of Table 1 create an electronic file that can be visualized on a computer capable of representing said electronic file as a three-dimensional image.

64. A computer-readable medium encoded with a set of three-dimensional coordinates of a three-dimensional structure which substantially conforms to the three-dimensional coordinates represented in Table 1, wherein, using a graphical display software program, the set of three-dimensional coordinates create an electronic file that can be visualized on a computer capable of representing said electronic file as a three-dimensional image.

65. A method for assaying EphB4 receptor binding to a compound, the method comprising
a) providing an EphB4 receptor bound with a polypeptide having SEQ ID NO: 1;
b) contacting the ligand-bound EphB4 receptor with a compound; and
c) detecting the release of the polypeptide having SEQ ID NO: 1 from the EphB4 receptor, wherein the release of the polypeptide having SEQ ID NO: 1 is indicative of the compound binding to the EphB4 receptor.

66. A method according to claim 65, wherein the EphB4 receptor is a polypeptide having SEQ ID NOs: 2 or 3.

67. A method according to claim 65, wherein the EphB4 receptor consists essentially of EphB4 D-E and J-K loops.

68. A method according to claim 65, wherein the EphB4 receptor consists essentially of Leu-48, Cys-61, Leu-95, Ser-99 Leu-100, Pro-101, Thr-147, Lys-149, Ala-155, and Cys-184 of SEQ ID NO: 27.

69. A method according to claim 65, wherein the EphB4 receptor is a human EphB4 receptor.

70. A method for crystallizing an EphB4 receptor, the method comprising:
a) providing an EphB4 receptor in contact with a first polypeptide having SEQ ID NO: 1; and
b) contacting the EphB4 receptor in contact with the first polypeptide with a second polypeptide having at least 50% sequence identity to SEQ ID NO: 1, but not identical to SEQ ID NO: 1, wherein the EphB4 receptor in contact with the first and second polypeptides forms an EphB4 receptor crystal.

71. A method according to claim 70, wherein the second polypeptide comprises at least 75% sequence identity to SEQ ID NO: 1.

72. A method according to claim 70, wherein the second polypeptide comprises at least 90% sequence identity to SEQ ID NO: 1.

73. A method for crystallizing an EphB4 receptor, the method comprising:
a) providing an EphB4 receptor in contact with a polypeptide having SEQ ID NO: 1; and
b) contacting the EphB4 receptor in contact with the polypeptide with a compound of claim 54,
wherein the EphB4 receptor in contact with the polypeptide and the compound forms an EphB4 receptor crystal.

74. A composition comprising EphB4 receptor, a ligand, and a compound of claim 54.

75. A composition according to claim 74, wherein the EphB4 receptor is a polypeptide having SEQ ID NOs: 2 or 3.

76. A composition according to claim 74, wherein the EphB4 receptor consists essentially of EphB4 D-E and J-K loops.

77. A composition according to claim 74, wherein the EphB4 receptor consists essentially of Leu-48, Cys-61, Leu-95, Ser-99 Leu-100, Pro-101, Thr-147, Lys-149, Ala-155, and Cys-184 of SEQ ID NO: 27.

78. A composition according to claim 74, wherein the EphB4 receptor is a human EphB4 receptor.

79. A composition according to claim 74, wherein the ligand is a polypeptide having SEQ ID NO: 1.

80. A composition according to claim 74, wherein the ligand is a polypeptide selected from the group consisting of polypeptides having SEQ ID NO: 4 through SEQ ID NO: 26.

81. A composition according to claim 74, wherein the ligand is a polypeptide having at least 50% sequence identity to a polypeptide selected from the group consisting of polypeptides having SEQ ID NO: 1 and SEQ ID NO: 4 through SEQ ID NO: 26.

82. A composition according to claim 74, wherein the ligand is a polypeptide having at least 75% sequence identity to a polypeptide selected from the group consisting of polypeptides having SEQ ID NO: 1 and SEQ ID NO: 4 through SEQ ID NO: 26.

83. A composition according to claim 74, wherein the ligand is a polypeptide having at least 90% sequence identity to a polypeptide selected from the group consisting of polypeptides having SEQ ID NO: 1 and SEQ ID NO: 4 through SEQ ID NO: 26.


SEQUENCE LISTING (35 sequences)

SEQ ID NO: 1 - protein, 15 aa, Homo sapiens
TNYLFSPNGPIARAW

SEQ ID NO: 2 - protein, 180 aa, Homo sapiens
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKK

SEQ ID NO: 3 - protein, 182 aa, Homo sapiens
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCA

SEQ ID NO: 4 - protein, 12 aa, Homo sapiens
DALNDWLLFRPW

SEQ ID NO: 5 - protein, 12 aa, Homo sapiens
IPWTQHMAMSPM

SEQ ID NO: 6 - protein, 12 aa, Homo sapiens
SVSVGMKPSPRP

SEQ ID NO: 7 - protein, 12 aa, Homo sapiens
SGHQLLLNKMPN

SEQ ID NO: 8 - protein, 12 aa, Homo sapiens
GPVADAWLVYPR

SEQ ID NO: 9 - protein, 12 aa, Homo sapiens
NPVIGPIQRAWT

SEQ ID NO: 10 - protein, 12 aa, Homo sapiens
DHNHDLYNPWRL

SEQ ID NO: 11 - protein, 12 aa, Homo sapiens
TNYLFSPNGPIA

SEQ ID NO: 12 - protein, 12 aa, Homo sapiens
LPHGPVAAAWDA

SEQ ID NO: 13 - protein, 12 aa, Homo sapiens
TYFDFQAWSIRA

SEQ ID NO: 14 - protein, 12 aa, Homo sapiens
EWYMKFPPEHYF

SEQ ID NO: 15 - protein, 12 aa, Homo sapiens
GPVHRAWEPTSH

SEQ ID NO: 16 - protein, 12 aa, Homo sapiens
SHVGPIMRAWAP

SEQ ID NO: 17 - protein, 12 aa, Homo sapiens
WGIPRAAQVMWT

SEQ ID NO: 18 - protein, 12 aa, Homo sapiens
GPVSKAWQETET

SEQ ID NO: 19 - protein, 12 aa, Homo sapiens
EFFTWRPTYYGI

SEQ ID NO: 20 - protein, 12 aa, Homo sapiens
GPVERAWRPDLI

SEQ ID NO: 21 - protein, 12 aa, Homo sapiens
DHNHNLYNPWRL

SEQ ID NO: 22 - protein, 12 aa, Homo sapiens
FSPQGPAARNFA

SEQ ID NO: 23 - protein, 12 aa, Homo sapiens
SHWPISPYSLLS

SEQ ID NO: 24 - protein, 12 aa, Homo sapiens
WHRYPDPRMLPT

SEQ ID NO: 25 - protein, 12 aa, Homo sapiens
WHWTIEPFAITS

SEQ ID NO: 26 - protein, 12 aa, Homo sapiens
THWCHLLNCAAL

SEQ ID NO: 27 - protein, 987 aa, Homo sapiens <s300> GenBank/NP_004435 2006-12-03 (1)..(987)
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 28 - DNA, 4369 bp, Homo sapiens <s300> GenBank/NM_004444 2006-12-03 (1)..(4369)
ttccagcgcagctcagcccctgcccggcccggcccgcccggctccgcgccgcagtctccctccctcccgctccgtccccgctcgggctcccaccatccccgcccgcgaggagagcactcggcccggcggcgcgagcagagccactccagggagggggggagaccgcgagcggccggctcagcccccgccacccggggcgggaccccgaggccccggagggaccccaactccagccacgtcttgctgcgcgcccgcccggcgcggccactgccagcacgctccgggcccgccgcccgcgcgcgcggcacagacgcggggccacacttggcgccgccgcccggtgccccgcacgctcgcatgggcccgcgctgagggccccgacgaggagtcccgcgcggagtatcggcgtccacccgcccagggagagtcagacctgggggggcgagggccccccaaactcagttcggatcctacccgagtgaggcggcgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggtaaaaaaaaaaaaaaaaaa

SEQ ID NO: 29 - protein, 333 aa, Homo sapiens <s300> NP_004084 2006-11-17 (1)..(333)
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 30 - protein, 14 aa, Homo sapiens
NYLFSPNGPIARAW

SEQ ID NO: 31 - protein, 13 aa, Homo sapiens
YLFSPNGPIARAW

SEQ ID NO: 32 - protein, 12 aa, Homo sapiens
LFSPNGPIARAW

SEQ ID NO: 33 - protein, 12 aa, Homo sapiens
TNYLFSPNGPIA

SEQ ID NO: 34 - protein, 17 aa, Homo sapiens
TNYLFSPNGPIAGSGSK

SEQ ID NO: 35 - protein, 16 aa, Homo sapiens
KFQEFSPNLWGLEFQK